SEQUENCE LISTING (644 sequences)

SEQ ID NO: 1 - protein, 4 aa, Artificial
  Synthetic peptide.  Can be protected or unprotected as shown in specification.
XRFK

SEQ ID NO: 2 - protein, 4 aa, Artificial
  Synthetic peptide.  Can be protected or unprotected as shown in specification.
XREL

SEQ ID NO: 3 - protein, 4 aa, Artificial
  Synthetic peptide.  Can be protected or unprotected as shown in specification.
KFRX

SEQ ID NO: 4 - protein, 4 aa, Artificial
  Synthetic peptide.  Can be protected or unprotected as shown in specification.
LERX

SEQ ID NO: 5 - protein, 4 aa, Artificial
  Synthetic peptide.  Can be protected or unprotected as shown in specification.
XRFK

SEQ ID NO: 6 - protein, 4 aa, Artificial
  Synthetic peptide.  Can be protected or unprotected as shown in specification.
XREL

SEQ ID NO: 7 - protein, 22 aa, Artificial
  Synthetic peptide.  Can be protected or unprotected as shown in specification.
LLEQLNEQFNWVSRLANLTQGE

SEQ ID NO: 8 - protein, 7 aa, Artificial
  Synthetic peptide.  Can be protected or unprotected as shown in specification.
LAEYHAK

SEQ ID NO: 9 - protein, 18 aa, Artificial
  Synthetic peptide.  Can be protected or unprotected as shown in specification.
DWLKAFYDKVAEKLKEAF

SEQ ID NO: 10 - protein, 18 aa, Artificial
  Synthetic peptide.  Can be protected or unprotected as shown in specification.
DWLKAFYDKVAEKLKEAF

SEQ ID NO: 11 - protein, 18 aa, Artificial
  Synthetic peptide.  Can be protected or unprotected as shown in specification.
DWFKAFYDKVAEKLKEAF

SEQ ID NO: 12 - protein, 18 aa, Artificial
  Synthetic peptide.  Can be protected or unprotected as shown in specification.
DWLKAFYDKVAEKFKEAF

SEQ ID NO: 13 - protein, 18 aa, Artificial
  Synthetic peptide.  Can be protected or unprotected as shown in specification.
DWFKAFYDKVAEKFKEAF

SEQ ID NO: 14 - protein, 18 aa, Artificial
  Synthetic peptide.  Can be protected or unprotected as shown in specification.
DWLKAFYDKVFEKFKEFF

SEQ ID NO: 15 - protein, 18 aa, Artificial
  Synthetic peptide.  Can be protected or unprotected as shown in specification.
DWLKAFYDKFFEKFKEFF

SEQ ID NO: 16 - protein, 18 aa, Artificial
  Synthetic peptide.  Can be protected or unprotected as shown in specification.
DWFKAFYDKFFEKFKEFF

SEQ ID NO: 17 - protein, 18 aa, Artificial
  Synthetic peptide.  Can be protected or unprotected as shown in specification.
DWLKAFYDKVAEKLKEFF

SEQ ID NO: 18 - protein, 18 aa, Artificial
  Synthetic peptide.  Can be protected or unprotected as shown in specification.
DWLKAFYDKVFEKFKEAF

SEQ ID NO: 19 - protein, 18 aa, Artificial
  Synthetic peptide.  Can be protected or unprotected as shown in specification.
DWLKAFYDKVFEKLKEFF

SEQ ID NO: 20 - protein, 18 aa, Artificial
  Synthetic peptide.  Can be protected or unprotected as shown in specification.
DWLKAFYDKVAEKFKEFF

SEQ ID NO: 21 - protein, 18 aa, Artificial
  Synthetic peptide.  Can be protected or unprotected as shown in specification.
DWLKAFYDKVFEKFKEFF

SEQ ID NO: 22 - protein, 18 aa, Artificial
  Synthetic peptide.  Can be protected or unprotected as shown in specification.
EWLKLFYEKVLEKFKEAF

SEQ ID NO: 23 - protein, 18 aa, Artificial
  Synthetic peptide.  Can be protected or unprotected as shown in specification.
EWLKAFYDKVAEKFKEAF

SEQ ID NO: 24 - protein, 18 aa, Artificial
  Synthetic peptide.  Can be protected or unprotected as shown in specification.
EWLKAFYDKVAEKLKEFF

SEQ ID NO: 25 - protein, 18 aa, Artificial
  Synthetic peptide.  Can be protected or unprotected as shown in specification.
EWLKAFYDKVFEKFKEAF

SEQ ID NO: 26 - protein, 18 aa, Artificial
  Synthetic peptide.  Can be protected or unprotected as shown in specification.
EWLKAFYDKVFEKLKEFF

SEQ ID NO: 27 - protein, 18 aa, Artificial
  Synthetic peptide.  Can be protected or unprotected as shown in specification.
EWLKAFYDKVAEKFKEFF

SEQ ID NO: 28 - protein, 18 aa, Artificial
  Synthetic peptide.  Can be protected or unprotected as shown in specification.
EWLKAFYDKVFEKFKEFF

SEQ ID NO: 29 - protein, 14 aa, Artificial
  Synthetic peptide.  Can be protected or unprotected as shown in specification.
AFYDKVAEKLKEAF

SEQ ID NO: 30 - protein, 14 aa, Artificial
  Synthetic peptide.  Can be protected or unprotected as shown in specification.
AFYDKVAEKFKEAF

SEQ ID NO: 31 - protein, 14 aa, Artificial
  Synthetic peptide.  Can be protected or unprotected as shown in specification.
AFYDKVAEKFKEAF

SEQ ID NO: 32 - protein, 14 aa, Artificial
  Synthetic peptide.  Can be protected or unprotected as shown in specification.
AFYDKFFEKFKEFF

SEQ ID NO: 33 - protein, 14 aa, Artificial
  Synthetic peptide.  Can be protected or unprotected as shown in specification.
AFYDKFFEKFKEFF

SEQ ID NO: 34 - protein, 14 aa, Artificial
  Synthetic peptide.  Can be protected or unprotected as shown in specification.
AFYDKVAEKFKEAF

SEQ ID NO: 35 - protein, 14 aa, Artificial
  Synthetic peptide.  Can be protected or unprotected as shown in specification.
AFYDKVAEKLKEFF

SEQ ID NO: 36 - protein, 14 aa, Artificial
  Synthetic peptide.  Can be protected or unprotected as shown in specification.
AFYDKVFEKFKEAF

SEQ ID NO: 37 - protein, 14 aa, Artificial
  Synthetic peptide.  Can be protected or unprotected as shown in specification.
AFYDKVFEKLKEFF

SEQ ID NO: 38 - protein, 14 aa, Artificial
  Synthetic peptide.  Can be protected or unprotected as shown in specification.
AFYDKVAEKFKEFF

SEQ ID NO: 39 - protein, 14 aa, Artificial
  Synthetic peptide.  Can be protected or unprotected as shown in specification.
KAFYDKVFEKFKEF

SEQ ID NO: 40 - protein, 14 aa, Artificial
  Synthetic peptide.  Can be protected or unprotected as shown in specification.
LFYEKVLEKFKEAF

SEQ ID NO: 41 - protein, 14 aa, Artificial
  Synthetic peptide.  Can be protected or unprotected as shown in specification.
AFYDKVAEKFKEAF

SEQ ID NO: 42 - protein, 14 aa, Artificial
  Synthetic peptide.  Can be protected or unprotected as shown in specification.
AFYDKVAEKLKEFF

SEQ ID NO: 43 - protein, 14 aa, Artificial
  Synthetic peptide.  Can be protected or unprotected as shown in specification.
AFYDKVFEKFKEAF

SEQ ID NO: 44 - protein, 14 aa, Artificial
  Synthetic peptide.  Can be protected or unprotected as shown in specification.
AFYDKVFEKLKEFF

SEQ ID NO: 45 - protein, 14 aa, Artificial
  Synthetic peptide.  Can be protected or unprotected as shown in specification.
AFYDKVAEKFKEFF

SEQ ID NO: 46 - protein, 14 aa, Artificial
  Synthetic peptide.  Can be protected or unprotected as shown in specification.
AFYDKVFEKFKEFF

SEQ ID NO: 47 - protein, 18 aa, Artificial
  Synthetic peptide.  Can be protected or unprotected as shown in specification.
DWLKALYDKVAEKLKEAL

SEQ ID NO: 48 - protein, 18 aa, Artificial
  Synthetic peptide.  Can be protected or unprotected as shown in specification.
DWFKAFYEKVAEKLKEFF

SEQ ID NO: 49 - protein, 18 aa, Artificial
  Synthetic peptide.  Can be protected or unprotected as shown in specification.
DWFKAFYEKFFEKFKEFF

SEQ ID NO: 50 - protein, 18 aa, Artificial
  Synthetic peptide.  Can be protected or unprotected as shown in specification.
EWLKALYEKVAEKLKEAL

SEQ ID NO: 51 - protein, 18 aa, Artificial
  Synthetic peptide.  Can be protected or unprotected as shown in specification.
EWLKAFYEKVAEKLKEAF

SEQ ID NO: 52 - protein, 18 aa, Artificial
  Synthetic peptide.  Can be protected or unprotected as shown in specification.
EWFKAFYEKVAEKLKEFF

SEQ ID NO: 53 - protein, 18 aa, Artificial
  Synthetic peptide.  Can be protected or unprotected as shown in specification.
EWLKAFYEKVFEKFKEFF

SEQ ID NO: 54 - protein, 18 aa, Artificial
  Synthetic peptide.  Can be protected or unprotected as shown in specification.
EWLKAFYEKFFEKFKEFF

SEQ ID NO: 55 - protein, 18 aa, Artificial
  Synthetic peptide.  Can be protected or unprotected as shown in specification.
EWFKAFYEKFFEKFKEFF

SEQ ID NO: 56 - protein, 18 aa, Artificial
  Synthetic peptide.  Can be protected or unprotected as shown in specification.
DFLKAWYDKVAEKLKEAW

SEQ ID NO: 57 - protein, 18 aa, Artificial
  Synthetic peptide.  Can be protected or unprotected as shown in specification.
EFLKAWYEKVAEKLKEAW

SEQ ID NO: 58 - protein, 18 aa, Artificial
  Synthetic peptide.  Can be protected or unprotected as shown in specification.
DFWKAWYDKVAEKLKEWW

SEQ ID NO: 59 - protein, 18 aa, Artificial
  Synthetic peptide.  Can be protected or unprotected as shown in specification.
EFWKAWYEKVAEKLKEWW

SEQ ID NO: 60 - protein, 18 aa, Artificial
  Synthetic peptide.  Can be protected or unprotected as shown in specification.
DKLKAFYDKVFEWAKEAF

SEQ ID NO: 61 - protein, 18 aa, Artificial
  Synthetic peptide.  Can be protected or unprotected as shown in specification.
DKWKAVYDKFAEAFKEFL

SEQ ID NO: 62 - protein, 18 aa, Artificial
  Synthetic peptide.  Can be protected or unprotected as shown in specification.
EKLKAFYEKVFEWAKEAF

SEQ ID NO: 63 - protein, 18 aa, Artificial
  Synthetic peptide.  Can be protected or unprotected as shown in specification.
EKWKAVYEKFAEAFKEFL

SEQ ID NO: 64 - protein, 18 aa, Artificial
  Synthetic peptide.  Can be protected or unprotected as shown in specification.
DWLKAFVDKFAEKFKEAY

SEQ ID NO: 65 - protein, 18 aa, Artificial
  Synthetic peptide.  Can be protected or unprotected as shown in specification.
EKWKAVYEKFAEAFKEFL

SEQ ID NO: 66 - protein, 18 aa, Artificial
  Synthetic peptide.  Can be protected or unprotected as shown in specification.
DWLKAFVYDKVFKLKEFF

SEQ ID NO: 67 - protein, 18 aa, Artificial
  Synthetic peptide.  Can be protected or unprotected as shown in specification.
EWLKAFVYEKVFKLKEFF

SEQ ID NO: 68 - protein, 18 aa, Artificial
  Synthetic peptide.  Can be protected or unprotected as shown in specification.
DWLRAFYDKVAEKLKEAF

SEQ ID NO: 69 - protein, 18 aa, Artificial
  Synthetic peptide.  Can be protected or unprotected as shown in specification.
EWLRAFYEKVAEKLKEAF

SEQ ID NO: 70 - protein, 18 aa, Artificial
  Synthetic peptide.  Can be protected or unprotected as shown in specification.
DWLKAFYDRVAEKLKEAF

SEQ ID NO: 71 - protein, 18 aa, Artificial
  Synthetic peptide.  Can be protected or unprotected as shown in specification.
EWLKAFYERVAEKLKEAF

SEQ ID NO: 72 - protein, 18 aa, Artificial
  Synthetic peptide.  Can be protected or unprotected as shown in specification.
DWLKAFYDKVAERLKEAF

SEQ ID NO: 73 - protein, 18 aa, Artificial
  Synthetic peptide.  Can be protected or unprotected as shown in specification.
EWLKAFYEKVAERLKEAF

SEQ ID NO: 74 - protein, 18 aa, Artificial
  Synthetic peptide.  Can be protected or unprotected as shown in specification.
DWLKAFYDKVAEKLREAF

SEQ ID NO: 75 - protein, 18 aa, Artificial
  Synthetic peptide.  Can be protected or unprotected as shown in specification.
EWLKAFYEKVAEKLREAF

SEQ ID NO: 76 - protein, 18 aa, Artificial
  Synthetic peptide.  Can be protected or unprotected as shown in specification.
DWLKAFYDRVAERLKEAF

SEQ ID NO: 77 - protein, 18 aa, Artificial
  Synthetic peptide.  Can be protected or unprotected as shown in specification.
EWLKAFYERVAERLKEAF

SEQ ID NO: 78 - protein, 18 aa, Artificial
  Synthetic peptide.  Can be protected or unprotected as shown in specification.
DWLRAFYDKVAEKLREAF

SEQ ID NO: 79 - protein, 18 aa, Artificial
  Synthetic peptide.  Can be protected or unprotected as shown in specification.
EWLRAFYEKVAEKLREAF

SEQ ID NO: 80 - protein, 18 aa, Artificial
  Synthetic peptide.  Can be protected or unprotected as shown in specification.
DWLRAFYDRVAEKLKEAF

SEQ ID NO: 81 - protein, 18 aa, Artificial
  Synthetic peptide.  Can be protected or unprotected as shown in specification.
EWLRAFYERVAEKLKEAF

SEQ ID NO: 82 - protein, 18 aa, Artificial
  Synthetic peptide.  Can be protected or unprotected as shown in specification.
DWLKAFYDKVAERLREAF

SEQ ID NO: 83 - protein, 18 aa, Artificial
  Synthetic peptide.  Can be protected or unprotected as shown in specification.
EWLKAFYEKVAERLREAF

SEQ ID NO: 84 - protein, 18 aa, Artificial
  Synthetic peptide.  Can be protected or unprotected as shown in specification.
DWLRAFYDKVAERLKEAF

SEQ ID NO: 85 - protein, 18 aa, Artificial
  Synthetic peptide.  Can be protected or unprotected as shown in specification.
EWLRAFYEKVAERLKEAF

SEQ ID NO: 86 - protein, 37 aa, Artificial
  Synthetic peptide.  Can be protected or unprotected as shown in specification.
DWLKAFYDKVAEKLKEAFPDWLKAFYDKVAEKLKEAF

SEQ ID NO: 87 - protein, 37 aa, Artificial
  Synthetic peptide.  Can be protected or unprotected as shown in specification.
DWLKAFYDKVAEKLKEFFPDWLKAFYDKVAEKLKEFF

SEQ ID NO: 88 - protein, 37 aa, Artificial
  Synthetic peptide.  Can be protected or unprotected as shown in specification.
DWFKAFYDKVAEKLKEAFPDWFKAFYDKVAEKLKEAF

SEQ ID NO: 89 - protein, 37 aa, Artificial
  Synthetic peptide.  Can be protected or unprotected as shown in specification.
DKLKAFYDKVFEWAKEAFPDKLKAFYDKVFEWLKEAF

SEQ ID NO: 90 - protein, 37 aa, Artificial
  Synthetic peptide.  Can be protected or unprotected as shown in specification.
DKWKAVYDKFAEAFKEFLPDKWKAVYDKFAEAFKEFL

SEQ ID NO: 91 - protein, 37 aa, Artificial
  Synthetic peptide.  Can be protected or unprotected as shown in specification.
DWFKAFYDKVAEKFKEAFPDWFKAFYDKVAEKFKEAF

SEQ ID NO: 92 - protein, 37 aa, Artificial
  Synthetic peptide.  Can be protected or unprotected as shown in specification.
DWLKAFVYDKVFKLKEFFPDWLKAFVYDKVFKLKEFF

SEQ ID NO: 93 - protein, 37 aa, Artificial
  Synthetic peptide.  Can be protected or unprotected as shown in specification.
DWLKAFYDKFAEKFKEFFPDWLKAFYDKFAEKFKEFF

SEQ ID NO: 94 - protein, 18 aa, Artificial
  Synthetic peptide.  Can be protected or unprotected as shown in specification.
EWFKAFYEKVAEKFKEAF

SEQ ID NO: 95 - protein, 14 aa, Artificial
  Synthetic peptide.  Can be protected or unprotected as shown in specification.
DWFKAFYDKVAEKF

SEQ ID NO: 96 - protein, 14 aa, Artificial
  Synthetic peptide.  Can be protected or unprotected as shown in specification.
FKAFYDKVAEKFKE

SEQ ID NO: 97 - protein, 14 aa, Artificial
  Synthetic peptide.  Can be protected or unprotected as shown in specification.
FKAFYEKVAEKFKE

SEQ ID NO: 98 - protein, 14 aa, Artificial
  Synthetic peptide.  Can be protected or unprotected as shown in specification.
FKAFYDKVAEKFKE

SEQ ID NO: 99 - protein, 14 aa, Artificial
  Synthetic peptide.  Can be protected or unprotected as shown in specification.
FKAFYEKVAEKFKE

SEQ ID NO: 100 - protein, 18 aa, Artificial
  Synthetic peptide.  Can be protected or unprotected as shown in specification.
DWFKAFYDKVAEKFKEAF

SEQ ID NO: 101 - protein, 18 aa, Artificial
  Synthetic peptide.  Can be protected or unprotected as shown in specification.
EWFKAFYEKVAEKFKEAF

SEQ ID NO: 102 - protein, 14 aa, Artificial
  Synthetic peptide.  Can be protected or unprotected as shown in specification.
AFYDKVAEKFKEAF

SEQ ID NO: 103 - protein, 14 aa, Artificial
  Synthetic peptide.  Can be protected or unprotected as shown in specification.
DWFKAFYDKVAEKF

SEQ ID NO: 104 - protein, 18 aa, Artificial
  Synthetic peptide.  Can be protected or unprotected as shown in specification.
DWLKAFYDKVFEKFKEFF

SEQ ID NO: 105 - protein, 18 aa, Artificial
  Synthetic peptide.  Can be protected or unprotected as shown in specification.
EWLKAFYEKVFEKFKEFF

SEQ ID NO: 106 - protein, 14 aa, Artificial
  Synthetic peptide.  Can be protected or unprotected as shown in specification.
AFYDKVFEKFKEFF

SEQ ID NO: 107 - protein, 14 aa, Artificial
  Synthetic peptide.  Can be protected or unprotected as shown in specification.
AFYEKVFEKFKEFF

SEQ ID NO: 108 - protein, 14 aa, Artificial
  Synthetic peptide.  Can be protected or unprotected as shown in specification.
DWLKAFYDKVFEKF

SEQ ID NO: 109 - protein, 14 aa, Artificial
  Synthetic peptide.  Can be protected or unprotected as shown in specification.
EWLKAFYEKVFEKF

SEQ ID NO: 110 - protein, 14 aa, Artificial
  Synthetic peptide.  Can be protected or unprotected as shown in specification.
LKAFYDKVFEKFKE

SEQ ID NO: 111 - protein, 14 aa, Artificial
  Synthetic peptide.  Can be protected or unprotected as shown in specification.
LKAFYEKVFEKFKE

SEQ ID NO: 112 - protein, 18 aa, Artificial
  Synthetic peptide.  Can be protected or unprotected as shown in specification.
DKWKAVYDKFAEAFKEFL

SEQ ID NO: 113 - protein, 18 aa, Artificial
  Synthetic peptide.  Can be protected or unprotected as shown in specification.
DKLKAFYDKVFEWAKEAF

SEQ ID NO: 114 - protein, 18 aa, Artificial
  Synthetic peptide.  Can be protected or unprotected as shown in specification.
DVWKAAYDKFAEKFKEFF

SEQ ID NO: 115 - protein, 18 aa, Artificial
  Synthetic peptide.  Can be protected or unprotected as shown in specification.
DVWKAFYDKFAEKFKEAF

SEQ ID NO: 116 - protein, 18 aa, Artificial
  Synthetic peptide.  Can be protected or unprotected as shown in specification.
DFWKAFYDKVAEKFKEAF

SEQ ID NO: 117 - protein, 18 aa, Artificial
  Synthetic peptide.  Can be protected or unprotected as shown in specification.
FFEKFKEAFKDYAAKWVD

SEQ ID NO: 118 - protein, 18 aa, Artificial
  Synthetic peptide.  Can be protected or unprotected as shown in specification.
FAEKFKEAFKDYFAKWVD

SEQ ID NO: 119 - protein, 18 aa, Artificial
  Synthetic peptide.  Can be protected or unprotected as shown in specification.
FAEKFKEAVKDYFAKWFD

SEQ ID NO: 120 - protein, 3 aa, Artificial
  Synthetic peptide.  Can be protected or unprotected as shown in specification.
KRS

SEQ ID NO: 121 - protein, 3 aa, Artificial
  Synthetic peptide.  Can be protected or unprotected as shown in specification.
KRT

SEQ ID NO: 122 - protein, 3 aa, Artificial
  Synthetic peptide.  Can be protected or unprotected as shown in specification.
WRI

SEQ ID NO: 123 - protein, 3 aa, Artificial
  Synthetic peptide.  Can be protected or unprotected as shown in specification.
WRL

SEQ ID NO: 124 - protein, 3 aa, Artificial
  Synthetic peptide.  Can be protected or unprotected as shown in specification.
FRI

SEQ ID NO: 125 - protein, 3 aa, Artificial
  Synthetic peptide.  Can be protected or unprotected as shown in specification.
FRL

SEQ ID NO: 126 - protein, 3 aa, Artificial
  Synthetic peptide.  Can be protected or unprotected as shown in specification.
KES

SEQ ID NO: 127 - protein, 3 aa, Artificial
  Synthetic peptide.  Can be protected or unprotected as shown in specification.
KET

SEQ ID NO: 128 - protein, 3 aa, Artificial
  Synthetic peptide.  Can be protected or unprotected as shown in specification.
KDS

SEQ ID NO: 129 - protein, 3 aa, Artificial
  Synthetic peptide.  Can be protected or unprotected as shown in specification.
KDT

SEQ ID NO: 130 - protein, 3 aa, Artificial
  Synthetic peptide.  Can be protected or unprotected as shown in specification.
KRS

SEQ ID NO: 131 - protein, 3 aa, Artificial
  Synthetic peptide.  Can be protected or unprotected as shown in specification.
KRT

SEQ ID NO: 132 - protein, 3 aa, Artificial
  Synthetic peptide.  Can be protected or unprotected as shown in specification.
LES

SEQ ID NO: 133 - protein, 3 aa, Artificial
  Synthetic peptide.  Can be protected or unprotected as shown in specification.
LET

SEQ ID NO: 134 - protein, 3 aa, Artificial
  Synthetic peptide.  Can be protected or unprotected as shown in specification.
WRS

SEQ ID NO: 135 - protein, 3 aa, Artificial
  Synthetic peptide.  Can be protected or unprotected as shown in specification.
WDS

SEQ ID NO: 136 - protein, 3 aa, Artificial
  Synthetic peptide.  Can be protected or unprotected as shown in specification.
WES

SEQ ID NO: 137 - protein, 3 aa, Artificial
  Synthetic peptide.  Can be protected or unprotected as shown in specification.
WRS

SEQ ID NO: 138 - protein, 3 aa, Artificial
  Synthetic peptide.  Can be protected or unprotected as shown in specification.
KEL

SEQ ID NO: 139 - protein, 3 aa, Artificial
  Synthetic peptide.  Can be protected or unprotected as shown in specification.
LRS

SEQ ID NO: 140 - protein, 3 aa, Artificial
  Synthetic peptide.  Can be protected or unprotected as shown in specification.
LDS

SEQ ID NO: 141 - protein, 3 aa, Artificial
  Synthetic peptide.  Can be protected or unprotected as shown in specification.
LES

SEQ ID NO: 142 - protein, 3 aa, Artificial
  Synthetic peptide.  Can be protected or unprotected as shown in specification.
LRS

SEQ ID NO: 143 - protein, 3 aa, Artificial
  Synthetic peptide.  Can be protected or unprotected as shown in specification.
LRT

SEQ ID NO: 144 - protein, 3 aa, Artificial
  Synthetic peptide.  Can be protected or unprotected as shown in specification.
EDY

SEQ ID NO: 145 - protein, 3 aa, Artificial
  Synthetic peptide.  Can be protected or unprotected as shown in specification.
KRS

SEQ ID NO: 146 - protein, 3 aa, Artificial
  Synthetic peptide.  Can be protected or unprotected as shown in specification.
WRI

SEQ ID NO: 147 - protein, 3 aa, Artificial
  Synthetic peptide.  Can be protected or unprotected as shown in specification.
WRL

SEQ ID NO: 148 - protein, 3 aa, Artificial
  Synthetic peptide.  Can be protected or unprotected as shown in specification.
FRI

SEQ ID NO: 149 - protein, 3 aa, Artificial
  Synthetic peptide.  Can be protected or unprotected as shown in specification.
FRL

SEQ ID NO: 150 - protein, 3 aa, Artificial
  Synthetic peptide.  Can be protected or unprotected as shown in specification.
WRF

SEQ ID NO: 151 - protein, 3 aa, Artificial
  Synthetic peptide.  Can be protected or unprotected as shown in specification.
WRY

SEQ ID NO: 152 - protein, 3 aa, Artificial
  Synthetic peptide.  Can be protected or unprotected as shown in specification.
WRF

SEQ ID NO: 153 - protein, 3 aa, Artificial
  Synthetic peptide.  Can be protected or unprotected as shown in specification.
WRY

SEQ ID NO: 154 - protein, 3 aa, Artificial
  Synthetic peptide.  Can be protected or unprotected as shown in specification.
XRS

SEQ ID NO: 155 - protein, 3 aa, Artificial
  Synthetic peptide.  Can be protected or unprotected as shown in specification.
KRS

SEQ ID NO: 156 - protein, 3 aa, Artificial
  Synthetic peptide.  Can be protected or unprotected as shown in specification.
KRT

SEQ ID NO: 157 - protein, 3 aa, Artificial
  Synthetic peptide.  Can be protected or unprotected as shown in specification.
LDT

SEQ ID NO: 158 - protein, 3 aa, Artificial
  Synthetic peptide.  Can be protected or unprotected as shown in specification.
LET

SEQ ID NO: 159 - protein, 3 aa, Artificial
  Synthetic peptide.  Can be protected or unprotected as shown in specification.
LRT

SEQ ID NO: 160 - protein, 3 aa, Artificial
  Synthetic peptide.  Can be protected or unprotected as shown in specification.
XRS

SEQ ID NO: 161 - protein, 3 aa, Artificial
  Synthetic peptide.  Can be protected or unprotected as shown in specification.
XDS

SEQ ID NO: 162 - protein, 3 aa, Artificial
  Synthetic peptide.  Can be protected or unprotected as shown in specification.
XES

SEQ ID NO: 163 - protein, 3 aa, Artificial
  Synthetic peptide.  Can be protected or unprotected as shown in specification.
KRS

SEQ ID NO: 164 - protein, 3 aa, Artificial
  Synthetic peptide.  Can be protected or unprotected as shown in specification.
KRT

SEQ ID NO: 165 - protein, 3 aa, Artificial
  Synthetic peptide.  Can be protected or unprotected as shown in specification.
KES

SEQ ID NO: 166 - protein, 3 aa, Artificial
  Synthetic peptide.  Can be protected or unprotected as shown in specification.
KET

SEQ ID NO: 167 - protein, 3 aa, Artificial
  Synthetic peptide.  Can be protected or unprotected as shown in specification.
KDS

SEQ ID NO: 168 - protein, 3 aa, Artificial
  Synthetic peptide.  Can be protected or unprotected as shown in specification.
KDT

SEQ ID NO: 169 - protein, 3 aa, Artificial
  Synthetic peptide.  Can be protected or unprotected as shown in specification.
KEL

SEQ ID NO: 170 - protein, 3 aa, Artificial
  Synthetic peptide.  Can be protected or unprotected as shown in specification.
KRL

SEQ ID NO: 171 - protein, 3 aa, Artificial
  Synthetic peptide.  Can be protected or unprotected as shown in specification.
KRT

SEQ ID NO: 172 - protein, 3 aa, Artificial
  Synthetic peptide.  Can be protected or unprotected as shown in specification.
KES

SEQ ID NO: 173 - protein, 3 aa, Artificial
  Synthetic peptide.  Can be protected or unprotected as shown in specification.
KET

SEQ ID NO: 174 - protein, 3 aa, Artificial
  Synthetic peptide.  Can be protected or unprotected as shown in specification.
KDS

SEQ ID NO: 175 - protein, 3 aa, Artificial
  Synthetic peptide.  Can be protected or unprotected as shown in specification.
KDT

SEQ ID NO: 176 - protein, 3 aa, Artificial
  Synthetic peptide.  Can be protected or unprotected as shown in specification.
KRS

SEQ ID NO: 177 - protein, 3 aa, Artificial
  Synthetic peptide.  Can be protected or unprotected as shown in specification.
KEL

SEQ ID NO: 178 - protein, 3 aa, Artificial
  Synthetic peptide.  Can be protected or unprotected as shown in specification.
KDS

SEQ ID NO: 179 - protein, 3 aa, Artificial
  Synthetic peptide.  Can be protected or unprotected as shown in specification.
KDT

SEQ ID NO: 180 - protein, 3 aa, Artificial
  Synthetic peptide.  Can be protected or unprotected as shown in specification.
KRT

SEQ ID NO: 181 - protein, 3 aa, Artificial
  Synthetic peptide.  Can be protected or unprotected as shown in specification.
KEL

SEQ ID NO: 182 - protein, 3 aa, Artificial
  Synthetic peptide.  Can be protected or unprotected as shown in specification.
XES

SEQ ID NO: 183 - protein, 3 aa, Artificial
  Synthetic peptide.  Can be protected or unprotected as shown in specification.
XDS

SEQ ID NO: 184 - protein, 3 aa, Artificial
  Synthetic peptide.  Can be protected or unprotected as shown in specification.
XDT

SEQ ID NO: 185 - protein, 3 aa, Artificial
  Synthetic peptide.  Can be protected or unprotected as shown in specification.
XRT

SEQ ID NO: 186 - protein, 3 aa, Artificial
  Synthetic peptide.  Can be protected or unprotected as shown in specification.
XET

SEQ ID NO: 187 - protein, 3 aa, Artificial
  Synthetic peptide.  Can be protected or unprotected as shown in specification.
WDI

SEQ ID NO: 188 - protein, 3 aa, Artificial
  Synthetic peptide.  Can be protected or unprotected as shown in specification.
WRI

SEQ ID NO: 189 - protein, 3 aa, Artificial
  Synthetic peptide.  Can be protected or unprotected as shown in specification.
WEI

SEQ ID NO: 190 - protein, 3 aa, Artificial
  Synthetic peptide.  Can be protected or unprotected as shown in specification.
WDL

SEQ ID NO: 191 - protein, 3 aa, Artificial
  Synthetic peptide.  Can be protected or unprotected as shown in specification.
WEL

SEQ ID NO: 192 - protein, 3 aa, Artificial
  Synthetic peptide.  Can be protected or unprotected as shown in specification.
FDI

SEQ ID NO: 193 - protein, 3 aa, Artificial
  Synthetic peptide.  Can be protected or unprotected as shown in specification.
FDL

SEQ ID NO: 194 - protein, 3 aa, Artificial
  Synthetic peptide.  Can be protected or unprotected as shown in specification.
FEL

SEQ ID NO: 195 - protein, 3 aa, Artificial
  Synthetic peptide.  Can be protected or unprotected as shown in specification.
WRF

SEQ ID NO: 196 - protein, 3 aa, Artificial
  Synthetic peptide.  Can be protected or unprotected as shown in specification.
WEF

SEQ ID NO: 197 - protein, 3 aa, Artificial
  Synthetic peptide.  Can be protected or unprotected as shown in specification.
WDF

SEQ ID NO: 198 - protein, 3 aa, Artificial
  Synthetic peptide.  Can be protected or unprotected as shown in specification.
WDY

SEQ ID NO: 199 - protein, 3 aa, Artificial
  Synthetic peptide.  Can be protected or unprotected as shown in specification.
WRY

SEQ ID NO: 200 - protein, 3 aa, Artificial
  Synthetic peptide.  Can be protected or unprotected as shown in specification.
WEY

SEQ ID NO: 201 - protein, 3 aa, Artificial
  Synthetic peptide.  Can be protected or unprotected as shown in specification.
WRT

SEQ ID NO: 202 - protein, 3 aa, Artificial
  Synthetic peptide.  Can be protected or unprotected as shown in specification.
WDT

SEQ ID NO: 203 - protein, 3 aa, Artificial
  Synthetic peptide.  Can be protected or unprotected as shown in specification.
WET

SEQ ID NO: 204 - protein, 3 aa, Artificial
  Synthetic peptide.  Can be protected or unprotected as shown in specification.
FRX

SEQ ID NO: 205 - protein, 3 aa, Artificial
  Synthetic peptide.  Can be protected or unprotected as shown in specification.
FEX

SEQ ID NO: 206 - protein, 3 aa, Artificial
  Synthetic peptide.  Can be protected or unprotected as shown in specification.
FDX

SEQ ID NO: 207 - protein, 3 aa, Artificial
  Synthetic peptide.  Can be protected or unprotected as shown in specification.
EHY

SEQ ID NO: 208 - protein, 3 aa, Artificial
  Synthetic peptide.  Can be protected or unprotected as shown in specification.
LHS

SEQ ID NO: 209 - protein, 3 aa, Artificial
  Synthetic peptide.  Can be protected or unprotected as shown in specification.
LHT

SEQ ID NO: 210 - protein, 3 aa, Artificial
  Synthetic peptide.  Can be protected or unprotected as shown in specification.
KHS

SEQ ID NO: 211 - protein, 3 aa, Artificial
  Synthetic peptide.  Can be protected or unprotected as shown in specification.
KHT

SEQ ID NO: 212 - protein, 3 aa, Artificial
  Synthetic peptide.  Can be protected or unprotected as shown in specification.
KHL

SEQ ID NO: 213 - protein, 3 aa, Artificial
  Synthetic peptide.  Can be protected or unprotected as shown in specification.
KHS

SEQ ID NO: 214 - protein, 3 aa, Artificial
  Synthetic peptide.  Can be protected or unprotected as shown in specification.
KHT

SEQ ID NO: 215 - protein, 3 aa, Artificial
  Synthetic peptide.  Can be protected or unprotected as shown in specification.
KHL

SEQ ID NO: 216 - protein, 3 aa, Artificial
  Synthetic peptide.  Can be protected or unprotected as shown in specification.
XHS

SEQ ID NO: 217 - protein, 3 aa, Artificial
  Synthetic peptide.  Can be protected or unprotected as shown in specification.
XHT

SEQ ID NO: 218 - protein, 3 aa, Artificial
  Synthetic peptide.  Can be protected or unprotected as shown in specification.
FHI

SEQ ID NO: 219 - protein, 3 aa, Artificial
  Synthetic peptide.  Can be protected or unprotected as shown in specification.
FHL

SEQ ID NO: 220 - protein, 3 aa, Artificial
  Synthetic peptide.  Can be protected or unprotected as shown in specification.
FHX

SEQ ID NO: 221 - protein, 3 aa, Artificial
  Synthetic peptide.  Can be protected or unprotected as shown in specification.
FKL

SEQ ID NO: 222 - protein, 3 aa, Artificial
  Synthetic peptide.  Can be protected or unprotected as shown in specification.
WHI

SEQ ID NO: 223 - protein, 3 aa, Artificial
  Synthetic peptide.  Can be protected or unprotected as shown in specification.
WHL

SEQ ID NO: 224 - protein, 3 aa, Artificial
  Synthetic peptide.  Can be protected or unprotected as shown in specification.
WHF

SEQ ID NO: 225 - protein, 3 aa, Artificial
  Synthetic peptide.  Can be protected or unprotected as shown in specification.
WHY

SEQ ID NO: 226 - protein, 3 aa, Artificial
  Synthetic peptide.  Can be protected or unprotected as shown in specification.
FKL

SEQ ID NO: 227 - protein, 3 aa, Artificial
  Synthetic peptide.  Can be protected or unprotected as shown in specification.
KHS

SEQ ID NO: 228 - protein, 3 aa, Artificial
  Synthetic peptide.  Can be protected or unprotected as shown in specification.
KHT

SEQ ID NO: 229 - protein, 3 aa, Artificial
  Synthetic peptide.  Can be protected or unprotected as shown in specification.
KHL

SEQ ID NO: 230 - protein, 3 aa, Artificial
  Synthetic peptide.  Can be protected or unprotected as shown in specification.
LHS

SEQ ID NO: 231 - protein, 3 aa, Artificial
  Synthetic peptide.  Can be protected or unprotected as shown in specification.
LHT

SEQ ID NO: 232 - protein, 3 aa, Artificial
  Synthetic peptide.  Can be protected or unprotected as shown in specification.
KHS

SEQ ID NO: 233 - protein, 3 aa, Artificial
  Synthetic peptide.  Can be protected or unprotected as shown in specification.
KHT

SEQ ID NO: 234 - protein, 3 aa, Artificial
  Synthetic peptide.  Can be protected or unprotected as shown in specification.
KHL

SEQ ID NO: 235 - protein, 3 aa, Artificial
  Synthetic peptide.  Can be protected or unprotected as shown in specification.
KHS

SEQ ID NO: 236 - protein, 3 aa, Artificial
  Synthetic peptide.  Can be protected or unprotected as shown in specification.
KHT

SEQ ID NO: 237 - protein, 3 aa, Artificial
  Synthetic peptide.  Can be protected or unprotected as shown in specification.
XHS

SEQ ID NO: 238 - protein, 3 aa, Artificial
  Synthetic peptide.  Can be protected or unprotected as shown in specification.
FHI

SEQ ID NO: 239 - protein, 3 aa, Artificial
  Synthetic peptide.  Can be protected or unprotected as shown in specification.
FHL

SEQ ID NO: 240 - protein, 3 aa, Artificial
  Synthetic peptide.  Can be protected or unprotected as shown in specification.
FHX

SEQ ID NO: 241 - protein, 3 aa, Artificial
  Synthetic peptide.  Can be protected or unprotected as shown in specification.
WHS

SEQ ID NO: 242 - protein, 3 aa, Artificial
  Synthetic peptide.  Can be protected or unprotected as shown in specification.
WHI

SEQ ID NO: 243 - protein, 3 aa, Artificial
  Synthetic peptide.  Can be protected or unprotected as shown in specification.
WHL

SEQ ID NO: 244 - protein, 3 aa, Artificial
  Synthetic peptide.  Can be protected or unprotected as shown in specification.
WHF

SEQ ID NO: 245 - protein, 3 aa, Artificial
  Synthetic peptide.  Can be protected or unprotected as shown in specification.
WHY

SEQ ID NO: 246 - protein, 3 aa, Artificial
  Synthetic peptide.  Can be protected or unprotected as shown in specification.
WHT

SEQ ID NO: 247 - protein, 3 aa, Artificial
  Synthetic peptide.  Can be protected or unprotected as shown in specification.
KHS

SEQ ID NO: 248 - protein, 3 aa, Artificial
  Synthetic peptide.  Can be protected or unprotected as shown in specification.
KHT

SEQ ID NO: 249 - protein, 4 aa, Artificial
  Synthetic peptide.  Can be protected or unprotected as shown in specification.
KRDS

SEQ ID NO: 250 - protein, 4 aa, Artificial
  Synthetic peptide.  Can be protected or unprotected as shown in specification.
KRDT

SEQ ID NO: 251 - protein, 4 aa, Artificial
  Synthetic peptide.  Can be protected or unprotected as shown in specification.
WRDI

SEQ ID NO: 252 - protein, 4 aa, Artificial
  Synthetic peptide.  Can be protected or unprotected as shown in specification.
WRDL

SEQ ID NO: 253 - protein, 4 aa, Artificial
  Synthetic peptide.  Can be protected or unprotected as shown in specification.
FRDL

SEQ ID NO: 254 - protein, 4 aa, Artificial
  Synthetic peptide.  Can be protected or unprotected as shown in specification.
FRDI

SEQ ID NO: 255 - protein, 4 aa, Artificial
  Synthetic peptide.  Can be protected or unprotected as shown in specification.
FRDX

SEQ ID NO: 256 - protein, 4 aa, Artificial
  Synthetic peptide.  Can be protected or unprotected as shown in specification.
FREX

SEQ ID NO: 257 - protein, 4 aa, Artificial
  Synthetic peptide.  Can be protected or unprotected as shown in specification.
FREI

SEQ ID NO: 258 - protein, 4 aa, Artificial
  Synthetic peptide.  Can be protected or unprotected as shown in specification.
FDRI

SEQ ID NO: 259 - protein, 4 aa, Artificial
  Synthetic peptide.  Can be protected or unprotected as shown in specification.
FERI

SEQ ID NO: 260 - protein, 4 aa, Artificial
  Synthetic peptide.  Can be protected or unprotected as shown in specification.
FDRL

SEQ ID NO: 261 - protein, 4 aa, Artificial
  Synthetic peptide.  Can be protected or unprotected as shown in specification.
FREL

SEQ ID NO: 262 - protein, 4 aa, Artificial
  Synthetic peptide.  Can be protected or unprotected as shown in specification.
FERL

SEQ ID NO: 263 - protein, 4 aa, Artificial
  Synthetic peptide.  Can be protected or unprotected as shown in specification.
FDRX

SEQ ID NO: 264 - protein, 4 aa, Artificial
  Synthetic peptide.  Can be protected or unprotected as shown in specification.
FERX

SEQ ID NO: 265 - protein, 4 aa, Artificial
  Synthetic peptide.  Can be protected or unprotected as shown in specification.
KERS

SEQ ID NO: 266 - protein, 4 aa, Artificial
  Synthetic peptide.  Can be protected or unprotected as shown in specification.
KERT

SEQ ID NO: 267 - protein, 4 aa, Artificial
  Synthetic peptide.  Can be protected or unprotected as shown in specification.
KDRS

SEQ ID NO: 268 - protein, 4 aa, Artificial
  Synthetic peptide.  Can be protected or unprotected as shown in specification.
KDRT

SEQ ID NO: 269 - protein, 4 aa, Artificial
  Synthetic peptide.  Can be protected or unprotected as shown in specification.
KRES

SEQ ID NO: 270 - protein, 4 aa, Artificial
  Synthetic peptide.  Can be protected or unprotected as shown in specification.
KRET

SEQ ID NO: 271 - protein, 4 aa, Artificial
  Synthetic peptide.  Can be protected or unprotected as shown in specification.
LERS

SEQ ID NO: 272 - protein, 4 aa, Artificial
  Synthetic peptide.  Can be protected or unprotected as shown in specification.
LERT

SEQ ID NO: 273 - protein, 4 aa, Artificial
  Synthetic peptide.  Can be protected or unprotected as shown in specification.
WRDS

SEQ ID NO: 274 - protein, 4 aa, Artificial
  Synthetic peptide.  Can be protected or unprotected as shown in specification.
WDRS

SEQ ID NO: 275 - protein, 4 aa, Artificial
  Synthetic peptide.  Can be protected or unprotected as shown in specification.
WERS

SEQ ID NO: 276 - protein, 4 aa, Artificial
  Synthetic peptide.  Can be protected or unprotected as shown in specification.
WRES

SEQ ID NO: 277 - protein, 4 aa, Artificial
  Synthetic peptide.  Can be protected or unprotected as shown in specification.
KERL

SEQ ID NO: 278 - protein, 4 aa, Artificial
  Synthetic peptide.  Can be protected or unprotected as shown in specification.
LRDS

SEQ ID NO: 279 - protein, 4 aa, Artificial
  Synthetic peptide.  Can be protected or unprotected as shown in specification.
LDRS

SEQ ID NO: 280 - protein, 4 aa, Artificial
  Synthetic peptide.  Can be protected or unprotected as shown in specification.
LERS

SEQ ID NO: 281 - protein, 4 aa, Artificial
  Synthetic peptide.  Can be protected or unprotected as shown in specification.
LRES

SEQ ID NO: 282 - protein, 4 aa, Artificial
  Synthetic peptide.  Can be protected or unprotected as shown in specification.
LRDT

SEQ ID NO: 283 - protein, 4 aa, Artificial
  Synthetic peptide.  Can be protected or unprotected as shown in specification.
EDRY

SEQ ID NO: 284 - protein, 4 aa, Artificial
  Synthetic peptide.  Can be protected or unprotected as shown in specification.
KRDS

SEQ ID NO: 285 - protein, 4 aa, Artificial
  Synthetic peptide.  Can be protected or unprotected as shown in specification.
WRDI

SEQ ID NO: 286 - protein, 4 aa, Artificial
  Synthetic peptide.  Can be protected or unprotected as shown in specification.
WRDL

SEQ ID NO: 287 - protein, 4 aa, Artificial
  Synthetic peptide.  Can be protected or unprotected as shown in specification.
FRDI

SEQ ID NO: 288 - protein, 4 aa, Artificial
  Synthetic peptide.  Can be protected or unprotected as shown in specification.
FRDL

SEQ ID NO: 289 - protein, 4 aa, Artificial
  Synthetic peptide.  Can be protected or unprotected as shown in specification.
WRDF

SEQ ID NO: 290 - protein, 4 aa, Artificial
  Synthetic peptide.  Can be protected or unprotected as shown in specification.
WRDY

SEQ ID NO: 291 - protein, 4 aa, Artificial
  Synthetic peptide.  Can be protected or unprotected as shown in specification.
WRDF

SEQ ID NO: 292 - protein, 4 aa, Artificial
  Synthetic peptide.  Can be protected or unprotected as shown in specification.
WRDY

SEQ ID NO: 293 - protein, 4 aa, Artificial
  Synthetic peptide.  Can be protected or unprotected as shown in specification.
XRES

SEQ ID NO: 294 - protein, 4 aa, Artificial
  Synthetic peptide.  Can be protected or unprotected as shown in specification.
KRDS

SEQ ID NO: 295 - protein, 4 aa, Artificial
  Synthetic peptide.  Can be protected or unprotected as shown in specification.
KRDT

SEQ ID NO: 296 - protein, 4 aa, Artificial
  Synthetic peptide.  Can be protected or unprotected as shown in specification.
LDRT

SEQ ID NO: 297 - protein, 4 aa, Artificial
  Synthetic peptide.  Can be protected or unprotected as shown in specification.
LERT

SEQ ID NO: 298 - protein, 4 aa, Artificial
  Synthetic peptide.  Can be protected or unprotected as shown in specification.
LRET

SEQ ID NO: 299 - protein, 4 aa, Artificial
  Synthetic peptide.  Can be protected or unprotected as shown in specification.
XRDS

SEQ ID NO: 300 - protein, 4 aa, Artificial
  Synthetic peptide.  Can be protected or unprotected as shown in specification.
XDRS

SEQ ID NO: 301 - protein, 4 aa, Artificial
  Synthetic peptide.  Can be protected or unprotected as shown in specification.
XERS

SEQ ID NO: 302 - protein, 4 aa, Artificial
  Synthetic peptide.  Can be protected or unprotected as shown in specification.
XRES

SEQ ID NO: 303 - protein, 4 aa, Artificial
  Synthetic peptide.  Can be protected or unprotected as shown in specification.
KRDS

SEQ ID NO: 304 - protein, 4 aa, Artificial
  Synthetic peptide.  Can be protected or unprotected as shown in specification.
KRDT

SEQ ID NO: 305 - protein, 4 aa, Artificial
  Synthetic peptide.  Can be protected or unprotected as shown in specification.
KERS

SEQ ID NO: 306 - protein, 4 aa, Artificial
  Synthetic peptide.  Can be protected or unprotected as shown in specification.
KERT

SEQ ID NO: 307 - protein, 4 aa, Artificial
  Synthetic peptide.  Can be protected or unprotected as shown in specification.
KDRS

SEQ ID NO: 308 - protein, 4 aa, Artificial
  Synthetic peptide.  Can be protected or unprotected as shown in specification.
KDRT

SEQ ID NO: 309 - protein, 4 aa, Artificial
  Synthetic peptide.  Can be protected or unprotected as shown in specification.
KRES

SEQ ID NO: 310 - protein, 4 aa, Artificial
  Synthetic peptide.  Can be protected or unprotected as shown in specification.
KRET

SEQ ID NO: 311 - protein, 4 aa, Artificial
  Synthetic peptide.  Can be protected or unprotected as shown in specification.
KERL

SEQ ID NO: 312 - protein, 4 aa, Artificial
  Synthetic peptide.  Can be protected or unprotected as shown in specification.
KREL

SEQ ID NO: 313 - protein, 4 aa, Artificial
  Synthetic peptide.  Can be protected or unprotected as shown in specification.
KRDT

SEQ ID NO: 314 - protein, 4 aa, Artificial
  Synthetic peptide.  Can be protected or unprotected as shown in specification.
KERS

SEQ ID NO: 315 - protein, 4 aa, Artificial
  Synthetic peptide.  Can be protected or unprotected as shown in specification.
KERT

SEQ ID NO: 316 - protein, 4 aa, Artificial
  Synthetic peptide.  Can be protected or unprotected as shown in specification.
KDRS

SEQ ID NO: 317 - protein, 4 aa, Artificial
  Synthetic peptide.  Can be protected or unprotected as shown in specification.
KDRT

SEQ ID NO: 318 - protein, 4 aa, Artificial
  Synthetic peptide.  Can be protected or unprotected as shown in specification.
KRES

SEQ ID NO: 319 - protein, 4 aa, Artificial
  Synthetic peptide.  Can be protected or unprotected as shown in specification.
KRET

SEQ ID NO: 320 - protein, 4 aa, Artificial
  Synthetic peptide.  Can be protected or unprotected as shown in specification.
KERL

SEQ ID NO: 321 - protein, 4 aa, Artificial
  Synthetic peptide.  Can be protected or unprotected as shown in specification.
KRDS

SEQ ID NO: 322 - protein, 4 aa, Artificial
  Synthetic peptide.  Can be protected or unprotected as shown in specification.
KRDT

SEQ ID NO: 323 - protein, 4 aa, Artificial
  Synthetic peptide.  Can be protected or unprotected as shown in specification.
KERS

SEQ ID NO: 324 - protein, 4 aa, Artificial
  Synthetic peptide.  Can be protected or unprotected as shown in specification.
KERT

SEQ ID NO: 325 - protein, 4 aa, Artificial
  Synthetic peptide.  Can be protected or unprotected as shown in specification.
KDRS

SEQ ID NO: 326 - protein, 4 aa, Artificial
  Synthetic peptide.  Can be protected or unprotected as shown in specification.
KDRT

SEQ ID NO: 327 - protein, 4 aa, Artificial
  Synthetic peptide.  Can be protected or unprotected as shown in specification.
KRES

SEQ ID NO: 328 - protein, 4 aa, Artificial
  Synthetic peptide.  Can be protected or unprotected as shown in specification.
KRET

SEQ ID NO: 329 - protein, 4 aa, Artificial
  Synthetic peptide.  Can be protected or unprotected as shown in specification.
KERL

SEQ ID NO: 330 - protein, 4 aa, Artificial
  Synthetic peptide.  Can be protected or unprotected as shown in specification.
XRES

SEQ ID NO: 331 - protein, 4 aa, Artificial
  Synthetic peptide.  Can be protected or unprotected as shown in specification.
XERS

SEQ ID NO: 332 - protein, 4 aa, Artificial
  Synthetic peptide.  Can be protected or unprotected as shown in specification.
XRDS

SEQ ID NO: 333 - protein, 4 aa, Artificial
  Synthetic peptide.  Can be protected or unprotected as shown in specification.
XDRS

SEQ ID NO: 334 - protein, 4 aa, Artificial
  Synthetic peptide.  Can be protected or unprotected as shown in specification.
XDRT

SEQ ID NO: 335 - protein, 4 aa, Artificial
  Synthetic peptide.  Can be protected or unprotected as shown in specification.
XRDT

SEQ ID NO: 336 - protein, 4 aa, Artificial
  Synthetic peptide.  Can be protected or unprotected as shown in specification.
XERT

SEQ ID NO: 337 - protein, 4 aa, Artificial
  Synthetic peptide.  Can be protected or unprotected as shown in specification.
XRET

SEQ ID NO: 338 - protein, 4 aa, Artificial
  Synthetic peptide.  Can be protected or unprotected as shown in specification.
WDRI

SEQ ID NO: 339 - protein, 4 aa, Artificial
  Synthetic peptide.  Can be protected or unprotected as shown in specification.
WREI

SEQ ID NO: 340 - protein, 4 aa, Artificial
  Synthetic peptide.  Can be protected or unprotected as shown in specification.
WERI

SEQ ID NO: 341 - protein, 4 aa, Artificial
  Synthetic peptide.  Can be protected or unprotected as shown in specification.
WDRL

SEQ ID NO: 342 - protein, 4 aa, Artificial
  Synthetic peptide.  Can be protected or unprotected as shown in specification.
WREL

SEQ ID NO: 343 - protein, 4 aa, Artificial
  Synthetic peptide.  Can be protected or unprotected as shown in specification.
WERL

SEQ ID NO: 344 - protein, 4 aa, Artificial
  Synthetic peptide.  Can be protected or unprotected as shown in specification.
FDRI

SEQ ID NO: 345 - protein, 4 aa, Artificial
  Synthetic peptide.  Can be protected or unprotected as shown in specification.
FREI

SEQ ID NO: 346 - protein, 4 aa, Artificial
  Synthetic peptide.  Can be protected or unprotected as shown in specification.
FERI

SEQ ID NO: 347 - protein, 4 aa, Artificial
  Synthetic peptide.  Can be protected or unprotected as shown in specification.
FDRL

SEQ ID NO: 348 - protein, 4 aa, Artificial
  Synthetic peptide.  Can be protected or unprotected as shown in specification.
FREL

SEQ ID NO: 349 - protein, 4 aa, Artificial
  Synthetic peptide.  Can be protected or unprotected as shown in specification.
FERL

SEQ ID NO: 350 - protein, 4 aa, Artificial
  Synthetic peptide.  Can be protected or unprotected as shown in specification.
WRDF

SEQ ID NO: 351 - protein, 4 aa, Artificial
  Synthetic peptide.  Can be protected or unprotected as shown in specification.
WREF

SEQ ID NO: 352 - protein, 4 aa, Artificial
  Synthetic peptide.  Can be protected or unprotected as shown in specification.
WERF

SEQ ID NO: 353 - protein, 4 aa, Artificial
  Synthetic peptide.  Can be protected or unprotected as shown in specification.
WDRY

SEQ ID NO: 354 - protein, 4 aa, Artificial
  Synthetic peptide.  Can be protected or unprotected as shown in specification.
WREY

SEQ ID NO: 355 - protein, 4 aa, Artificial
  Synthetic peptide.  Can be protected or unprotected as shown in specification.
WERY

SEQ ID NO: 356 - protein, 4 aa, Artificial
  Synthetic peptide.  Can be protected or unprotected as shown in specification.
WRDT

SEQ ID NO: 357 - protein, 4 aa, Artificial
  Synthetic peptide.  Can be protected or unprotected as shown in specification.
WDRT

SEQ ID NO: 358 - protein, 4 aa, Artificial
  Synthetic peptide.  Can be protected or unprotected as shown in specification.
WRET

SEQ ID NO: 359 - protein, 4 aa, Artificial
  Synthetic peptide.  Can be protected or unprotected as shown in specification.
WERT

SEQ ID NO: 360 - protein, 4 aa, Artificial
  Synthetic peptide.  Can be protected or unprotected as shown in specification.
FRDX

SEQ ID NO: 361 - protein, 4 aa, Artificial
  Synthetic peptide.  Can be protected or unprotected as shown in specification.
FREX

SEQ ID NO: 362 - protein, 4 aa, Artificial
  Synthetic peptide.  Can be protected or unprotected as shown in specification.
FKDL

SEQ ID NO: 363 - protein, 4 aa, Artificial
  Synthetic peptide.  Can be protected or unprotected as shown in specification.
FDKL

SEQ ID NO: 364 - protein, 4 aa, Artificial
  Synthetic peptide.  Can be protected or unprotected as shown in specification.
FKEL

SEQ ID NO: 365 - protein, 4 aa, Artificial
  Synthetic peptide.  Can be protected or unprotected as shown in specification.
FEKL

SEQ ID NO: 366 - protein, 4 aa, Artificial
  Synthetic peptide.  Can be protected or unprotected as shown in specification.
FKDI

SEQ ID NO: 367 - protein, 4 aa, Artificial
  Synthetic peptide.  Can be protected or unprotected as shown in specification.
FDKI

SEQ ID NO: 368 - protein, 4 aa, Artificial
  Synthetic peptide.  Can be protected or unprotected as shown in specification.
FKEI

SEQ ID NO: 369 - protein, 4 aa, Artificial
  Synthetic peptide.  Can be protected or unprotected as shown in specification.
FEKI

SEQ ID NO: 370 - protein, 4 aa, Artificial
  Synthetic peptide.  Can be protected or unprotected as shown in specification.
FKDX

SEQ ID NO: 371 - protein, 4 aa, Artificial
  Synthetic peptide.  Can be protected or unprotected as shown in specification.
FDKX

SEQ ID NO: 372 - protein, 4 aa, Artificial
  Synthetic peptide.  Can be protected or unprotected as shown in specification.
FKEX

SEQ ID NO: 373 - protein, 4 aa, Artificial
  Synthetic peptide.  Can be protected or unprotected as shown in specification.
FEKX

SEQ ID NO: 374 - protein, 4 aa, Artificial
  Synthetic peptide.  Can be protected or unprotected as shown in specification.
FHDL

SEQ ID NO: 375 - protein, 4 aa, Artificial
  Synthetic peptide.  Can be protected or unprotected as shown in specification.
FDHL

SEQ ID NO: 376 - protein, 4 aa, Artificial
  Synthetic peptide.  Can be protected or unprotected as shown in specification.
FHEL

SEQ ID NO: 377 - protein, 4 aa, Artificial
  Synthetic peptide.  Can be protected or unprotected as shown in specification.
FEHL

SEQ ID NO: 378 - protein, 4 aa, Artificial
  Synthetic peptide.  Can be protected or unprotected as shown in specification.
FHDI

SEQ ID NO: 379 - protein, 4 aa, Artificial
  Synthetic peptide.  Can be protected or unprotected as shown in specification.
FDHI

SEQ ID NO: 380 - protein, 4 aa, Artificial
  Synthetic peptide.  Can be protected or unprotected as shown in specification.
FHEI

SEQ ID NO: 381 - protein, 4 aa, Artificial
  Synthetic peptide.  Can be protected or unprotected as shown in specification.
FEHI

SEQ ID NO: 382 - protein, 4 aa, Artificial
  Synthetic peptide.  Can be protected or unprotected as shown in specification.
FHDX

SEQ ID NO: 383 - protein, 4 aa, Artificial
  Synthetic peptide.  Can be protected or unprotected as shown in specification.
FDHX

SEQ ID NO: 384 - protein, 4 aa, Artificial
  Synthetic peptide.  Can be protected or unprotected as shown in specification.
FHEX

SEQ ID NO: 385 - protein, 4 aa, Artificial
  Synthetic peptide.  Can be protected or unprotected as shown in specification.
FEHX

SEQ ID NO: 386 - protein, 4 aa, Artificial
  Synthetic peptide.  Can be protected or unprotected as shown in specification.
KKDS

SEQ ID NO: 387 - protein, 4 aa, Artificial
  Synthetic peptide.  Can be protected or unprotected as shown in specification.
KDKS

SEQ ID NO: 388 - protein, 4 aa, Artificial
  Synthetic peptide.  Can be protected or unprotected as shown in specification.
KKES

SEQ ID NO: 389 - protein, 4 aa, Artificial
  Synthetic peptide.  Can be protected or unprotected as shown in specification.
KEKS

SEQ ID NO: 390 - protein, 4 aa, Artificial
  Synthetic peptide.  Can be protected or unprotected as shown in specification.
KHDS

SEQ ID NO: 391 - protein, 4 aa, Artificial
  Synthetic peptide.  Can be protected or unprotected as shown in specification.
KDHS

SEQ ID NO: 392 - protein, 4 aa, Artificial
  Synthetic peptide.  Can be protected or unprotected as shown in specification.
KHES

SEQ ID NO: 393 - protein, 4 aa, Artificial
  Synthetic peptide.  Can be protected or unprotected as shown in specification.
KEHS

SEQ ID NO: 394 - protein, 4 aa, Artificial
  Synthetic peptide.  Can be protected or unprotected as shown in specification.
KLRS

SEQ ID NO: 395 - protein, 4 aa, Artificial
  Synthetic peptide.  Can be protected or unprotected as shown in specification.
KRLS

SEQ ID NO: 396 - protein, 4 aa, Artificial
  Synthetic peptide.  Can be protected or unprotected as shown in specification.
KLRT

SEQ ID NO: 397 - protein, 4 aa, Artificial
  Synthetic peptide.  Can be protected or unprotected as shown in specification.
KRLT

SEQ ID NO: 398 - protein, 4 aa, Artificial
  Synthetic peptide.  Can be protected or unprotected as shown in specification.
KELS

SEQ ID NO: 399 - protein, 4 aa, Artificial
  Synthetic peptide.  Can be protected or unprotected as shown in specification.
KLES

SEQ ID NO: 400 - protein, 4 aa, Artificial
  Synthetic peptide.  Can be protected or unprotected as shown in specification.
KELT

SEQ ID NO: 401 - protein, 4 aa, Artificial
  Synthetic peptide.  Can be protected or unprotected as shown in specification.
KLRS

SEQ ID NO: 402 - protein, 4 aa, Artificial
  Synthetic peptide.  Can be protected or unprotected as shown in specification.
KLRT

SEQ ID NO: 403 - protein, 4 aa, Artificial
  Synthetic peptide.  Can be protected or unprotected as shown in specification.
KELS

SEQ ID NO: 404 - protein, 4 aa, Artificial
  Synthetic peptide.  Can be protected or unprotected as shown in specification.
KELT

SEQ ID NO: 405 - protein, 4 aa, Artificial
  Synthetic peptide.  Can be protected or unprotected as shown in specification.
KEIT

SEQ ID NO: 406 - protein, 4 aa, Artificial
  Synthetic peptide.  Can be protected or unprotected as shown in specification.
KLRS

SEQ ID NO: 407 - protein, 4 aa, Artificial
  Synthetic peptide.  Can be protected or unprotected as shown in specification.
KLRT

SEQ ID NO: 408 - protein, 4 aa, Artificial
  Synthetic peptide.  Can be protected or unprotected as shown in specification.
KELS

SEQ ID NO: 409 - protein, 4 aa, Artificial
  Synthetic peptide.  Can be protected or unprotected as shown in specification.
KELT

SEQ ID NO: 410 - protein, 4 aa, Artificial
  Synthetic peptide.  Can be protected or unprotected as shown in specification.
KLRS

SEQ ID NO: 411 - protein, 4 aa, Artificial
  Synthetic peptide.  Can be protected or unprotected as shown in specification.
KRFT

SEQ ID NO: 412 - protein, 4 aa, Artificial
  Synthetic peptide.  Can be protected or unprotected as shown in specification.
KLRT

SEQ ID NO: 413 - protein, 4 aa, Artificial
  Synthetic peptide.  Can be protected or unprotected as shown in specification.
KEIT

SEQ ID NO: 414 - protein, 4 aa, Artificial
  Synthetic peptide.  Can be protected or unprotected as shown in specification.
KEVT

SEQ ID NO: 415 - protein, 4 aa, Artificial
  Synthetic peptide.  Can be protected or unprotected as shown in specification.
KEAT

SEQ ID NO: 416 - protein, 4 aa, Artificial
  Synthetic peptide.  Can be protected or unprotected as shown in specification.
KEGT

SEQ ID NO: 417 - protein, 4 aa, Artificial
  Synthetic peptide.  Can be protected or unprotected as shown in specification.
KELS

SEQ ID NO: 418 - protein, 4 aa, Artificial
  Synthetic peptide.  Can be protected or unprotected as shown in specification.
KELT

SEQ ID NO: 419 - protein, 4 aa, Artificial
  Synthetic peptide.  Can be protected or unprotected as shown in specification.
KRWY

SEQ ID NO: 420 - protein, 4 aa, Artificial
  Synthetic peptide.  Can be protected or unprotected as shown in specification.
KWRY

SEQ ID NO: 421 - protein, 4 aa, Artificial
  Synthetic peptide.  Can be protected or unprotected as shown in specification.
KRYW

SEQ ID NO: 422 - protein, 4 aa, Artificial
  Synthetic peptide.  Can be protected or unprotected as shown in specification.
KYRW

SEQ ID NO: 423 - protein, 5 aa, Artificial
  Synthetic peptide.  Can be protected or unprotected as shown in specification.
KRYWT

SEQ ID NO: 424 - protein, 4 aa, Artificial
  Synthetic peptide.  Can be protected or unprotected as shown in specification.
KRYT

SEQ ID NO: 425 - protein, 4 aa, Artificial
  Synthetic peptide.  Can be protected or unprotected as shown in specification.
KRWT

SEQ ID NO: 426 - protein, 4 aa, Artificial
  Synthetic peptide.  Can be protected or unprotected as shown in specification.
KRWY

SEQ ID NO: 427 - protein, 4 aa, Artificial
  Synthetic peptide.  Can be protected or unprotected as shown in specification.
KRYW

SEQ ID NO: 428 - protein, 5 aa, Artificial
  Synthetic peptide.  Can be protected or unprotected as shown in specification.
KRYWT

SEQ ID NO: 429 - protein, 4 aa, Artificial
  Synthetic peptide.  Can be protected or unprotected as shown in specification.
KRYT

SEQ ID NO: 430 - protein, 4 aa, Artificial
  Synthetic peptide.  Can be protected or unprotected as shown in specification.
KRWT

SEQ ID NO: 431 - protein, 4 aa, Artificial
  Synthetic peptide.  Can be protected or unprotected as shown in specification.
KRWY

SEQ ID NO: 432 - protein, 4 aa, Artificial
  Synthetic peptide.  Can be protected or unprotected as shown in specification.
KRYW

SEQ ID NO: 433 - protein, 5 aa, Artificial
  Synthetic peptide.  Can be protected or unprotected as shown in specification.
KRYWT

SEQ ID NO: 434 - protein, 4 aa, Artificial
  Synthetic peptide.  Can be protected or unprotected as shown in specification.
KRYT

SEQ ID NO: 435 - protein, 4 aa, Artificial
  Synthetic peptide.  Can be protected or unprotected as shown in specification.
KRWT

SEQ ID NO: 436 - protein, 4 aa, Artificial
  Synthetic peptide.  Can be protected or unprotected as shown in specification.
EKRY

SEQ ID NO: 437 - protein, 4 aa, Artificial
  Synthetic peptide.  Can be protected or unprotected as shown in specification.
KRWY

SEQ ID NO: 438 - protein, 4 aa, Artificial
  Synthetic peptide.  Can be protected or unprotected as shown in specification.
KRYW

SEQ ID NO: 439 - protein, 5 aa, Artificial
  Synthetic peptide.  Can be protected or unprotected as shown in specification.
KRYWT

SEQ ID NO: 440 - protein, 4 aa, Artificial
  Synthetic peptide.  Can be protected or unprotected as shown in specification.
KRYT

SEQ ID NO: 441 - protein, 4 aa, Artificial
  Synthetic peptide.  Can be protected or unprotected as shown in specification.
KRFT

SEQ ID NO: 442 - protein, 4 aa, Artificial
  Synthetic peptide.  Can be protected or unprotected as shown in specification.
KRWT

SEQ ID NO: 443 - protein, 5 aa, Artificial
  Synthetic peptide.  Can be protected or unprotected as shown in specification.
KFWFS

SEQ ID NO: 444 - protein, 5 aa, Artificial
  Synthetic peptide.  Can be protected or unprotected as shown in specification.
KFWFT

SEQ ID NO: 445 - protein, 5 aa, Artificial
  Synthetic peptide.  Can be protected or unprotected as shown in specification.
KFYFS

SEQ ID NO: 446 - protein, 5 aa, Artificial
  Synthetic peptide.  Can be protected or unprotected as shown in specification.
KFYFT

SEQ ID NO: 447 - protein, 5 aa, Artificial
  Synthetic peptide.  Can be protected or unprotected as shown in specification.
KFHFS

SEQ ID NO: 448 - protein, 5 aa, Artificial
  Synthetic peptide.  Can be protected or unprotected as shown in specification.
KFHFT

SEQ ID NO: 449 - protein, 6 aa, Artificial
  Synthetic peptide.  Can be protected or unprotected as shown in specification.
KVFFYS

SEQ ID NO: 450 - protein, 5 aa, Artificial
  Synthetic peptide.  Can be protected or unprotected as shown in specification.
KFWFS

SEQ ID NO: 451 - protein, 5 aa, Artificial
  Synthetic peptide.  Can be protected or unprotected as shown in specification.
KFWFT

SEQ ID NO: 452 - protein, 5 aa, Artificial
  Synthetic peptide.  Can be protected or unprotected as shown in specification.
KFYFS

SEQ ID NO: 453 - protein, 5 aa, Artificial
  Synthetic peptide.  Can be protected or unprotected as shown in specification.
KFYFT

SEQ ID NO: 454 - protein, 5 aa, Artificial
  Synthetic peptide.  Can be protected or unprotected as shown in specification.
KFHFS

SEQ ID NO: 455 - protein, 5 aa, Artificial
  Synthetic peptide.  Can be protected or unprotected as shown in specification.
KFHFT

SEQ ID NO: 456 - protein, 5 aa, Artificial
  Synthetic peptide.  Can be protected or unprotected as shown in specification.
LFWFT

SEQ ID NO: 457 - protein, 5 aa, Artificial
  Synthetic peptide.  Can be protected or unprotected as shown in specification.
LFWFS

SEQ ID NO: 458 - protein, 22 aa, Artificial
  Synthetic peptide.  Can be protected or unprotected as shown in specification.
LLEQLNEQFNWVSRLANLTQGE

SEQ ID NO: 459 - protein, 18 aa, Artificial
  Synthetic peptide.  Can be protected or unprotected as shown in specification.
LLEQLNEQFNWVSRLANL

SEQ ID NO: 460 - protein, 25 aa, Artificial
  Synthetic peptide.  Can be protected or unprotected as shown in specification.
NELQEMSNQGSKYVNKEIQNAVNGV

SEQ ID NO: 461 - protein, 21 aa, Artificial
  Synthetic peptide.  Can be protected or unprotected as shown in specification.
IQNAVNGVKQIKTLIEKTNEE

SEQ ID NO: 462 - protein, 32 aa, Artificial
  Synthetic peptide.  Can be protected or unprotected as shown in specification.
RKTLLSXAAEAKKKKEDALNETRESETKLKEL

SEQ ID NO: 463 - protein, 16 aa, Artificial
  Synthetic peptide.  Can be protected or unprotected as shown in specification.
PGVCNETMMALWEECK

SEQ ID NO: 464 - protein, 16 aa, Artificial
  Synthetic peptide.  Can be protected or unprotected as shown in specification.
PCLKQTCMKFYARVCR

SEQ ID NO: 465 - protein, 19 aa, Artificial
  Synthetic peptide.  Can be protected or unprotected as shown in specification.
ECKPCLKQTCMKFYARVCR

SEQ ID NO: 466 - protein, 10 aa, Artificial
  Synthetic peptide.  Can be protected or unprotected as shown in specification.
LVGRQLEEFL

SEQ ID NO: 467 - protein, 12 aa, Artificial
  Synthetic peptide.  Can be protected or unprotected as shown in specification.
MNGDRIDSLLEN

SEQ ID NO: 468 - protein, 11 aa, Artificial
  Synthetic peptide.  Can be protected or unprotected as shown in specification.
QQTHMLDVMQD

SEQ ID NO: 469 - protein, 14 aa, Artificial
  Synthetic peptide.  Can be protected or unprotected as shown in specification.
FSRASSIIDELFQD

SEQ ID NO: 470 - protein, 15 aa, Artificial
  Synthetic peptide.  Can be protected or unprotected as shown in specification.
PFLEMIHEAQQAMDI

SEQ ID NO: 471 - protein, 11 aa, Artificial
  Synthetic peptide.  Can be protected or unprotected as shown in specification.
PTEFIREGDDD

SEQ ID NO: 472 - protein, 15 aa, Artificial
  Synthetic peptide.  Can be protected or unprotected as shown in specification.
RMKDQCDKCREILSV

SEQ ID NO: 473 - protein, 32 aa, Artificial
  Synthetic peptide.  Can be protected or unprotected as shown in specification.
PSQAKLRRELDESLQVAERLTRKYNELLKSYQ

SEQ ID NO: 474 - protein, 22 aa, Artificial
  Synthetic peptide.  Can be protected or unprotected as shown in specification.
LLEQLNEQFNWVSRLANLTEGE

SEQ ID NO: 475 - protein, 11 aa, Artificial
  Synthetic peptide.  Can be protected or unprotected as shown in specification.
DQYYLRVTTVA

SEQ ID NO: 476 - protein, 14 aa, Artificial
  Synthetic peptide.  Can be protected or unprotected as shown in specification.
PSGVTEVVVKLFDS

SEQ ID NO: 477 - protein, 21 aa, Artificial
  Synthetic peptide.  Can be protected or unprotected as shown in specification.
PKFMETVAEKALQEYRKKHRE

SEQ ID NO: 478 - protein, 26 aa, Artificial
  Synthetic peptide.  Can be protected or unprotected as shown in specification.
WDRVKDLATVYVDVLKDSGRDYVSQF

SEQ ID NO: 479 - protein, 25 aa, Artificial
  Synthetic peptide.  Can be protected or unprotected as shown in specification.
VATVMWDYFSQLSNNAKEAVEHLQK

SEQ ID NO: 480 - protein, 27 aa, Artificial
  Synthetic peptide.  Can be protected or unprotected as shown in specification.
RWELALGRFWDYLRWVQTLSEQVQEEL

SEQ ID NO: 481 - protein, 35 aa, Artificial
  Synthetic peptide.  Can be protected or unprotected as shown in specification.
LSSQVTQELRALMDETMKELKELKAYKSELEEQLT

SEQ ID NO: 482 - protein, 26 aa, Artificial
  Synthetic peptide.  Can be protected or unprotected as shown in specification.
ARLSKELQAAQARLGADMEDVCGRLV

SEQ ID NO: 483 - protein, 26 aa, Artificial
  Synthetic peptide.  Can be protected or unprotected as shown in specification.
VRLASHLRKLRKRLLRDADDLQKRLA

SEQ ID NO: 484 - protein, 19 aa, Artificial
  Synthetic peptide.  Can be protected or unprotected as shown in specification.
PLVEDMQRQWAGLVEKVQA

SEQ ID NO: 485 - protein, 17 aa, Artificial
  Synthetic peptide.  Can be protected or unprotected as shown in specification.
MSTYTGIFTDQVLSVLK

SEQ ID NO: 486 - protein, 22 aa, Artificial
  Synthetic peptide.  Can be protected or unprotected as shown in specification.
LLSFMQGYMKHATKTAKDALSS

SEQ ID NO: 487 - protein, 17 aa, Artificial
  Synthetic peptide.  Can be protected or unprotected as shown in specification.
KWIYHLTEGSTDLRTEG

SEQ ID NO: 488 - protein, 17 aa, Artificial
  Synthetic peptide.  Can be protected or unprotected as shown in specification.
KWFYHLTEGSTDLRTEG

SEQ ID NO: 489 - protein, 17 aa, Artificial
  Synthetic peptide.  Can be protected or unprotected as shown in specification.
KWLYHLTEGSTDLRTEG

SEQ ID NO: 490 - protein, 17 aa, Artificial
  Synthetic peptide.  Can be protected or unprotected as shown in specification.
KWVYHLTEGSTDLRTEG

SEQ ID NO: 491 - protein, 17 aa, Artificial
  Synthetic peptide.  Can be protected or unprotected as shown in specification.
KYIWHLTEGSTDLRTEG

SEQ ID NO: 492 - protein, 17 aa, Artificial
  Synthetic peptide.  Can be protected or unprotected as shown in specification.
KWIYHFTEGSTDLRTEG

SEQ ID NO: 493 - protein, 17 aa, Artificial
  Synthetic peptide.  Can be protected or unprotected as shown in specification.
KWFYHITEGSTDLRTEG

SEQ ID NO: 494 - protein, 17 aa, Artificial
  Synthetic peptide.  Can be protected or unprotected as shown in specification.
KWLYHVTEGSTDLRTEG

SEQ ID NO: 495 - protein, 17 aa, Artificial
  Synthetic peptide.  Can be protected or unprotected as shown in specification.
KWVYHYTEGSTDLRTEG

SEQ ID NO: 496 - protein, 17 aa, Artificial
  Synthetic peptide.  Can be protected or unprotected as shown in specification.
KYIWHFTEGSTDLRTEG

SEQ ID NO: 497 - protein, 17 aa, Artificial
  Synthetic peptide.  Can be protected or unprotected as shown in specification.
KYIWHITEGSTDLRTEG

SEQ ID NO: 498 - protein, 17 aa, Artificial
  Synthetic peptide.  Can be protected or unprotected as shown in specification.
KYIWHVTEGSTDLRTEG

SEQ ID NO: 499 - protein, 17 aa, Artificial
  Synthetic peptide.  Can be protected or unprotected as shown in specification.
KYIWHYTEGSTDLRTEG

SEQ ID NO: 500 - protein, 17 aa, Artificial
  Synthetic peptide.  Can be protected or unprotected as shown in specification.
KFIWHLTEGSTDLRTEG

SEQ ID NO: 501 - protein, 17 aa, Artificial
  Synthetic peptide.  Can be protected or unprotected as shown in specification.
KLIWHLTEGSTDLRTEG

SEQ ID NO: 502 - protein, 17 aa, Artificial
  Synthetic peptide.  Can be protected or unprotected as shown in specification.
KIIWHLTEGSTDLRTEG

SEQ ID NO: 503 - protein, 17 aa, Artificial
  Synthetic peptide.  Can be protected or unprotected as shown in specification.
KYIWFLTEGSTDLRTEG

SEQ ID NO: 504 - protein, 17 aa, Artificial
  Synthetic peptide.  Can be protected or unprotected as shown in specification.
KWIYFLTEGSTDLRTEG

SEQ ID NO: 505 - protein, 17 aa, Artificial
  Synthetic peptide.  Can be protected or unprotected as shown in specification.
KWIYLLTEGSTDLRTEG

SEQ ID NO: 506 - protein, 17 aa, Artificial
  Synthetic peptide.  Can be protected or unprotected as shown in specification.
KWIYHFTEGSTDLRTEG

SEQ ID NO: 507 - protein, 17 aa, Artificial
  Synthetic peptide.  Can be protected or unprotected as shown in specification.
KWIYHYTEGSTDLRTEG

SEQ ID NO: 508 - protein, 17 aa, Artificial
  Synthetic peptide.  Can be protected or unprotected as shown in specification.
KWIYHITEGSTDLRTEG

SEQ ID NO: 509 - protein, 17 aa, Artificial
  Synthetic peptide.  Can be protected or unprotected as shown in specification.
KWIYHLSEGSTDLRTEG

SEQ ID NO: 510 - protein, 17 aa, Artificial
  Synthetic peptide.  Can be protected or unprotected as shown in specification.
KWIYHLTDGSTDLRTEG

SEQ ID NO: 511 - protein, 17 aa, Artificial
  Synthetic peptide.  Can be protected or unprotected as shown in specification.
KWIYHLTEGTSDLRTEG

SEQ ID NO: 512 - protein, 17 aa, Artificial
  Synthetic peptide.  Can be protected or unprotected as shown in specification.
KWIYHLTEGSTELRTEG

SEQ ID NO: 513 - protein, 17 aa, Artificial
  Synthetic peptide.  Can be protected or unprotected as shown in specification.
KWIYHLTEGSTDFRTEG

SEQ ID NO: 514 - protein, 17 aa, Artificial
  Synthetic peptide.  Can be protected or unprotected as shown in specification.
KWIYHLTEGSTDYRTEG

SEQ ID NO: 515 - protein, 17 aa, Artificial
  Synthetic peptide.  Can be protected or unprotected as shown in specification.
KWIYHLTEGSTDIRTEG

SEQ ID NO: 516 - protein, 17 aa, Artificial
  Synthetic peptide.  Can be protected or unprotected as shown in specification.
KWIYHLTEGSTDVRTEG

SEQ ID NO: 517 - protein, 17 aa, Artificial
  Synthetic peptide.  Can be protected or unprotected as shown in specification.
KWIYHLTEGSTDLKTEG

SEQ ID NO: 518 - protein, 17 aa, Artificial
  Synthetic peptide.  Can be protected or unprotected as shown in specification.
KWIYHLTEGSTDLRSEG

SEQ ID NO: 519 - protein, 17 aa, Artificial
  Synthetic peptide.  Can be protected or unprotected as shown in specification.
KWIYHLTEGSTDLRTDG

SEQ ID NO: 520 - protein, 17 aa, Artificial
  Synthetic peptide.  Can be protected or unprotected as shown in specification.
KWIYHLTEGSTDIKTEG

SEQ ID NO: 521 - protein, 17 aa, Artificial
  Synthetic peptide.  Can be protected or unprotected as shown in specification.
KWIYHLTEGSTDIRSEG

SEQ ID NO: 522 - protein, 17 aa, Artificial
  Synthetic peptide.  Can be protected or unprotected as shown in specification.
KWIYHLTEGSTDIKSEG

SEQ ID NO: 523 - protein, 17 aa, Artificial
  Synthetic peptide.  Can be protected or unprotected as shown in specification.
KWIYHLTEGSTDIKSDG

SEQ ID NO: 524 - protein, 17 aa, Artificial
  Synthetic peptide.  Can be protected or unprotected as shown in specification.
RWIYHLTEGSTDLRTEG

SEQ ID NO: 525 - protein, 17 aa, Artificial
  Synthetic peptide.  Can be protected or unprotected as shown in specification.
RYIWHLTEGSTDIRTEG

SEQ ID NO: 526 - protein, 17 aa, Artificial
  Synthetic peptide.  Can be protected or unprotected as shown in specification.
RWIYHLTEGSTDIRTDG

SEQ ID NO: 527 - protein, 17 aa, Artificial
  Synthetic peptide.  Can be protected or unprotected as shown in specification.
RWIFHLTEGSTDIRTEG

SEQ ID NO: 528 - protein, 17 aa, Artificial
  Synthetic peptide.  Can be protected or unprotected as shown in specification.
RWIYHLTEGSTDLKTEG

SEQ ID NO: 529 - protein, 17 aa, Artificial
  Synthetic peptide.  Can be protected or unprotected as shown in specification.
RWIYHLTDGSTDIRTEG

SEQ ID NO: 530 - protein, 17 aa, Artificial
  Synthetic peptide.  Can be protected or unprotected as shown in specification.
RWIYHLTDGSTDLRTEG

SEQ ID NO: 531 - protein, 17 aa, Artificial
  Synthetic peptide.  Can be protected or unprotected as shown in specification.
RWIYFLTEGSTDIRTEG

SEQ ID NO: 532 - protein, 17 aa, Artificial
  Synthetic peptide.  Can be protected or unprotected as shown in specification.
RWIYFLTEGSTDLRTEG

SEQ ID NO: 533 - protein, 17 aa, Artificial
  Synthetic peptide.  Can be protected or unprotected as shown in specification.
KWFYHLTEGSTDFRTEG

SEQ ID NO: 534 - protein, 17 aa, Artificial
  Synthetic peptide.  Can be protected or unprotected as shown in specification.
RWFYHLTEGSTDLRTEG

SEQ ID NO: 535 - protein, 17 aa, Artificial
  Synthetic peptide.  Can be protected or unprotected as shown in specification.
KWIFHLTEGSTDIRTDG

SEQ ID NO: 536 - protein, 17 aa, Artificial
  Synthetic peptide.  Can be protected or unprotected as shown in specification.
RWIYHLTEGSTDIRTDG

SEQ ID NO: 537 - protein, 17 aa, Artificial
  Synthetic peptide.  Can be protected or unprotected as shown in specification.
RWIYHLTEGSTDLRTDG

SEQ ID NO: 538 - protein, 17 aa, Artificial
  Synthetic peptide.  Can be protected or unprotected as shown in specification.
KWIYHLTEGSTDIKTEG

SEQ ID NO: 539 - protein, 17 aa, Artificial
  Synthetic peptide.  Can be protected or unprotected as shown in specification.
KWIYHLTEGSTDIKTDG

SEQ ID NO: 540 - protein, 17 aa, Artificial
  Synthetic peptide.  Can be protected or unprotected as shown in specification.
KWIYHLTEGSTDFKTEG

SEQ ID NO: 541 - protein, 17 aa, Artificial
  Synthetic peptide.  Can be protected or unprotected as shown in specification.
KWIYHLTEGSTDYKTEG

SEQ ID NO: 542 - protein, 17 aa, Artificial
  Synthetic peptide.  Can be protected or unprotected as shown in specification.
KWIYHLTEGSTDIRTEG

SEQ ID NO: 543 - protein, 17 aa, Artificial
  Synthetic peptide.  Can be protected or unprotected as shown in specification.
KWFYHFTEGSTDLRTEG

SEQ ID NO: 544 - protein, 17 aa, Artificial
  Synthetic peptide.  Can be protected or unprotected as shown in specification.
RWFYHFTEGSTDLRTEG

SEQ ID NO: 545 - protein, 17 aa, Artificial
  Synthetic peptide.  Can be protected or unprotected as shown in specification.
KWFYHFTEGSTDFRTEG

SEQ ID NO: 546 - protein, 17 aa, Artificial
  Synthetic peptide.  Can be protected or unprotected as shown in specification.
KWFYHFTDGSTDIRTEG

SEQ ID NO: 547 - protein, 17 aa, Artificial
  Synthetic peptide.  Can be protected or unprotected as shown in specification.
RWFYHFTEGSTDLRTEG

SEQ ID NO: 548 - protein, 17 aa, Artificial
  Synthetic peptide.  Can be protected or unprotected as shown in specification.
RWFYHFTEGSTDFRTEG

SEQ ID NO: 549 - protein, 17 aa, Artificial
  Synthetic peptide.  Can be protected or unprotected as shown in specification.
RWFYHFTEGSTDFRTDG

SEQ ID NO: 550 - protein, 19 aa, Artificial
  Synthetic peptide.  Can be protected or unprotected as shown in specification.
EKCVEEFKSLTSCLDSKAF

SEQ ID NO: 551 - protein, 19 aa, Artificial
  Synthetic peptide.  Can be protected or unprotected as shown in specification.
DKCVEEFKSLTSCLDSKAF

SEQ ID NO: 552 - protein, 19 aa, Artificial
  Synthetic peptide.  Can be protected or unprotected as shown in specification.
EKCVDEFKSLTSCLDSKAF

SEQ ID NO: 553 - protein, 19 aa, Artificial
  Synthetic peptide.  Can be protected or unprotected as shown in specification.
EKCVEDFKSLTSCLDSKAF

SEQ ID NO: 554 - protein, 19 aa, Artificial
  Synthetic peptide.  Can be protected or unprotected as shown in specification.
ERCVEEFKSLTSCLDSKAF

SEQ ID NO: 555 - protein, 19 aa, Artificial
  Synthetic peptide.  Can be protected or unprotected as shown in specification.
DKCVDDFKSLTSCLDSKAF

SEQ ID NO: 556 - protein, 19 aa, Artificial
  Synthetic peptide.  Can be protected or unprotected as shown in specification.
DRCVEEFKSLTSCLDSKAF

SEQ ID NO: 557 - protein, 19 aa, Artificial
  Synthetic peptide.  Can be protected or unprotected as shown in specification.
ERCVDDFKSLTSCLDSKAF

SEQ ID NO: 558 - protein, 19 aa, Artificial
  Synthetic peptide.  Can be protected or unprotected as shown in specification.
EKCVEEFKSFTSCLDSKAF

SEQ ID NO: 559 - protein, 19 aa, Artificial
  Synthetic peptide.  Can be protected or unprotected as shown in specification.
EKCVEEFKSITSCLDSKAF

SEQ ID NO: 560 - protein, 19 aa, Artificial
  Synthetic peptide.  Can be protected or unprotected as shown in specification.
EKCVEEFKSVTSCLDSKAF

SEQ ID NO: 561 - protein, 19 aa, Artificial
  Synthetic peptide.  Can be protected or unprotected as shown in specification.
ERCVEEFKSYTSCLDSKAF

SEQ ID NO: 562 - protein, 19 aa, Artificial
  Synthetic peptide.  Can be protected or unprotected as shown in specification.
ERCVEEFKSFTSCLDSKAF

SEQ ID NO: 563 - protein, 19 aa, Artificial
  Synthetic peptide.  Can be protected or unprotected as shown in specification.
ERCVEEFKSITSCLDSKAF

SEQ ID NO: 564 - protein, 19 aa, Artificial
  Synthetic peptide.  Can be protected or unprotected as shown in specification.
ERCVEEFKSVTSCLDSKAF

SEQ ID NO: 565 - protein, 19 aa, Artificial
  Synthetic peptide.  Can be protected or unprotected as shown in specification.
ERCVEEFKSYTSCLDSKAF

SEQ ID NO: 566 - protein, 19 aa, Artificial
  Synthetic peptide.  Can be protected or unprotected as shown in specification.
EKCVEEFKSFTTCLDSKAF

SEQ ID NO: 567 - protein, 19 aa, Artificial
  Synthetic peptide.  Can be protected or unprotected as shown in specification.
EKCVEEFKSISSCLDSKAF

SEQ ID NO: 568 - protein, 19 aa, Artificial
  Synthetic peptide.  Can be protected or unprotected as shown in specification.
EKCVEEFKSVSTCLDSKAF

SEQ ID NO: 569 - protein, 19 aa, Artificial
  Synthetic peptide.  Can be protected or unprotected as shown in specification.
EKCVEEFKSYTSCLDSKAF

SEQ ID NO: 570 - protein, 19 aa, Artificial
  Synthetic peptide.  Can be protected or unprotected as shown in specification.
EKCVEEFKSFTTCLDSKAF

SEQ ID NO: 571 - protein, 19 aa, Artificial
  Synthetic peptide.  Can be protected or unprotected as shown in specification.
EKCVEEFKSFSSCLDSKAF

SEQ ID NO: 572 - protein, 19 aa, Artificial
  Synthetic peptide.  Can be protected or unprotected as shown in specification.
EKCVEEFKSFTSCLDSKAF

SEQ ID NO: 573 - protein, 19 aa, Artificial
  Synthetic peptide.  Can be protected or unprotected as shown in specification.
EKCVEEFKSFTSCLDSKAF

SEQ ID NO: 574 - protein, 19 aa, Artificial
  Synthetic peptide.  Can be protected or unprotected as shown in specification.
EKCVEEFKSFTSCLDSKAF

SEQ ID NO: 575 - protein, 19 aa, Artificial
  Synthetic peptide.  Can be protected or unprotected as shown in specification.
EKCVEEFKSFTSCFDSKAF

SEQ ID NO: 576 - protein, 19 aa, Artificial
  Synthetic peptide.  Can be protected or unprotected as shown in specification.
EKCVEEFKSFTSCFESKAF

SEQ ID NO: 577 - protein, 19 aa, Artificial
  Synthetic peptide.  Can be protected or unprotected as shown in specification.
EKCVEEFKSFTSCLESKAF

SEQ ID NO: 578 - protein, 19 aa, Artificial
  Synthetic peptide.  Can be protected or unprotected as shown in specification.
EKCVEEFKSFTSCIDSKAF

SEQ ID NO: 579 - protein, 19 aa, Artificial
  Synthetic peptide.  Can be protected or unprotected as shown in specification.
EKCVEELKSFTSCFDSKAF

SEQ ID NO: 580 - protein, 19 aa, Artificial
  Synthetic peptide.  Can be protected or unprotected as shown in specification.
DKCVEEFKSFTSCFDSKAF

SEQ ID NO: 581 - protein, 19 aa, Artificial
  Synthetic peptide.  Can be protected or unprotected as shown in specification.
DKCVEEFKSFTSCFESKAF

SEQ ID NO: 582 - protein, 19 aa, Artificial
  Synthetic peptide.  Can be protected or unprotected as shown in specification.
ERCVEEFKSFTSCFDSKAF

SEQ ID NO: 583 - protein, 19 aa, Artificial
  Synthetic peptide.  Can be protected or unprotected as shown in specification.
EKCFEEFKSFTSCFDSKAF

SEQ ID NO: 584 - protein, 19 aa, Artificial
  Synthetic peptide.  Can be protected or unprotected as shown in specification.
EKCFEEFKSFTSCFESKAF

SEQ ID NO: 585 - protein, 19 aa, Artificial
  Synthetic peptide.  Can be protected or unprotected as shown in specification.
EKCVEEFKSFSSCFESKAF

SEQ ID NO: 586 - protein, 19 aa, Artificial
  Synthetic peptide.  Can be protected or unprotected as shown in specification.
EKCVEEFKSFQSCFDSKAF

SEQ ID NO: 587 - protein, 19 aa, Artificial
  Synthetic peptide.  Can be protected or unprotected as shown in specification.
EKCFEEFKSFQSCFDSKAF

SEQ ID NO: 588 - protein, 19 aa, Artificial
  Synthetic peptide.  Can be protected or unprotected as shown in specification.
EKCVEEFKQFTSCFDSKAF

SEQ ID NO: 589 - protein, 19 aa, Artificial
  Synthetic peptide.  Can be protected or unprotected as shown in specification.
EKCVEEFKQLTSCLDSKAF

SEQ ID NO: 590 - protein, 19 aa, Artificial
  Synthetic peptide.  Can be protected or unprotected as shown in specification.
EKCFEEFKSFQSCLDSKAF

SEQ ID NO: 591 - protein, 19 aa, Artificial
  Synthetic peptide.  Can be protected or unprotected as shown in specification.
EKCVEEFKQFTSCFDSKAF

SEQ ID NO: 592 - protein, 19 aa, Artificial
  Synthetic peptide.  Can be protected or unprotected as shown in specification.
EKCVEEFKSFTSCFESKAF

SEQ ID NO: 593 - protein, 19 aa, Artificial
  Synthetic peptide.  Can be protected or unprotected as shown in specification.
ERCFEEFKSFTSCFDSKAF

SEQ ID NO: 594 - protein, 19 aa, Artificial
  Synthetic peptide.  Can be protected or unprotected as shown in specification.
DKCFEEFKSFTSCFDSKAF

SEQ ID NO: 595 - protein, 19 aa, Artificial
  Synthetic peptide.  Can be protected or unprotected as shown in specification.
ERCVEEFKSLTSCLESKAF

SEQ ID NO: 596 - protein, 19 aa, Artificial
  Synthetic peptide.  Can be protected or unprotected as shown in specification.
EKCVEEFKSLTSCLDSKFF

SEQ ID NO: 597 - protein, 19 aa, Artificial
  Synthetic peptide.  Can be protected or unprotected as shown in specification.
EKCFEEFKSFTSCFDSKFF

SEQ ID NO: 598 - protein, 19 aa, Artificial
  Synthetic peptide.  Can be protected or unprotected as shown in specification.
DKCFEEFKSFTSCLDSKFF

SEQ ID NO: 599 - protein, 19 aa, Artificial
  Synthetic peptide.  Can be protected or unprotected as shown in specification.
DKCFEEFKSFTSCLESKFF

SEQ ID NO: 600 - protein, 19 aa, Artificial
  Synthetic peptide.  Can be protected or unprotected as shown in specification.
DKCFEELKSFTSCLDSKFF

SEQ ID NO: 601 - protein, 19 aa, Artificial
  Synthetic peptide.  Can be protected or unprotected as shown in specification.
ERCFEEFKSFTSCLDSKFF

SEQ ID NO: 602 - protein, 19 aa, Artificial
  Synthetic peptide.  Can be protected or unprotected as shown in specification.
EKAVEEFKSFTSCLDSKAF

SEQ ID NO: 603 - protein, 19 aa, Artificial
  Synthetic peptide.  Can be protected or unprotected as shown in specification.
DKAVEEFKSFTSCLDSKFF

SEQ ID NO: 604 - protein, 19 aa, Artificial
  Synthetic peptide.  Can be protected or unprotected as shown in specification.
EKAVEEFKSFTSALDSKAF

SEQ ID NO: 605 - protein, 19 aa, Artificial
  Synthetic peptide.  Can be protected or unprotected as shown in specification.
DKAVEEFKSFTSALDSKAF

SEQ ID NO: 606 - protein, 19 aa, Artificial
  Synthetic peptide.  Can be protected or unprotected as shown in specification.
DRAFEEFKSFTSCLDSKFF

SEQ ID NO: 607 - protein, 19 aa, Artificial
  Synthetic peptide.  Can be protected or unprotected as shown in specification.
DRAFEEFKSFTSALDSKFF

SEQ ID NO: 608 - protein, 19 aa, Artificial
  Synthetic peptide.  Can be protected or unprotected as shown in specification.
DKCFEEFKSFTSCFESKFF

SEQ ID NO: 609 - protein, 19 aa, Artificial
  Synthetic peptide.  Can be protected or unprotected as shown in specification.
EKCYEEFKSFTSCLDSKFF

SEQ ID NO: 610 - protein, 19 aa, Artificial
  Synthetic peptide.  Can be protected or unprotected as shown in specification.
DKCWEEFKSFTSCLDSKFF

SEQ ID NO: 611 - protein, 19 aa, Artificial
  Synthetic peptide.  Can be protected or unprotected as shown in specification.
EKCFEEFKSYTSCLDSKFF

SEQ ID NO: 612 - protein, 19 aa, Artificial
  Synthetic peptide.  Can be protected or unprotected as shown in specification.
EKCFEEFKSWTSCLDSKFF

SEQ ID NO: 613 - protein, 19 aa, Artificial
  Synthetic peptide.  Can be protected or unprotected as shown in specification.
EKCVEEFKSWTSCLDSKAF

SEQ ID NO: 614 - protein, 19 aa, Artificial
  Synthetic peptide.  Can be protected or unprotected as shown in specification.
DKCFEEFKSWTSCLDSKAF

SEQ ID NO: 615 - protein, 18 aa, Artificial
  Synthetic peptide.  Can be protected or unprotected as shown in specification.
DVWKAAYDKFAEKFKEFF

SEQ ID NO: 616 - protein, 18 aa, Artificial
  Synthetic peptide.  Can be protected or unprotected as shown in specification.
DVWKAFYDKFAEKFKEAF

SEQ ID NO: 617 - protein, 18 aa, Artificial
  Synthetic peptide.  Can be protected or unprotected as shown in specification.
DFWKAFYDKVAEKFKEAF

SEQ ID NO: 618 - protein, 18 aa, Artificial
  Synthetic peptide.  Can be protected or unprotected as shown in specification.
FFEKFKEAFKDYAAKWVD

SEQ ID NO: 619 - protein, 18 aa, Artificial
  Synthetic peptide.  Can be protected or unprotected as shown in specification.
FAEKFKEAFKDYFAKWVD

SEQ ID NO: 620 - protein, 18 aa, Artificial
  Synthetic peptide.  Can be protected or unprotected as shown in specification.
FAEKFKEAVKDYFAKWFD

SEQ ID NO: 621 - protein, 7 aa, Artificial
  Synthetic peptide linker.
GGGGSSS

SEQ ID NO: 622 - protein, 45 aa, Artificial [sequence omitted]

SEQ ID NO: 623 - protein, 41 aa, Artificial
  Synthetic peptide.  Can be protected or unprotected as shown in specification.
LLEQLNEQFNWVSRLANLTQGEPDWFKAFYDKVAEKFKEAF

SEQ ID NO: 624 - protein, 7 aa, Artificial
  Synthetic peptide linker.
GGGGSSS

SEQ ID NO: 625 - protein, 10 aa, Artificial
  Synthetic peptide.  Can be protected or unprotected as shown in specification.
LVGRQLEEFL

SEQ ID NO: 626 - protein, 22 aa, Artificial
  Synthetic peptide.  Can be protected or unprotected as shown in specification.
LLEQLNEQFNWVSRLANLTQGE

SEQ ID NO: 627 - protein, 13 aa, Artificial
  Synthetic peptide.  Can be protected or unprotected as shown in specification.
SGVTEVVVKLFDS

SEQ ID NO: 628 - protein, 11 aa, Artificial
  Synthetic peptide.  Can be protected or unprotected as shown in specification.
QQTHMLDVMQD

SEQ ID NO: 629 - protein, 6 aa, Artificial
  Synthetic peptide.  Can be protected or unprotected as shown in specification.
KFKEAF

SEQ ID NO: 630 - protein, 6 aa, Artificial
  Synthetic peptide.  Can be protected or unprotected as shown in specification.
YDKVAE

SEQ ID NO: 631 - protein, 6 aa, Artificial
  Synthetic peptide.  Can be protected or unprotected as shown in specification.
DWFKAF

SEQ ID NO: 632 - protein, 6 aa, Artificial
  Synthetic peptide.  Can be protected or unprotected as shown in specification.
KFKEAF

SEQ ID NO: 633 - protein, 6 aa, Artificial
  Synthetic peptide.  Can be protected or unprotected as shown in specification.
KFKEAF

SEQ ID NO: 634 - protein, 11 aa, Artificial
  Synthetic peptide.  Can be protected or unprotected as shown in specification.
YDKVAEKFKEF

SEQ ID NO: 635 - protein, 18 aa, Artificial
  Synthetic peptide.  Can be protected or unprotected as shown in specification.
DWFKAFYDKVAEKFKEAF

SEQ ID NO: 636 - protein, 6 aa, Artificial
  Synthetic peptide.  Can be protected or unprotected as shown in specification.
DWFKAF

SEQ ID NO: 637 - protein, 6 aa, Artificial
  Synthetic peptide.  Can be protected or unprotected as shown in specification.
YDKVAE

SEQ ID NO: 638 - protein, 6 aa, Artificial
  Synthetic peptide.  Can be protected or unprotected as shown in specification.
KFKEAF

SEQ ID NO: 639 - protein, 6 aa, Artificial
  Synthetic peptide.  Can be protected or unprotected as shown in specification.
DWFKAF

SEQ ID NO: 640 - protein, 6 aa, Artificial
  Synthetic peptide.  Can be protected or unprotected as shown in specification.
DWFKAF

SEQ ID NO: 641 - protein, 6 aa, Artificial
  Synthetic peptide.  Can be protected or unprotected as shown in specification.
YDKVAE

SEQ ID NO: 642 - protein, 6 aa, Artificial
  Synthetic peptide.  Can be protected or unprotected as shown in specification.
KFKEAF

SEQ ID NO: 643 - protein, 17 aa, Artificial
  Synthetic peptide.  Can be protected or unprotected as shown in specification.
KWIYHLTEGSTDLRTEG

SEQ ID NO: 644 - protein, 7 aa, Artificial
  Synthetic peptide.  Can be protected or unprotected as shown in specification.
KAHYEAL

(19) United States
(12) Patent Application Publication
(10) Pub. No.: US 2012/0004720 A1
(43) Pub. Date: Jan. 5, 2012

(54) G-TYPE PEPTIDES AND OTHER AGENTS TO AMELIORATE ATHEROSCLEROSIS AND OTHER PATHOLOGIES

(75) Inventors: Alan M. Fogelman, Beverly Hills, CA (US); Mohamad Navab, Los Angeles, CA (US); Gattadahalli M. Anantharamaiah, Birmingham, AL (US)

(73) Assignees: THE UNIVERSITY OF ALABAMA, Tuscaloosa, AL (US); THE REGENTS OF THE UNIVERSITY OF CALIFORNIA, Oakland, CA (US)

(21) Appl. No.: 13/156,269

(22) Filed: Jun. 8, 2011

Related U.S. Application Data
(63) Continuation of application No. 11/229,042, filed on Sep. 16, 2005, now abandoned.
(60) Provisional application No. 60/610,711, filed on Sep. 16, 2004.

(51) Int. Cl. A61F 2/82; C07K 5/087; C07K 5/107; A61P 9/10; A61K 38/10; A61P 3/10; A61P 7/00; C07K 7/06; C07K 7/08

(52) U.S. Cl. 623/1.42; 530/329; 530/331; 530/330; 530/328; 530/326; 514/1.9; 514/6.9; 514/13.5

(57) ABSTRACT

This invention provides novel peptides, and other agents, that ameliorate one or more symptoms of atherosclerosis and/or other pathologies characterized by an inflammatory response. In certain embodiment, the peptides resemble a G* amphipathic helix of apolipoprotein J. The peptides are highly stable and readily administered via an oral route.

### CROSS-REFERENCE TO RELATED APPLICATIONS

This application is a Continuation of Ser. No. 11/229,042, filed on Sep. 16, 2005, which claims priority to and benefit of 60/610,711, filed on Sep. 16, 2004, both of which are incorporated herein by reference in its entirety for all purposes.

### STATEMENT AS TO RIGHTS TO INVENTIONS MADE UNDER FEDERALLY SPONSORED RESEARCH AND DEVELOPMENT

This work was supported, in part, by Grant No: HL30568 from the National Heart Blood Lung Institute of the National Institutes of Health. The Government of the United States of America may have certain rights in this invention.

### FIELD OF THE INVENTION

This invention relates to the field of atherosclerosis. In particular, this invention pertains to the identification of a class of peptides that are orally administrable and that ameliorate one or more symptoms of atherosclerosis or other pathologies characterized by an inflammatory response.

### BACKGROUND OF THE INVENTION

The introduction of statins (e.g. Mevacor®, Lipitor®) has reduced mortality from heart attack and stroke by about one-third. However, heart attack and stroke remain the major cause of death and disability, particularly in the United States and in Western European countries. Heart attack and stroke are the result of a chronic inflammatory condition, which is called atherosclerosis.

Several causative factors are implicated in the development of cardiovascular disease including hereditary predisposition to the disease, gender, lifestyle factors such as smoking and diet, age, hypertension, and hyperlipidemia, including hypercholesterolemia. Several of these factors, particularly hyperlipidemia and hypercholesteremia (high blood cholesterol concentrations) provide a significant risk factor associated with atherosclerosis.

Cholesterol is present in the blood as free and esterified cholesterol within lipoprotein particles, commonly known as chylomicrons, very low density lipoproteins (VLDLs), low density lipoproteins (LDLs), and high density lipoproteins (HDLs). Concentration of total cholesterol in the blood is influenced by (1) absorption of cholesterol from the digestive tract, (2) synthesis of cholesterol from dietary constituents such as carbohydrates, proteins, fats and ethanol, and (3) removal of cholesterol from blood by tissues, especially the liver, and subsequent conversion of the cholesterol to bile acids, steroid hormones, and biliary cholesterol.

Maintenance of blood cholesterol concentrations is influenced by both genetic and environmental factors. Genetic factors include concentration of rate-limiting enzymes in cholesterol biosynthesis, concentration of receptors for low density lipoproteins in the liver, concentration of rate-limiting enzymes for conversion of cholesterols bile acids, rates of synthesis and secretion of lipoproteins and gender of person. Environmental factors influencing the hemostasis of blood cholesterol concentration in humans include dietary composition, incidence of smoking, physical activity, and use of a variety of pharmaceutical agents. Dietary variables include amount and type of fat (saturated and polyunsaturatead fatty acids), amount of cholesterol, amount and type of fiber, and perhaps amounts of vitamins such as vitamin C and D and minerals such as calcium.

Low density lipoprotein (LDL) oxidation has been strongly implicated in the pathogenesis of atherosclerosis. High density lipoprotein (HDL) has been found to be capable of protecting against LDL oxidation, but in some instances has been found to accelerate LDL oxidation. Important initiating factors in atherosclerosis include the production of LDL-derived oxidized phospholipids.

Normal HDL has the capacity to prevent the formation of these oxidized phospholipids and also to inactivate these oxidized phospholipids once they have formed. However, under some circumstances HDL can be converted from an anti-inflammatory molecule to a pro-inflammatory molecule that actually promotes the formation of these oxidized phospholipids.

HDL and LDL have been suggested to be part of the innate immune system (Navab et al. (2001) Arterioscler Thromb Vasc Biol. 21: 481-488). The generation of anti-inflammatory HDL has been achieved with class A amphipathic helical peptides that mimic the major protein of HDL, apolipoprotein A-I (apo A-I) (see, e.g., WO 02/15923).

### SUMMARY OF THE INVENTION

This invention provides novel compositions and methods to ameliorate symptoms of atherosclerosis and other inflammatory conditions such as rheumatoid arthritis, lupus erythematous, polyarteritis nodosa, osteoporosis, Altzheimer's disease and viral illnesses such as influenza A.

In certain embodiments this invention provides “isolated” polypeptides that ameliorate a symptom of atherosclerosis or other pathologies associated with an inflammatory response and/or compositions comprising such polypeptides.

Thus, in one embodiment, this invention provides a peptide that ameliorates one or more symptoms of an inflammatory condition, where the peptide comprises the amino acid sequence LAEYHAK (SEQ ID NO: 2) or KAHYEAL (SEQ ID NO:638); and the peptide comprises at least one D amino acid and/or at least one protecting group. In certain embodiments the peptide comprises D amino acids and/or one or more protecting groups (e.g., a protecting group at each terminus). In various embodiments the protecting group(s) include onre or more protecting groups from the group consisting of amide, 3 to 20 carbon alkyl groups, Fmoc, t-boc, 9-fluoreneacetyl group, 1-fluorenecarboxylic group, 9-fluorenecarboxylic group, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), Benzyloxy (BzlO), Benzyl (Bzl), Benzoyl (Bz), 3-nitro-2-pyridinesulphenyl (Npys), 1-(4,4-dimethyl-2,6-dioxocyclohexylidene)ethyl (Dde), 2,6-dichlorobenzyl (2,6-DiCl-Bzl), 2-chlorobenzyloxycarbonyl (2-Cl—Z), 2-bromobenzyloxycarbonyl (2-Br—Z), Benzyloxymethyl (Bom), cyclohexyloxy (cHxO), t-butoxymethyl (Bum), t-butoxy (tBuO), t-Butyl (tBu), Acetyl (Ac), a propyl group, a butyl group, a pentyl group, a hexyl group, N-methyl anthranilyl, a polyethylene glycol (PEG), and Trifluoroacetyl (TFA).

In certain embodiments this invention provides a peptide that ameliorates one or more symptoms of an inflammatory condition, where the peptide: ranges in length from about 3 to about 10 amino acids; comprises an amino acid sequence where the sequence comprises acidic or basic amino acids alternating with one or two aromatic, hydrophobic, or uncharged polar amino acids; comprises hydrophobic terminal amino acids or terminal amino acids bearing a hydrophobic protecting group; and is not the sequence LAEYHAK (SEQ ID NO: 2) comprising all L amino acids; where the peptide converts pro-inflammatory HDL to anti-inflammatory HDL or makes anti-inflammatory HDL more anti-inflammatory. The peptide can, optionally, comprise one or more D amino acids and/or one or more protecting groups, e.g., as described above.

In various embodiments this invention provides peptide that amelioriates one or more symptoms of an inflammatory condition, where the peptide comprises the amino acid sequence of a peptide found in, e.g., Tables 3 or 14, or a concatamer thereof. In certain embodiments the peptide at least one D amino acid, in certain embodiments the peptide comprises all D amino acids. In various embodiments the peptide additionally or alternatively comprises at least one protecting group (e.g. a protecting group at each terminus). Certain suitable protecting groups sinclude, but are not limited to amide, 3 to 20 carbon alkyl groups, Fmoc, t-boc, 9-fluoreneacetyl group, 1-fluorenecarboxylic group, 9-fluorenecarboxylic group, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), Benzyloxy (BzlO), Benzyl (Bzl), Benzoyl (Bz), 3-nitro-2-pyridinesulphenyl (Npys), 1-(4,4-dimethyl-2,6-dioxocyclohexylidene)ethyl (Dde), 2,6-dichlorobenzyl (2,6-DiCl-Bzl), 2-chlorobenzyloxycarbonyl (2-Cl-Z), 2-bromobenzyloxycarbonyl (2-Br—Z), Benzyloxymethyl (Bom), cyclohexyloxy (cHxO),t-butoxymethyl (Bum), t-butoxy (tBuO), t-Butyl (tBu), Acetyl (Ac), a propyl group, a butyl group, a pentyl group, a hexyl group, N-methyl anthranilyl, a polyethylene glycol (PEG), Trifluoroacetyl (TFA), and the like.

In certain embodiments this invention provides a peptide that ameliorates one or more symptoms of an inflammatory condition, where: the peptide comprises an amino acid sequence selected from the group consisting of DMT-Arg-Phe-Lys, (SEQ ID NO:1), DMT-Arg-Glu-Leu (SEQ ID NO:2), Lys-Phe-Arg-DMT (SEQ ID NO:3), and Leu-Glu-Arg-DMT (SEQ ID NO:4), where DMT is dimethyltyrosine. Again, the peptide can comprise at least one D almino acid and/or at least one protecting group, e.g. as described above. In certain embodiments the peptide is BocDimethyltyrosine-D-Arg-Phe-Lys(OtBu) (SEQ ID NO:5), or BocDimethyltyrosine-Arg-Glu-Leu(OtBu) (SEQ ID NO:6).

This invention also contemplates pharmaceutical formulations comprising any of the active agents (e.g. peptides, organic molecules, etc.) described herein and a pharmaceutically acceptable excipient. In certain embodiments the active agent is a peptide and the peptide is formulated as a time release formulation. In certain embodiments the formulation is formulated as a unit dosage formulation. In certain embodiments the formulation is formulated for administration by a route selected from the group consisting of oral administration, nasal administration, rectal administration, intraperitoneal injection, intravascular injection, subcutaneous injection, transcutaneous administration, inhalation administration, and intramuscular injection.

This invention also provides methods for the treatment or prophylaxis of a condition such as atherosclerosis, restenosis, a coronary complication associated with an acute phase response to an inflammation in a mammal, or diabetes, where the method comprises administering to a mammal in need thereof one or more of the active agents (e.g., peptides) described herein. In certain embodiments the active agent is in a pharmaceutically acceptable excipient (e.g., an excipient suitable for oral administration) and/or can be formulated as a unit dosage formulation. In various embodiments the administering comprises administering the active agent(s) by a route selected from the group consisting of oral administration, nasal administration, rectal administration, intraperitoneal injection, intravascular injection, subcutaneous injection, transcutaneous administration, and intramuscular injection. In various embodiments the mammal is a mammal (e.g. a human) diagnosed as having one or more symptoms of atherosclerosis, and/or diagnosed as at risk for stroke or atherosclerosis, and/or having or at risk for a coronary complication associated with an acute phase response to an inflammation, and/or having or being at risk for retenosis, and/or having or being at risk for diabetes.

Also provided is an active agent (e.g., a peptide) as described herein for use in the treatment of a condition selected from the group consisting of atherosclerosis, restenosis, a coronary complication associated with an acute phase response to an inflammation in a mammal, and diabetes. In certain embodiments this invention provides for the use of an active agent (e.g., a peptide) as described herein in the manufacture of a medicament for the therapeutic or prophylactic treatment of a condition selected from the group consisting of atherosclerosis, restenosis, a coronary complication associated with an acute phase response to an inflammation in a mammal, and diabetes.

In certain embodiments this invention also provides a stent for delivering drugs to a vessel in a body comprising: a stent framework including a plurality of reservoirs formed therein, and one or more active agents as described herein (e.g., in in Tables 1-15) and/or a small organic molecule as described herein positioned in the reservoirs. In various embodiments the active agent is a peptide comprising the amino acid sequence of 4F (SEQ ID NO:13). In various embodiments the active agent is contained within a polymer. In certain embodiments the stent framework comprises one of a metallic base or a polymeric base (e.g. a material such as stainless steel, nitinol, tantalum, MP35N alloy, platinum, titanium, a suitable biocompatible alloy, a suitable biocompatible polymer, and a combination thereof). The reservoirs can, optionally, comprise micropores and, In certain embodiments the micropores, when present, have a diameter of about 20 microns or less. In various embodiments the micropores, when present, have a diameter in the range of about 20 microns to about 50 microns. In various embodiments the micropores, when present, have a depth in the range of about 10 to about 50 microns. In various embodiments the micropores extend through the stent framework having an opening on an interior surface of the stent and an opening on an exterior surface of the stent. In certain embodiments the stent further comprises a cap layer disposed on the interior surface of the stent framework, the cap layer covering at least a portion of the through-holes and providing a barrier characteristic to control an elution rate of a drug in the drug polymer from the interior surface of the stent framework. In certain embodiments the reservoirs comprise channels along an exterior surface of the stent framework. In certain embodiments the polymer comprises a first layer of a first drug polymer having comprising a first active agent according to the present invention and the polymer layer comprises a second drug polymer having a active agent or other pharmaceutical. In various embodiments a barrier layer can be positioned between the polymer layers comprising the active agent(s) or on the surface of the polymer layer. In various embodiments a catheter is coupled to the stent framework. The catheter, can optionally comprise a means for expanding the stent, e.g., a balloon used to expand the stent, a sheath that retracts to allow expansion of the stent, and the like.

This invention also provides a method of manufacturing a drug-polymer stent, comprising: providing a stent framework; cutting a plurality of reservoirs in the stent framework; applying a composition comprising one or more of the active agents described herein to at least one reservoir; and drying the composition. The method can further optionally comprise applying a polymer layer to the dried composition; and drying the polymer layer.

In certain embodiments this invention provides a method of treating a vascular condition, comprising: positioning a stent (as described herein) within a vessel of a body; expanding the stent; and eluting at least one active agent from at least a surface of the stent.

Also provided are methods of synthesizing the various peptides described herein. In certain embodiments this invention provides a method of synthesizing a peptide, where the method comprises: providing at least 3 different peptide fragment subsequences of the peptide; and coupling the peptide fragment subsequences in solution phase to form the peptide. In certain embodiments the peptide ranges in length from 6 to 37 amino acids. In certain embodiments the peptide is 18 residues in length. In certain embodiments the peptide comprises a class A amphipathic helix. In various embodiments the peptide comprises the amino acid sequence D-W-F-K-A-F-Y-D-K-V-A-E-K-F-K-E-A-F (SEQ ID NO:13). In various embodiments all three peptide fragment subsequences are each 6 amino acids in length. In certain embodiments the three peptide fragment subsequences have the sequences: D-W-F-K-A-F (SEQ ID NO:641), Y-D-K-V-A-E (SEQ ID NO:642), and K-F-K-E-A-F (SEQ ID NO:643). In certain embodiments the peptide comprises all D amino acids.

### DEFINITIONS

The terms “isolated”, “purified”, or “biologically pure” when referring to an isolated polypeptide refer to material that is substantially or essentially free from components that normally accompany it as found in its native state. With respect to nucleic acids and/or polypeptides the term can refer to nucleic acids or polypeptides that are no longer flanked by the sequences typically flanking them in nature. Chemically synthesized polypeptides are “isolated” because they are not found in a native state (e.g. in blood, serum, etc.). In certain embodiments, the term “isolated” indicates that the polypeptide is not found in nature.

The terms “polypeptide”, “peptide” and “protein” are used interchangeably herein to refer to a polymer of amino acid residues. The terms apply to amino acid polymers in which one or more amino acid residues is an artificial chemical analogue of a corresponding naturally occurring amino acid, as well as to naturally occurring amino acid polymers.

The term “an amphipathic helical peptide” refers to a peptide comprising at least one amphipathic helix (amphipathic helical domain). Certain amphipathic helical peptides of this invention can comprise two or more (e.g. 3, 4, 5, etc.) amphipathic helices.

The term “class A amphipathic helix” refers to a protein structure that forms an α-helix producing a segregation of a polar and nonpolar faces with the positively charged residues residing at the polar-nonpolar interface and the negatively charged residues residing at the center of the polar face (see, e.g., “Segrest et al. (1990) Proteins: Structure, Function, and Genetics 8: 103-117).

“Apolipoprotein J” (apo J) is known by a variety of names including clusterin, TRPM2, GP80, and SP 40,40 (Fritz (1995) Pp 112 In: Clusterin: Role in Vertebrate Development, Function, and Adaptation (Harmony JAK Ed.), R. G. Landes, Georgetown, Tex.,). It was first described as a heterodimeric glycoprotein and a component of the secreted proteins of cultured rat Sertoli cells (Kissinger et al. (1982) Biol Reprod; 27:233240). The translated product is a single-chain precursor protein that undergoes intracellular cleavage into a disulfide-linked 34 kDa αsubunit and a 47 kDa βsubunit Collard and Griswold (187) Biochem., 26: 3297-3303). It has been associated with cellular injury, lipid transport, apoptosis and it may be involved in clearance of cellular debris caused by cell injury or death. Clusterin has been shown to bind to a variety of molecules with high affinity including lipids, peptides, and proteins and the hydrophobic probe 1-anilino-8-naphthalenesulfonate (Bailey et al. (2001) Biochem., 40: 11828-11840).

The class G amphipathic helix is found in globular proteins, and thus, the name class G. The feature of this class of amphipathic helix is that it possesses a random distribution of positively charged and negatively charged residues on the polar face with a narrow nonpolar face. Because of the narrow nonpolar face this class does not readily associate with phospholipid (see, Segrest et al. (1990) Proteins: Structure, Function, and Genetics. 8: 103-117; also see Erratum (1991) Proteins: Structure, Function and Genetics, 9: 79). Several exchangeable apolipoproteins possess similar but not identical characteristics to the G amphipathic helix. Similar to the class G amphipathic helix, this other class possesses a random distribution of positively and negatively charged residues on the polar face. However, in contrast to the class G amphipathic helix which has a narrow nonpolar face, this class has a wide nonpolar face that allows this class to readily bind phospholipid and the class is termed G* to differentiate it from the G class of amphipathic helix (see Segrest et al. (1992) J. Lipid Res., 33: 141-166; also see Anantharamaiah et al. (1993) Pp. 109-142 In: The Amphipathic Helix, Epand, R. M. Ed CRC Press, Boca Raton, Fla.). Computer programs to identify and classify amphipathic helical domains have been described by Jones et al. (1992) J. Lipid Res. 33: 287-296) and include, but are not limited to the helical wheel program (WHEEL or WHEEL/SNORKEL), helical net program (HELNET, HELNET/SNORKEL, HELNET/Angle), program for addition of helical wheels (COMBO or COMBO/SNORKEL), program for addition of helical nets (COMNET, COMNET/SNORKEL, COMBO/SELECT, COMBO/NET), consensus wheel program (CONSENSUS, CONSENSUS/SNORKEL), and the like.

The term “ameliorating” when used with respect to “ameliorating one or more symptoms of atherosclerosis” refers to a reduction, prevention, or elimination of one or more symptoms characteristic of atherosclerosis and/or associated pathologies. Such a reduction includes, but is not limited to a reduction or elimination of oxidized phospholipids, a reduction in atherosclerotic plaque formation and rupture, a reduction in clinical events such as heart attack, angina, or stroke, a decrease in hypertension, a decrease in inflammatory protein biosynthesis, reduction in plasma cholesterol, and the like.

The term “enantiomeric amino acids” refers to amino acids that can exist in at least two forms that are nonsuperimposable mirror images of each other. Most amino acids (except glycine) are enantiomeric and exist in a so-called L-form (L amino acid) or D-form (D amino acid). Most naturally occurring amino acids are “L” amino acids. The terms “D amino acid” and “L amino acid” are used to refer to absolute configuration of the amino acid, rather than a particular direction of rotation of plane-polarized light. The usage herein is consistent with standard usage by those of skill in the art. Amino acids are designated herein using standard 1-letter or three-letter codes, e.g. as designated in Standard ST.25 in the Handbook On Industrial Property Information and Documentation.

The term “protecting group” refers to a chemical group that, when attached to a functional group in an amino acid (e.g. a side chain, an alpha amino group, an alpha carboxyl group, etc.) blocks or masks the properties of that functional group. Preferred amino-terminal protecting groups include, but are not limited to acetyl, or amino groups. Other amino-terminal protecting groups include, but are not limited to alkyl chains as in fatty acids, propeonyl, formyl and others. Preferred carboxyl terminal protecting groups include, but are not limited to groups that form amides or esters.

The phrase “protect a phospholipid from oxidation by an oxidizing agent” refers to the ability of a compound to reduce the rate of oxidation of a phospholipid (or the amount of oxidized phospholipid produced) when that phospholipid is contacted with an oxidizing agent (e.g. hydrogen peroxide, 13-(S)-HPODE, 15-(S)-HPETE, HPODE, HPETE, HODE, HETE, etc.).

The terms “low density lipoprotein” or “LDL” is defined in accordance with common usage of those of skill in the art. Generally, LDL refers to the lipid-protein complex which when isolated by ultracentrifugation is found in the density range d=1.019 to d=1.063.

The terms “high density lipoprotein” or “HDL” is defined in accordance with common usage of those of skill in the art. Generally “HDL” refers to a lipid-protein complex which when isolated by ultracentrifugation is found in the density range of d=1.063 to d=1.21.

The term “Group I HDL” refers to a high density lipoprotein or components thereof (e.g. apo A-I, paraoxonase, platelet activating factor acetylhydrolase, etc.) that reduce oxidized lipids (e.g. in low density lipoproteins) or that protect oxidized lipids from oxidation by oxidizing agents.

The term “Group II HDL” refers to an HDL that offers reduced activity or no activity in protecting lipids from oxidation or in repairing (e.g. reducing) oxidized lipids.

The term “HDL component” refers to a component (e.g. molecules) that comprises a high density lipoprotein (HDL). Assays for HDL that protect lipids from oxidation or that repair (e.g. reduce oxidized lipids) also include assays for components of HDL (e.g. apo A-I, paraoxonase, platelet activating factor acetylhydrolase, etc.) that display such activity.

The term “human apo A-I peptide” refers to a full-length human apo A-I peptide or to a fragment or domain thereof comprising a class A amphipathic helix.

A “monocytic reaction” as used herein refers to monocyte activity characteristic of the “inflammatory response” associated with atherosclerotic plaque formation. The monocytic reaction is characterized by monocyte adhesion to cells of the vascular wall (e.g. cells of the vascular endothelium), and/or chemotaxis into the subendothelial space, and/or differentiation of monocytes into macrophages.

The term “absence of change” when referring to the amount of oxidized phospholipid refers to the lack of a detectable change, more preferably the lack of a statistically significant change (e.g. at least at the 85%, preferably at least at the 90%, more preferably at least at the 95%, and most preferably at least at the 98% or 99% confidence level). The absence of a detectable change can also refer to assays in which oxidized phospholipid level changes, but not as much as in the absence of the protein(s) described herein or with reference to other positive or negative controls.

The following abbreviations are used herein: PAPC: L-α-1-palmitoyl-2-arachidonoyl-sn-glycero-3-phosphocholine; POVPC: 1-palmitoyl-2-(5-oxovaleryl)-sn-glycero-3-phosphocholine; PGPC: 1-palmitoyl-2-glutaryl-sn-glycero-3-phosphocholine; PEIPC: 1-palmitoyl-2-(5,6-epoxyisoprostane E₂)-sn-glycero-3-phosphocholine; ChC18:2: cholesteryl linoleate; ChC18:2-OOH: cholesteryl linoleate hydroperoxide; DMPC: 1,2-ditetradecanoyl-rac-glycerol-3-phosphocholine; PON: paraoxonase; HPF: Standardized high power field; PAPC: L-α-1-palmitoyl-2-arachidonoyl-sn-glycero-3-phosphocholine; BL/6: C57BL/6J; C3H:C3H/HeJ.

The term “conservative substitution” is used in reference to proteins or peptides to reflect amino acid substitutions that do not substantially alter the activity (specificity (e.g. for lipoproteins))or binding affinity (e.g. for lipids or lipoproteins)) of the molecule. Typically conservative amino acid substitutions involve substitution one amino acid for another amino acid with similar chemical properties (e.g. charge or hydrophobicity). The following six groups each contain amino acids that are typical conservative substitutions for one another: 1) Alanine (A), Serine (S), Threonine (T); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); and 6) Phenylalanine (F), Tyrosine (Y), Tryptophan (W).

The terms “identical” or percent “identity,” in the context of two or more nucleic acids or polypeptide sequences, refer to two or more sequences or subsequences that are the same or have a specified percentage of amino acid residues or nucleotides that are the same, when compared and aligned for maximum correspondence, as measured using one of the following sequence comparison algorithms or by visual inspection. With respect to the peptides of this invention sequence identity is determined over the full length of the peptide.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman (1988) Proc. Natl. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of a useful algorithm is PILEUP. PILEUP creates a multiple sequence alignment from a group of related sequences using progressive, pairwise alignments to show relationship and percent sequence identity. It also plots a tree or dendogram showing the clustering relationships used to create the alignment. PILEUP uses a simplification of the progressive alignment method of Feng & Doolittle (1987) J. Mol. Evol. 35:351-360. The method used is similar to the method described by Higgins & Sharp (1989) CABIOS 5: 151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described in Altschul et al. (1990) J. Mol. Biol. 215: 403-410. Software for performing BLAST analyses is publicly available through the National Center for Biotechnology Information (http://www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=4, and a comparison of both strands. For amino acid sequences, the BLASTP program uses as defaults a wordlength (W) of 3, an expectation (E) of 10, and the BLOSUM62 scoring matrix (see Henikoff & Henikoff (1989) Proc. Natl. Acad. Sci. USA 89:10915).

In addition to calculating percent sequence identity, the BLAST algorithm also performs a statistical analysis of the similarity between two sequences (see, e.g., Karlin & Altschul (1993) Proc. Natl. Acad. Sci. USA, 90: 5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

### BRIEF DESCRIPTION OF THE DRAWINGS

FIG. 1 shows a comparison of the effect of D4F (Navab, et al. (2002) Circulation, 105: 290-292) and apoJ peptide 336 made from D amino acids (D-J336*) on the prevention of LDL-induced monocyte chemotactic activity in vitro in a co-incubation experiment. The data are mean±SD of the number of migrated monocytes in nine high power fields in quadruple cultures. (D-J336=Ac-LLEQLNEQFNWVSRLANLTQGE -NH₂, SEQ ID NO: 7).

FIG. 2 illustrates the prevention of LDL-induced monocyte chemotactic activity by pre-treatment of artery wall cells with D-J336 as compared to D-4F. The data are mean±SD of the number of migrated monocytes in nine high power fields in quadruple cultures.

FIG. 3 illustrates he effect of apo J peptide mimetics on HDL protective capacity in LDL receptor null mice. The values are the mean±SD of the number of migrated monocytes in 9 high power fields from each of quadruple assay wells.

FIG. 4 illustrates protection against LDL-induced monocyte chemotactic activity by HDL from apo E null mice given oral peptides. The values are the mean±SD of the number of migrated monocytes in 9 high power fields from each of quadruple assay wells. Asterisks indicate significant difference (p<0.05) as compared to No Peptide mHDL.

FIG. 5 illustrates the effect of oral apo A-1 peptide mimetic and apoJ peptide on LDL susceptibility to oxidation. The values are the mean±SD of the number of migrated monocytes in 9 high power fields from each of quadruple assay wells. Asterisks indicate significant difference (p<0.05) as compared to No Peptide LDL.

FIG. 6 illustrates the effect of oral apoA-1 peptide mimetic and apoJ peptide on HDL protective capacity. The values are the mean±SD of the number of migrated monocytes in 9 high power fields from each of quadruple assay wells. Asterisks indicate significant difference (p<0.05) as compared to No Peptide mHDL.

FIG. 7 illustrates the effect of oral apoA-1 peptide mimetic and apoJ peptide on plasma paraoxonase activity. The values are the mean±SD of readings from quadruple plasma aliquots. Asterisks indicate significant differences (p<0.05) as compared to No Peptide control plasma.

FIG. 8 shows the effect of oral G* peptides on HDL protective capacity in apoE−/−mice. The values are the mean±SD of readings from quadruple plasma aliquots. Asterisks indicate significant differences (p<0.05) as compared to no peptide control plasma.

FIG. 9 shows the effect of Oral G* peptide, 146-156, on HDL protective capacity in ApoE−/−mice.

FIGS. 10A through 10C illustrate helical wheel diagrams of certain peptides of this invention. FIG. 10A: V²W³A⁵F^{10,17}-D-4F; FIG. 10B: W³-D-4F; FIG. 10C: V²W³F¹⁰-D-4F:

FIG. 11 A standard human LDL (LDL) was added to human artery wall cocultures without (No Addition) or with human HDL (+Control HDL) or with mouse HDL from apoE null mice given Chow overnight (+Chow HDL), or given D-4F in the chow overnight (+D4F HDL) or given G5-D-4F in the chow overnight (+G5 HDL), or given G5,10-D-4F in the chow overnight (+5-10 HDL), or given G5,11-D-4F in the chow overnight (+5-11 HDL) and the resulting monocyte chemotactic activity determined as previously described (Navab M, Anantharamaiah, G M, Hama S, Garber D W, Chaddha M, Hough G, Lallone R, Fogelman A M. Oral administration of an apo A-I mimetic peptide synthesized from D-amino acids dramatically reduces atherosclerosis in mice independent of plasma cholesterol. Circulation 2002; 105:290-292.).

FIG. 12 shows that peptides of this invention are effective in mitigating symptoms of diabetes (e.g. blood glucose). Obese Zucker Rats 26 weeks of age were bled and then treated with daily intraperitoneal injections of D-4F (5.0 mg/kg/day). After 10 days the rats were bled again plasma glucose and lipid hydroperoxides (LOOH) were determined. *p=0.027; **p=0.0017.

FIG. 13 illustrates the effect of D4F on balloon injury of the carotid artery. Sixteen week old Obese Zucker Rats were injected with D-4F (5 mg/kg/daily) for 1 week at which time they underwent balloon injury of the common carotid artery. Two weeks later the rats were sacrificed and the intimal media ratio determined.

FIGS. 14A through 14K provide data demonstrating the purity of the various compounds produced in the solution phase chemistry.

FIG. 15 demonstrates that the product of the solution phase synthesis scheme is very biologically active in producing HDL and pre-beta HDL that inhibit LDL-induced monocyte chemotaxis in apo E null mice. ApoE null mice were fed 5 micrograms of the D-4F synthesized as described above (Frgmnt) or the mice were given the same amount of mouse chow without D-4F (Chow). Twelve hours after the feeding was started, the mice were bled and their plasma was fractionated on FPLC. LDL (100 micrograms LDL-cholesterol) was added to cocultures of human artery wall cells alone (LDL) or with a control human HDL (Control HDL) or with HDL (50 micrograms HDL-cholesterol) or post-HDL (pHDL; prebeta HDL) from mice that did (Frgmnt) or did not (Chow) receive the D-4F and the monocyte chemotactic activity produced was determined.

FIG. 16 illustrates the effect of various peptides of this invention on HDL paraoxonase activity.

FIG. 17 illustrates the effect of the of LAEYHAK (SEQ ID NO: 8) peptide on monocyte chemotactic activity. *p<0.001+hHDL versus hLDL; **p<0.001+Monkey HDL 6 hours after peptide versus+Monkey HDL Time Zero; ***p<0.001+Monkey LDL 6 hours after peptide versus+Monkey LDL Time Zero; ¶p<0.001+Monkey LDL Time Zero versus hLDL.

FIGS. 18A and 18B illustrate one embodiment of a stent according to the present invention. FIG. 18A schematically illustrates a drug-polymer stent 1800 comprises a stent framework 1820 with a plurality of reservoirs 1830 formed therein, and a drug polymer 1840 comprising one or more of the active agent(s) described herein (e.g., 4F, D4F, etc.) with an optional polymer layer positioned on the drug polymer. FIG. 18B schematically illustrates a vascular condition treatment system 1850 includes a stent framework 1870, a plurality of reservoirs 1890 formed in the stent framework, a drug polymer 1880 with a polymer layer, and a catheter 1040 coupled to stent framework 1880. Catheter 1860 may include a balloon used to expand the stent, or a sheath that retracts to allow expansion of the stent. Drug polymer 1880 includes one or more of the active agents described herein. The polymer layer can optionally comprise a barrier layer, a cap layer, or another drug polymer. The polymer layer typically provides a controlled drug-elution characteristic for each active agent. Drug elution refers to the transfer of the active agent(s) out from drug polymer 1880. The elution is determined as the total amount of bioactive agent excreted out of the drug polymer, typically measured in units of weight such as micrograms, or in weight per peripheral area of the stent.

### DETAILED DESCRIPTION

In certain embodiments this invention pertains to the identification of a number of active agents (e.g., peptides and/or certain small organic molecules) effective at mitigating a symptom of atherosclerosis or other conditions characterized by an inflammatory response. It is believed that administration of one active agent or two or more active agents in combination is effective to convert pro-inflammatory HDL to anti-inflammatory HDL, or to make anti-inflammatory HDL more anti-inflammatory. In certain embodiments such “conversion” is characterized by an increase in paraoxonase activity.

It was a surprising discovery that certain amphipathic helical peptides, e.g. class A and G* peptide described herein as well as other agents described herein possess anti-inflammatory properties and are capable of mediating a symptom of atherosclerosis or other pathology characterized by an inflammatory response (e.g., rheumatoid arthritis, lupus erythematous, polyarteritis nodosa, and osteoporosis).

In certain embodiments, the peptides are amphipathic helical peptides analogues possessing distributed charged residues (positively and/or negatively charged residues) on the polar face of the peptide and possessing a wide nonpolar face (termed a globular protein like, G*) amphipathic helical domain. Such amphipathic helical G* domains are characteristic of apo J and certain other apoproteins (e.g. apo M, apo AI, apo AIV, apo E, apo CII, apo CIII, and the like, but typically not apo A-II or apo C-I).

In certain embodiments the peptides of this invention comprise or consist of a class A amphipathic helix, and certain modified class A amphipathic helix peptides described herein have changes in the hydrophobic face of the molecule that improve activity and/or serum half-life.

In certain embodiments the peptides of this invention are small peptides that contain at least one dimethyltyrosine. Also provided are small peptides containing or comprising the amino acid sequence LAEYHAK (SEQ ID NO:8) comprising one or more protecting groups and/or one or more D residues. Certain small peptides comprise acidic or basic aminono acids alternating with aromatic or hydrophobic amino acids. Certain of the foregoing peptides exclude LAEYHAK (SEQ ID NO:8) comprising all L residues.

In various embodiments the peptides of this invention preferably range from about 6 or 10 amino acids to about 100 amino acids in length, more preferably from about 10 to about 60 or 80 amino acids in length, and most preferably from about 10, 15, or 20 amino acids to about 40 or 50 amino acids in length. In certain embodiments, the peptides range from about 6 or 10 to about 30 or 40 amino acids in length. Certain particularly preferred peptides of this invention show greater than about 40%, preferably greater than about 50% or 60%, more preferably greater than about 70% or 80% and most preferably greater than about 90% or 95% sequence identity with apo J or fragments thereof (ranging in length from about 10 to about 40 amino acids, e.g. over the same length as the peptide in question).

It was a surprising discovery of this invention that such peptides, particularly when comprising one or more D-form amino acids retain the biological activity of the corresponding L-form peptide. Moreover, these peptides show in vivo activity, even when delivered orally. The peptides show elevated serum half-life, and the ability to mitigate or prevent/inhibit one or more symptoms of atherosclerosis.

We discovered that normal HDL inhibits three steps in the formation of mildly oxidized LDL. In those studies (see, e.g. WO 02/15923) we demonstrated that treating human LDL in vitro with apo A-I or an apo A-I mimetic peptide (37pA) removed seeding molecules from the LDL that included HPODE and HPETE. These seeding molecules were required for cocultures of human artery wall cells to be able to oxidize LDL and for the LDL to induce the artery wall cells to produce monocyte chemotactic activity. We also demonstrated that after injection of apo A-I into mice or infusion into humans, the LDL isolated from the mice or human volunteers was resistant to oxidation by human artery wall cells and did not induce monocyte chemotactic activity in the artery wall cell cocultures.

Without being bound to a particular theory, we believe the active agents of this invention function in a manner similar to the activity of the apo A-I mimetics described in PCT publication WO 2002/15923. In particular, it is believed that the present invention functions in part by increasing the ant-inflammatory properties of HDL. In particular, we believe the peptides of this invention bind seeding molecules in LDL that are necessary for LDL oxidation and then carry the seeding molecules away where there are ultimately excreted.

We have demonstrated that oral administration of an apo AI mimetic peptide synthesized from D amino acids dramatically reduces atherosclerosis in mice independent of changes in plasma or HDL cholesterol concentrations. Similar to the action of the apo A-I mimetics, we believe that synthetic peptides mimicking the amphipathic helical domains of apo J that are synthesized from D amino acids, and other peptides described herein, can be given orally or by other routes including injection and will ameliorate atherosclerosis and other chronic inflammatory conditions.

In certain embodiments the peptides of this invention can comprise all L-form amino acids. However, peptides comprising one or more D-form amino acids and preferably all D-form amino acids (all enantiomeric amino acids are D form) provide for more effective delivery via oral administration and will be more stable in the circulation. Particularly preferred peptides are blocked at one or both termini (e.g., with the N-terminus acetylated and the C-terminus amidated).

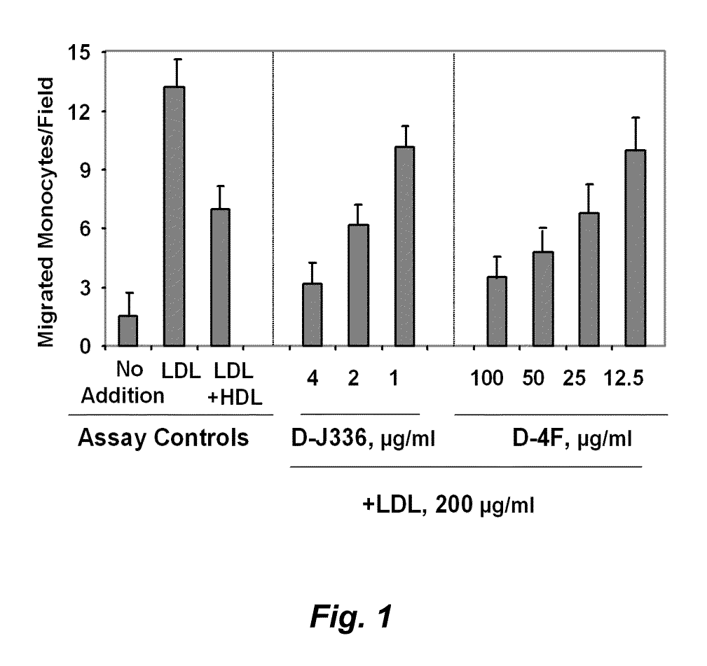

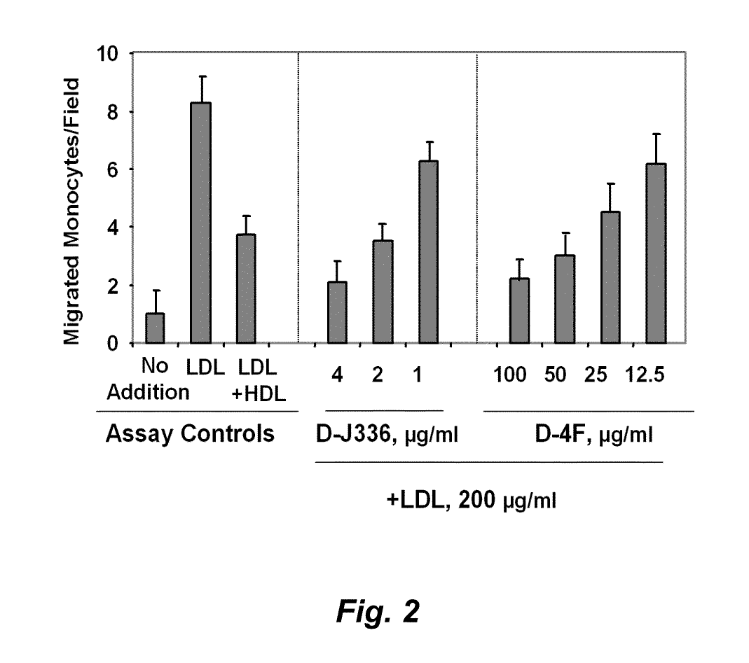

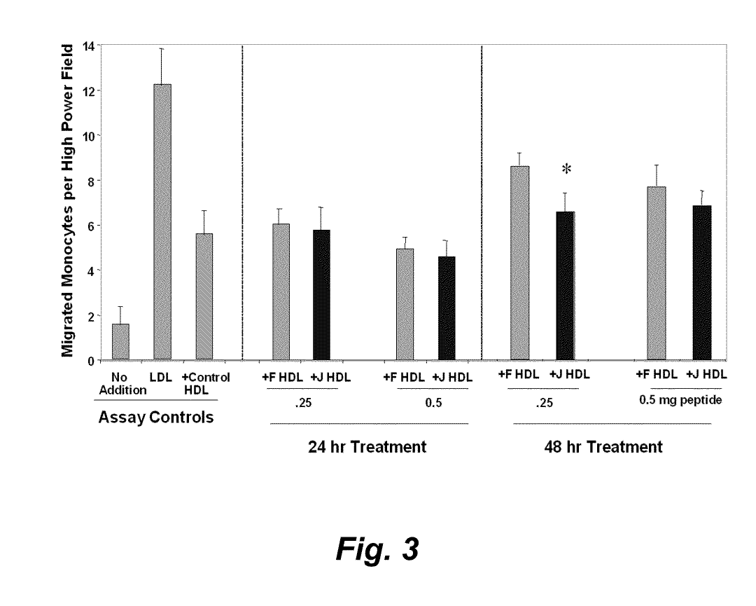

The protective function of the peptides of this invention is illustrated in Example 1. The in vitro concentration of the new class of peptides necessary to prevent LDL-induced monocyte chemotactic activity by human artery wall cells is 10 to 25 times less than the concentration required for an apoA-I mimetic (D4F) (compare DJ336 to D4F in FIG. 1). Similarly, in a preincubation the peptides of this invention were 10 to 25 times more potent in preventing LDL oxidation by artery wall cells (compare DJ336 to D4F in FIG. 2). As shown in FIG. 3, when DJ335 was given orally to LDL receptor null mice it was essentially as effective as D4F in rendering HDL more protective in preventing LDL-induced monocyte chemotactic activity.

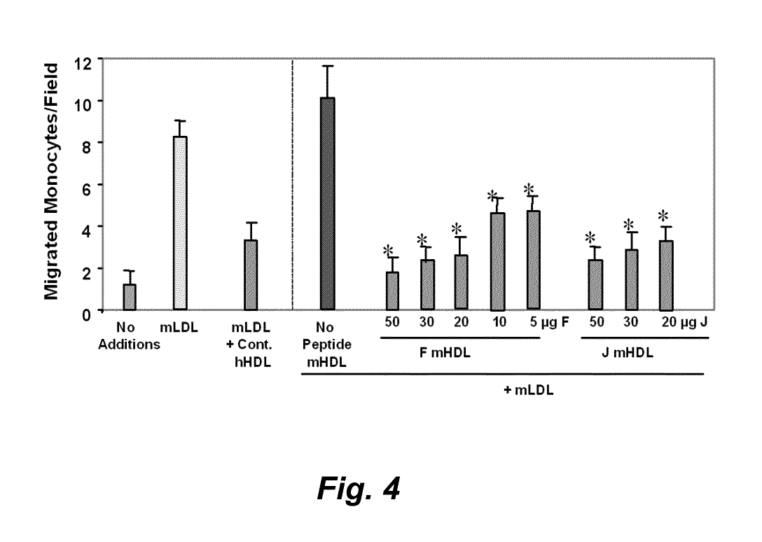

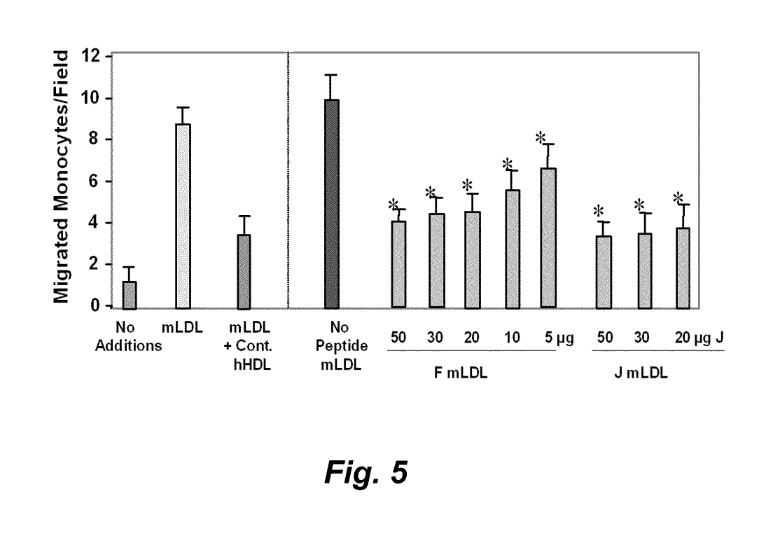

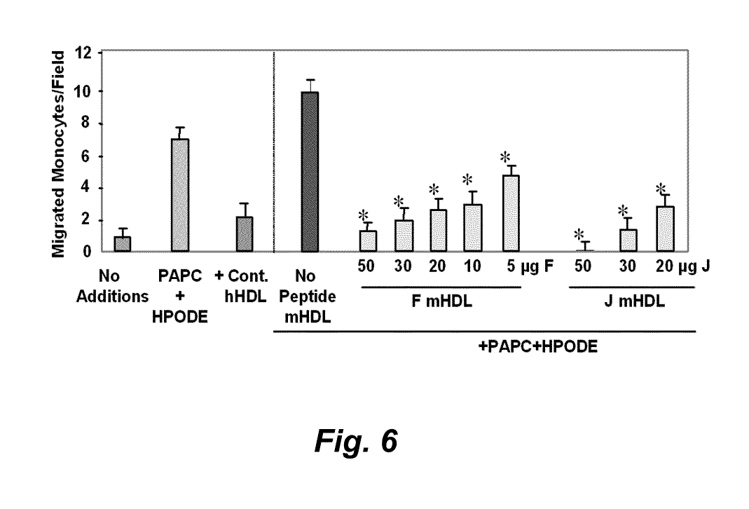

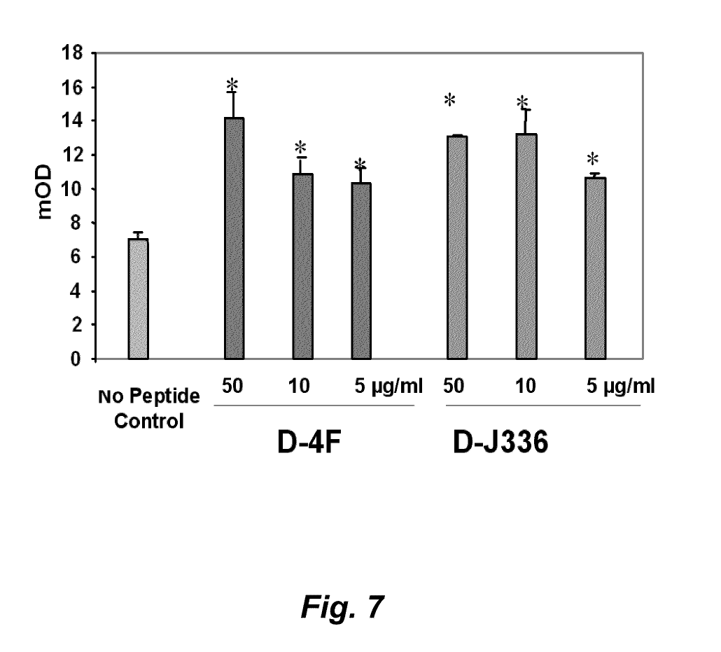

FIG. 4 demonstrates that when added to the drinking water a peptide of this invention (DJ336) was as potent as D4F in enhancing HDL protective capacity in apo E null mice. FIG. 5 demonstrates that, when added to the drinking water, a peptide of this invention DJ336 was slightly more potent than D4F in rendering the LDL from apo E null mice resistant to oxidation by human artery wall cells as determined by the induction of monocyte chemotactic activity. FIG. 6 demonstrates that when added to the drinking water DJ336 was as potent as D4F in causing HDL to inhibit the oxidation of a phospholipid PAPC by the oxidant HPODE in a human artery wall coculture as measured by the generation of monocyte chemotactic activity (see Navab et al. (2001) J. Lipid Res. 42: 1308-1317 for an explanation of the test system). FIG. 7 demonstrates that, when added to the drinking water, DJ336 was at least as potent as D4F in increasing the paraoxonase activity of apo E null mice.

In view of the foregoing, in one embodiment, this invention provides methods for ameliorating and/or preventing one or more symptoms of atherosclerosis and/or a pathology associated with (characterized by) an inflammatory response. The methods typically involve administering to an organism, preferably a mammal, more preferably a human one or more of the peptides, or other active agents, of this invention (or mimetics of such peptides). The agent(s) can be administered, as described herein, according to any of a number of standard methods including, but not limited to injection, suppository, nasal spray, time-release implant, transdermal patch, and the like. In one particularly preferred embodiment, the peptide(s) are administered orally (e.g. as a syrup, capsule, or tablet).

While the invention is described with respect to use in humans, it is also suitable for animal, e.g. veterinary use. Thus preferred organisms include, but are not limited to humans, non-human primates, canines, equines, felines, porcines, ungulates, largomorphs, and the like.

The methods of this invention are not limited to humans or non-human animals showing one or more symptom(s) of atherosclerosis (e.g. hypertension, plaque formation and rupture, reduction in clinical events such as heart attack, angina, or stroke, high levels of plasma cholesterol, high levels of low density lipoprotein, high levels of very low density lipoprotein, or inflammatory proteins, etc.), but are useful in a prophylactic context. Thus, the peptides of this invention (or mimetics thereof) may be administered to organisms to prevent the onset/development of one or more symptoms of atherosclerosis. Particularly preferred subjects in this context are subjects showing one or more risk factors for atherosclerosis (e.g. family history, hypertension, obesity, high alcohol consumption, smoking, high blood cholesterol, high blood triglycerides, elevated blood LDL, VLDL, IDL, or low HDL, diabetes, or a family history of diabetes, high blood lipids, heart attack, angina or stroke, etc.).

In addition to methods of use of the atherosclerosis-inhibiting peptides of this invention, this invention also provides the peptides themselves, the peptides formulated as pharmaceuticals, particularly for oral delivery, and kits for the treatment and/or prevention of one or more symptoms of atherosclerosis.

### I. Methods of Treatment.

The active agents (e.g. peptides, small organic molecules, amino acid pairs, etc.) described herein are effective for mitigating one or more symptoms and/or reducing the rate of onset and/or severity of one or more indications described herein. In particular, the active agents (e.g. peptides, small organic molecules, amino acid pairs, etc.) described herein are effective for mitigating one or more symptoms of atherosclerosis. Without being bound to a particular theory, it is believed that the peptides bind the “seeding molecules” required for the formation of pro-inflammatory oxidized phospholipids such as Ox-PAPC, POVPC, PGPC, and PEIPC.

In addition, since many inflammatory conditions and/or other pathologies are mediated at least in part by oxidized lipids, we believe that the peptides of this invention are effective in ameliorating conditions that are characterized by the formation of biologically active oxidized lipids. In addition, there are a number of other conditions for which the active agents described herein appear to be efficacious.

A number of pathologies for which the active agents described herein appear to be a palliative and/or a preventative are described below.

A) Atherosclerosis and Associated Pathologies.

We discovered that normal HDL inhibits three steps in the formation of mildly oxidized LDL. In particular, we demonstrated that treating human LDL in vitro with apo A-I or an apo A-I mimetic peptide (37 pA) removed seeding molecules from the LDL that included HPODE and HPETE. These seeding molecules were required for cocultures of human artery wall cells to be able to oxidize LDL and for the LDL to induce the artery wall cells to produce monocyte chemotactic activity. We also demonstrated that after injection of apo A-I into mice or infusion into humans, the LDL isolated from the mice or human volunteers after injection/infusion of apo A-I was resistant to oxidation by human artery wall cells and did not induce monocyte chemotactic activity in the artery wall cell cocultures.

The protective function of various active agents of this invention is illustrated in various related applications (see, e.g., PCT Publications WO 2002/15923, and WO 2004/034977, etc.). FIG. 1, panels A, B, C, and D in WO 2002/15923 show the association of ¹⁴C-D-5F with blood components in an ApoE null mouse. It is also demonstrated that HDL from mice that were fed an atherogenic diet and injected with PBS failed to inhibit the oxidation of human LDL and failed to inhibit LDL-induced monocyte chemotactic activity in human artery wall coculures. In contrast, HDL from mice fed an atherogenic diet and injected daily with peptides described herein was as effective in inhibiting human LDL oxidation and preventing LDL-induced monocyte chemotactic activity in the cocultures as was normal human HDL (FIGS. 2A and 2B in WO 02/15923). In addition, LDL taken from mice fed the atherogenic diet and injected daily with PBS was more readily oxidized and more readily induced monocyte chemotactic activity than LDL taken from mice fed the same diet but injected with 20 μg daily of peptide 5F. The D peptide did not appear to be immunogenic (FIG. 4 in WO 02/15923).

The in vitro responses of human artery wall cells to HDL and LDL from mice fed the atherogenic diet and injected with a peptide according to this invention are consistent with the protective action shown by such peptides in vivo. Despite, similar levels of total cholesterol, LDL-cholesterol, IDL+VLDL-cholesterol, and lower HDL-cholesterol as a percent of total cholesterol, the animals fed the atherogenic diet and injected with the peptide had significantly lower lesion scores (FIG. 5 in WO 02/15923). The peptides of this invention thus prevented progression of atherosclerotic lesions in mice fed an atherogenic diet.

Thus, in one embodiment, this invention provides methods for ameliorating and/or preventing one or more symptoms of atherosclerosis by administering one or more of the active agents described herein.

B) Mitigation of a Symptom or Condition Associated with Coronary Calcification and Osteoporosis.

Vascular calcification and osteoporosis often co-exist in the same subjects (Ouchi et al. (1993) Ann NY Acad Sci., 676: 297-307; Boukhris and Becker (1972) JAMA, 219: 1307-1311; Banks et al. (1994) Eur J Clin Invest., 24: 813-817; Laroche et al. (1994) Clin Rheumatol., 13: 611-614; Broulik and Kapitola (1993) Endocr Regul., 27: 57-60; Frye et al. (1992) Bone Mine., 19: 185-194; Barengolts et al. (1998) Calcif Tissue Int., 62: 209-213; Burnett and Vasikaran (2002) Ann Clin Biochem., 39: 203-210. Parhami et al. (1997) Arterioscl Thromb Vasc Biol., 17: 680-687, demonstrated that mildly oxidized LDL (MM-LDL) and the biologically active lipids in MM-LDL [i.e. oxidized 1-palmitoyl-2-arachidonoyl-sn-glycero-3-phosphorylcholine) (Ox-PAPC)], as well as the isoprostane, 8-iso prostaglandin E₂, but not the unoxidized phospholipid (PAPC) or isoprostane 8-iso progstaglandin F_{2α} induced alkaline phosphatase activity and osteoblastic differentiation of calcifying vascular cells (CVCs) in vitro, but inhibited the differentiation of MC3T3-E1 bone cells.

The osteon resembles the artery wall in that the osteon is centered on an endothelial cell-lined lumen surrounded by a subendothelial space containing matrix and fibroblast-like cells, which is in turn surrounded by preosteoblasts and osteoblasts occupying a position analogous to smooth muscle cells in the artery wall (Id.). Trabecular bone osteoblasts also interface with bone marrow subendothelial spaces (Id.). Parhami et al. postulated that lipoproteins could cross the endothelium of bone arteries and be deposited in the subendothelial space where they could undergo oxidation as in coronary arteries (Id.). Based on their in vitro data they predicted that LDL oxidation in the subendothelial space of bone arteries and in bone marrow would lead to reduced osteoblastic differentiation and mineralization which would contribute to osteoporosis (Id.). Their hypothesis further predicted that LDL levels would be positively correlated with osteoporosis as they are with coronary calcification (Pohle et al. (2001) Circulation, 104: 1927-1932), but HDL levels would be negatively correlated with osteoporosis (Parhami et al. (1997) Arterioscl Thromb Vasc Biol., 17: 680-687).

In vitro, the osteoblastic differentiation of the marrow stromal cell line M2-10B4 was inhibited by MM-LDL but not native LDL (Parhami et al. (1999) J Bone Miner Res., 14: 2067-2078). When marrow stromal cells from atherosclerosis susceptible C57BL/6 (BL6) mice fed a low fat chow diet were cultured there was robust osteogenic differentiation (Id.). In contrast, when the marrow stromal cells taken from the mice after a high fat, atherogenic diet were cultured they did not undergo osteogenic differentiation (Id.). This observation is particularly important since it provides a possible explanation for the decreased osteogenic potential of marrow stromal cells in the development of osteoporosis (Nuttall and Gimble (2000) Bone, 27: 177-184). In vivo the decrease in osteogenic potential is accompanied by an increase in adipogenesis in osteoporotic bone (Id.).

It was found that adding D-4F to the drinking water of apoE null mice for 6 weeks dramatically increased trabecular bone mineral density and it is believed that the other active agents of this invention will act similarly.

Our data indicate that osteoporosis can be regarded as an “atherosclerosis of bone”. It appears to be a result of the action of oxidized lipids. HDL destroys these oxidized lipids and promotes osteoblastic differentiation. Our data indicate that administering active agent(s) of this invention to a mammal (e.g., in the drinking water of apoE null mice) dramatically increases trabecular bone in just a matter of weeks.

This indicates that the active agents, described herein are useful for mitigation one or more symptoms of osteoporosis (e.g., for inhibiting decalcification) or for inducing recalcification of osteoporotic bone. The active agents are also useful as prophylactics to prevent the onset of symptom(s) of osteoporosis in a mammal (e.g., a patient at risk for osteoporosis).

We believe similar mechanisms are a cause of coronary calcification, e.g., calcific aortic stenosis. Thus, in certain embodiments, this invention contemplates the use of the active agents described herein to inhibit or prevent a symptom of a disease such as coronary calcification, calcific aortic stenosis, osteoporosis, and the like.

C) Inflammatory and Autoimmune Indications.

Chronic inflammatory and/or autoimmune conditions are also characterized by the formation of a number of reactive oxygen species and are amenable to treatment using one or more of the active agents described herein. Thus, without being bound to a particular theory, we also believe the active agents described herein are useful, prophylactically or therapeutically, to mitigate the onset and/or more or more symptoms of a variety of other conditions including, but not limited to rheumatoid arthritis, lupus erythematous, polyarteritis nodosa, polymyalgia rheumatica, lupus erythematosus, multiple sclerosis, and the like.

In certain embodiments, the active agents are useful in mitigating one or more symptoms caused by or associated with an inflammatory response in these conditions.

Also, In certain embodiments , the active agents are useful in mitigating one or more symptoms caused by or associated with an inflammatory response associated with AIDS.

D) Infections/Trauma/Transplants.

We have observed that a a consequence of influenza infection and other infenctions is the diminution in paraoxonase and platelet activating acetylhydrolase activity in the HDL. Without being bound by a particular theory, we believe that, as a result of the loss of these HDL enzymatic activities and also as a result of the association of pro-oxidant proteins with HDL during the acute phase response, HDL is no longer able to prevent LDL oxidation and is no longer able to prevent the LDL-induced production of monocyte chemotactic activity by endothelial cells.

We observed that in a subject injected with very low dosages of certain agents of this invention (e.g., 20 micrograms for mice) daily after infection with the influenza A virus paraoxonase levels did not fall and the biologically active oxidized phospholipids were not generated beyond background. This indicates that 4F, D4F (and/or other agents of this invention) can be administered (e.g. orally or by injection) to patients (including, for example with known coronary artery disease during influenza infection or other events that can generate an acute phase inflammatory response, e.g. due to viral infection, bacterial infection, trauma, transplant, various autoimmune conditions, etc.) and thus we can prevent by this short term treatment the increased incidence of heart attack and stroke associated with pathologies that generate such inflammatory states.

In addition, by restoring and/or maintaining paroxonase levels and/or monocyte activity, the agent(s) of this invention are useful in the treatment of infection (e.g., viral infection, bacterial infection, fungal infection) and/or the inflammatory pathologies associated with infection (e.g. meningitis), and/or trauma.

In certain embodiments, because of the combined anti-inflammatory activity and anti-infective activity, the agents described herein are also useful in the treatment of a wound or other trauma, mitigating adverse effects associated with organ or tissue transplant, and/or organ or tissue transplant rejection, and/or implanted prostheses, and/or transplant atherosclerosis, and/or biofilm formation. In addition, we believe that L-4F, D-4F, and/or other agents described herein are also useful in mitigating the effects of spinal cord injuries.

E) Diabetes and Associated Conditions.

Various active agents described herein have also been observed to show efficacy in reducing and/or preventing one or more symptoms associated with diabetes. Thus, in various embodiments, this invention provides methods of treating (therapeutically and/or prophylactically) diabetes and/or associated pathologies (e.g., type i diabetes, type ii diabetes, juvenile onset diabetes, diabetic nephropathy, nephropathy, diabetic neuropathy, diabetic retinopathy, and the like.

F) Inhibition of Restenosis.

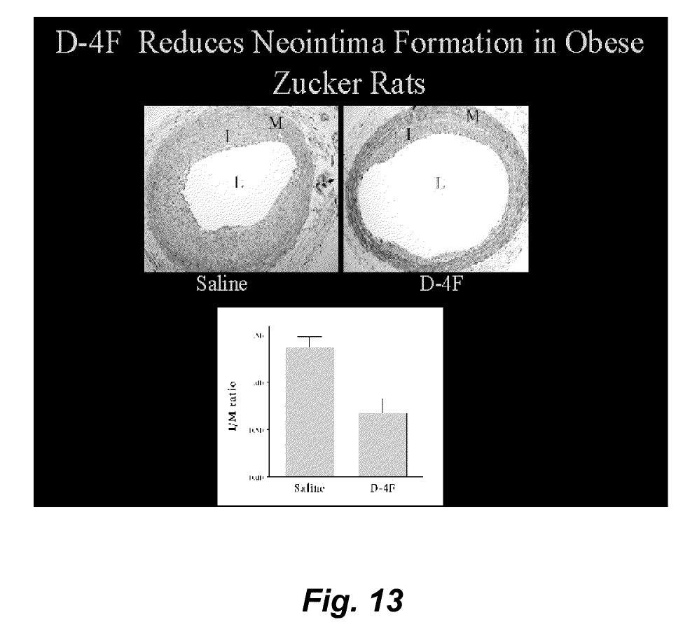

It is also demonstrated herein that the active agents of the present invention are effective for inhibiting restenosis, following, e.g., balloon angioplasty. Thus, for example, FIG. 13 shows the effect of the class A amphiphathic helical peptide D4F on balloon injury of the carotid artery. Sixteen week old Obese Zucker Rats were injected with D-4F (5 mg/kg/daily) for 1 week at which time they underwent balloon injury of the common carotid artery. Two weeks later the rats were sacrificed and the intimal media ratio determined. As shown in FIG. 13, restenoiss is reduced in the treated animals.

Thus, in certain embodiments, this invention contemplate administration of one or more active agents described herein to reduce/prevent restenosis. The agents can b e administered systemically (e.g., orally, by injection, and the like) or they can be delivered locally, e.g, by the use of drug-eluting stents and/or simply by local administration during the angioplasty procedure.

G) Mitigation of a Symptom of Atherosclerosis Associated with an Acute Inflammatory Response.

The active agents, of this invention are also useful in a number of contexts. For example, we have observed that cardiovascular complications (e.g., atherosclerosis, stroke, etc.) frequently accompany or follow the onset of an acute phase inflammatory response, e.g., such as that associated with a recurrent inflammatory disease, a viral infection (e.g., influenza), a bacterial infection, a fungal infection, an organ transplant, a wound or other trauma, and so forth.

Thus, in certain embodiments, this invention contemplates administering one or more of the active agents described herein to a subject at risk for, or incurring, an acute inflammatory response and/or at risk for or incurring a symptom of atherosclerosis and/or an associated pathology (e.g., stroke).

Thus, for example, a person having or at risk for coronary disease may prophylactically be administered a one or more active agents of this invention during flu season. A person (or animal) subject to a recurrent inflammatory condition, e.g., rheumatoid arthritis, various autoimmune diseases, etc., can be treated with a one or more agents described herein to mitigate or prevent the development of atherosclerosis or stroke. A person (or animal) subject to trauma, e.g., acute injury, tissue transplant, etc. can be treated with a polypeptide of this invention to mitigate the development of atherosclerosis or stroke.

In certain instances such methods will entail a diagnosis of the occurrence or risk of an acute inflammatory response. The acute inflammatory response typically involves alterations in metabolism and gene regulation in the liver. It is a dynamic homeostatic process that involves all of the major systems of the body, in addition to the immune, cardiovascular and central nervous system. Normally, the acute phase response lasts only a few days; however, in cases of chronic or recurring inflammation, an aberrant continuation of some aspects of the acute phase response may contribute to the underlying tissue damage that accompanies the disease, and may also lead to further complications, for example cardiovascular diseases or protein deposition diseases such as amyloidosis.

An important aspect of the acute phase response is the radically altered biosynthetic profile of the liver. Under normal circumstances, the liver synthesizes a characteristic range of plasma proteins at steady state concentrations. Many of these proteins have important functions and higher plasma levels of these acute phase reactants (APRs) or acute phase proteins (APPs) are required during the acute phase response following an inflammatory stimulus. Although most APRs are synthesized by hepatocytes, some are produced by other cell types, including monocytes, endothelial cells, fibroblasts and adipocytes. Most APRs are induced between 50% and several-fold over normal levels. In contrast, the major APRs can increase to 1000-fold over normal levels. This group includes serum amyloid A (SAA) and either C-reactive protein (CRP) in humans or its homologue in mice, serum amyloid P component (SAP). So-called negative APRs are decreased in plasma concentration during the acute phase response to allow an increase in the capacity of the liver to synthesize the induced APRs.

In certain embodiments, the acute phase response, or risk therefore is evaluated by measuring one or more APPs. Measuring such markers is well known to those of skill in the art, and commercial companies exist that provide such measurement (e.g., AGP measured by Cardiotech Services, Louisville, Ky.).

### II. Active Agents.

A wide variety of active agents are suitable for the treatment of one or more of the indications discussed herein. These agents include, but are not limited to class A amphipathic helical peptides, class A amphipathic helical peptide mimetics of apoA-I having aromatic or aliphatic residues in the non-polar face, small peptides including penta-peptides, tetrapeptides, tripeptides, dipeptides and pairs of amino acids, Apo-J (G* peptides), and peptide mimetics, e.g., as described below.

A) Class A Amphipathic Helical Peptides.

In certain embodiments, the activate agents for use in the method of this invention include class A amphipathic helical peptides, e.g. as described in U.S. Pat. No. 6,664,230, and PCT Publications WO 02/15923 and WO 2004/034977. It was discovered that peptides comprising a class A amphipathic helix (“class A peptides”), in addition to being capable of mitigating one or more symptoms of atherosclerosis are also useful in the treatment of one or more of the other indications described herein.

Class A peptides are characterized by formation of an α-helix that produces a segregation of polar and non-polar residues thereby forming a polar and a nonpolar face with the positively charged residues residing at the polar-nonpolar interface and the negatively charged residues residing at the center of the polar face (see, e.g., Anantharamaiah (1986) Meth. Enzymol, 128: 626-668). It is noted that the fourth exon of apo A-I, when folded into 3.667 residues/turn produces a class A amphipathic helical structure.

One class A peptide designated 18A (see, e.g., Anantharamaiah (1986) Meth. Enzymol, 128: 626-668) was modified as described herein to produce peptides orally administratable and highly effective at inhibiting or preventing one or more symptoms of atherosclerosis and/or other indications described herein. Without being bound by a particular theory, it is believed that the peptides of this invention may act in vivo may by picking up seeding molecule(s) that mitigate oxidation of LDL.

We determined that increasing the number of Phe residues on the hydrophobic face of 18A would theoretically increase lipid affinity as determined by the computation described by Palgunachari et al. (1996) Arteriosclerosis, Thrombosis, & Vascular Biology 16: 328-338. Theoretically, a systematic substitution of residues in the nonpolar face of 18A with Phe could yield six peptides. Peptides with an additional 2, 3 and 4 Phe would have theoretical lipid affinity (λ) values of 13, 14 and 15 units, respectively. However, the λ values jumped four units if the additional Phe were increased from 4 to 5 (to 19 λ units). Increasing to 6 or 7 Phe would produce a less dramatic increase (to 20 and 21 λ units, respectively).

A number of these class A peptides were made including, the peptide designated 4F, D4F, 5F, and D5F, and the like. Various class A peptides inhibited lesion development in atherosclerosis-susceptible mice. In addition, the peptides show varying, but significant degrees of efficacy in mitigating one or more symptoms of the various pathologies described herein. A number of such peptides are illustrated in Table 1.

TABLE 1
Illustrative class A amphipathic helical
peptides for use in this invention.

| | | SEQ |
|---|---|---|
| Peptide | | ID |
| Name | Amino Acid Sequence | NO. |
| 18A | D-W-L-K-A-F-Y-D-K-V-A-E-K-L-K-E-A-F | 9 |
| 2F | Ac-D-W-L-K-A-F-Y-D-K-V-A-E-K-L-K-E-A-F-NH₂ | 10 |
| 3F | Ac-D-W-F-K-A-F-Y-D-K-V-A-E-K-L-K-E-A-F-NH₂ | 11 |
| 3F14 | Ac-D-W-L-K-A-F-Y-D-K-V-A-E-K-F-K-E-A-F-NH₂ | 12 |
| 4F | Ac-D-W-F-K-A-F-Y-D-K-V-A-E-K-F-K-E-A-F-NH₂ | 13 |
| 5F | Ac-D-W-L-K-A-F-Y-D-K-V-F-E-K-F-K-E-F-F-NH₂ | 14 |
| 6F | Ac-D-W-L-K-A-F-Y-D-K-F-F-E-K-F-K-E-F-F-NH₂ | 15 |
| 7F | Ac-D-W-F-K-A-F-Y-D-K-F-F-E-K-F-K-E-F-F-NH₂ | 16 |
| | Ac-D-W-L-K-A-F-Y-D-K-V-A-E-K-L-K-E-F-F-NH₂ | 17 |
| | Ac-D-W-L-K-A-F-Y-D-K-V-F-E-K-F-K-E-A-F-NH₂ | 18 |
| | Ac-D-W-L-K-A-F-Y-D-K-V-F-E-K-L-K-E-F-F-NH₂ | 19 |
| | Ac-D-W-L-K-A-F-Y-D-K-V-A-E-K-F-K-E-F-F-NH₂ | 20 |
| | Ac-D-W-L-K-A-F-Y-D-K-V-F-E-K-F-K-E-F-F-NH₂ | 21 |
| | Ac-E-W-L-K-L-F-Y-E-K-V-L-E-K-F-K-E-A-F-NH₂ | 22 |
| | Ac-E-W-L-K-A-F-Y-D-K-V-A-E-K-F-K-E-A-F-NH₂ | 23 |
| | Ac-E-W-L-K-A-F-Y-D-K-V-A-E-K-L-K-E-F-F-NH₂ | 24 |
| | Ac-E-W-L-K-A-F-Y-D-K-V-F-E-K-F-K-E-A-F-NH₂ | 25 |
| | Ac-E-W-L-K-A-F-Y-D-K-V-F-E-K-L-K-E-F-F-NH₂ | 26 |
| | Ac-E-W-L-K-A-F-Y-D-K-V-A-E-K-F-K-E-F-F-NH₂ | 27 |
| | Ac-E-W-L-K-A-F-Y-D-K-V-F-E-K-F-K-E-F-F-NH₂ | 28 |
| | AC-A-F-Y-D-K-V-A-E-K-L-K-E-A-F-NH₂ | 29 |
| | Ac-A-F-Y-D-K-V-A-E-K-F-K-E-A-F-NH₂ | 30 |
| | Ac-A-F-Y-D-K-V-A-E-K-F-K-E-A-F-NH₂ | 31 |
| | Ac-A-F-Y-D-K-F-F-E-K-F-K-E-F-F-NH₂ | 32 |
| | Ac-A-F-Y-D-K-F-F-E-K-F-K-E-F-F-NH₂ | 33 |
| | Ac-A-F-Y-D-K-V-A-E-K-F-K-E-A-F-NH₂ | 34 |
| | Ac-A-F-Y-D-K-V-A-E-K-L-K-E-F-F-NH₂ | 35 |
| | Ac-A-F-Y-D-K-V-F-E-K-F-K-E-A-F-NH₂ | 36 |
| | Ac-A-F-Y-D-K-V-F-E-K-L-K-E-F-F-NH₂ | 37 |
| | Ac-A-F-Y-D-K-V-A-E-K-F-K-E-F-F-NH₂ | 38 |
| | Ac-K-A-F-Y-D-K-V-F-E-K-F-K-E-F-NH₂ | 39 |
| | Ac-L-F-Y-E-K-V-L-E-K-F-K-E-A-F-NH₂ | 40 |
| | Ac-A-F-Y-D-K-V-A-E-K-F-K-E-A-F-NH₂ | 41 |
| | Ac-A-F-Y-D-K-V-A-E-K-L-K-E-F-F-NH₂ | 42 |
| | Ac-A-F-Y-D-K-V-F-E-K-F-K-E-A-F-NH₂ | 43 |
| | Ac-A-F-Y-D-K-V-F-E-K-L-K-E-F-F-NH₂ | 44 |
| | Ac-A-F-Y-D-K-V-A-E-K-F-K-E-F-F-NH₂ | 45 |
| | Ac-A-F-Y-D-K-V-F-E-K-F-K-E-F-F-NH₂ | 46 |
| | Ac-D-W-L-K-A-L-Y-D-K-V-A-E-K-L-K-E-A-L-NH₂ | 47 |
| | Ac-D-W-F-K-A-F-Y-E-K-V-A-E-K-L-K-E-F-F-NH₂ | 48 |
| | Ac-D-W-F-K-A-F-Y-E-K-F-F-E-K-F-K-E-F-F-NH₂ | 49 |
| | Ac-E-W-L-K-A-L-Y-E-K-V-A-E-K-L-K-E-A-L-NH₂ | 50 |
| | Ac-E-W-L-K-A-F-Y-E-K-V-A-E-K-L-K-E-A-F-NH₂ | 51 |
| | Ac-E-W-F-K-A-F-Y-E-K-V-A-E-K-L-K-E-F-F-NH₂ | 52 |
| | Ac-E-W-L-K-A-F-Y-E-K-V-F-E-K-F-K-E-F-F-NH₂ | 53 |
| | Ac-E-W-L-K-A-F-Y-E-K-F-F-E-K-F-K-E-F-F-NH₂ | 54 |
| | Ac-E-W-F-K-A-F-Y-E-K-F-F-E-K-F-K-E-F-F-NH₂ | 55 |
| | Ac-D-F-L-K-A-W-Y-D-K-V-A-E-K-L-K-E-A-W-NH₂ | 56 |
| | Ac-E-F-L-K-A-W-Y-E-K-V-A-E-K-L-K-E-A-W-NH₂ | 57 |
| | Ac-D-F-W-K-A-W-Y-D-K-V-A-E-K-L-K-E-W-W-NH₂ | 58 |
| | Ac-E-F-W-K-A-W-Y-E-K-V-A-E-K-L-K-E-W-W-NH₂ | 59 |
| | Ac-D-K-L-K-A-F-Y-D-K-V-F-E-W-A-K-E-A-F-NH₂ | 60 |
| | Ac-D-K-W-K-A-V-Y-D-K-F-A-E-A-F-K-E-F-L-NH₂ | 61 |
| | Ac-E-K-L-K-A-F-Y-E-K-V-F-E-W-A-K-E-A-F-NH₂ | 62 |
| | Ac-E-K-W-K-A-V-Y-E-K-F-A-E-A-F-K-E-F-L-NH₂ | 63 |
| | Ac-D-W-L-K-A-F-V-D-K-F-A-E-K-F-K-E-A-Y-NH₂ | 64 |
| | Ac-E-K-W-K-A-V-Y-E-K-F-A-E-A-F-K-E-F-L-NH₂ | 65 |
| | Ac-D-W-L-K-A-F-V-Y-D-K-V-F-K-L-K-E-F-F-NH₂ | 66 |
| | Ac-E-W-L-K-A-F-V-Y-E-K-V-F-K-L-K-E-F-F-NH₂ | 67 |
| | Ac-D-W-L-R-A-F-Y-D-K-V-A-E-K-L-K-E-A-F-NH₂ | 68 |
| | Ac-E-W-L-R-A-F-Y-E-K-V-A-E-K-L-K-E-A-F-NH₂ | 69 |
| | Ac-D-W-L-K-A-F-Y-D-R-V-A-E-K-L-K-E-A-F-NH₂ | 70 |
| | Ac-E-W-L-K-A-F-Y-E-R-V-A-E-K-L-K-E-A-F-NH₂ | 71 |
| | Ac-D-W-L-K-A-F-Y-D-K-V-A-E-R-L-K-E-A-F-NH₂ | 72 |
| | Ac-E-W-L-K-A-F-Y-E-K-V-A-E-R-L-K-E-A-F-NH₂ | 73 |
| | Ac-D-W-L-K-A-F-Y-D-K-V-A-E-K-L-R-E-A-F-NH₂ | 74 |
| | Ac-E-W-L-K-A-F-Y-E-K-V-A-E-K-L-R-E-A-F-NH₂ | 75 |
| | Ac-D-W-L-K-A-F-Y-D-R-V-A-E-R-L-K-E-A-F-NH₂ | 76 |
| | Ac-E-W-L-K-A-F-Y-E-R-V-A-E-R-L-K-E-A-F-NH₂ | 77 |
| | Ac-D-W-L-R-A-F-Y-D-K-V-A-E-K-L-R-E-A-F-NH₂ | 78 |
| | Ac-E-W-L-R-A-F-Y-E-K-V-A-E-K-L-R-E-A-F-NH₂ | 79 |
| | Ac-D-W-L-R-A-F-Y-D-R-V-A-E-K-L-K-E-A-F-NH₂ | 80 |
| | Ac-E-W-L-R-A-F-Y-E-R-V-A-E-K-L-K-E-A-F-NH₂ | 81 |
| | Ac-D-W-L-K-A-F-Y-D-K-V-A-E-R-L-R-E-A-F-NH₂ | 82 |
| | Ac-E-W-L-K-A-F-Y-E-K-V-A-E-R-L-R-E-A-F-NH₂ | 83 |
| | Ac-D-W-L-R-A-F-Y-D-K-V-A-E-R-L-K-E-A-F-NH₂ | 84 |
| | Ac-E-W-L-R-A-F-Y-E-K-V-A-E-R-L-K-E-A-F-NH₂ | 85 |
| | D-W-L-K-A-F-Y-D-K-V-A-E-K-L-K-E-A-F-P-D- | 86 |
| | W-L-K-A-F-Y-D-K-V-A-E-K-L-K-E-A-F | |
| | D-W-L-K-A-F-Y-D-K-V-A-E-K-L-K-E-F-F-P-D- | 87 |
| | W-L-K-A-F-Y-D-K-V-A-E-K-L-K-E-F-F | |
| | D-W-F-K-A-F-Y-D-K-V-A-E-K-L-K-E-A-F-P-D- | 88 |
| | W-F-K-A-F-Y-D-K-V-A-E-K-L-K-E-A-F | |
| | D-K-L-K-A-F-Y-D-K-V-F-E-W-A-K-E-A-F-P-D- | 89 |
| | K-L-K-A-F-Y-D-K-V-F-E-W-L-K-E-A-F | |
| | D-K-W-K-A-V-Y-D-K-F-A-E-A-F-K-E-F-L-P-D- | 90 |
| | K-W-K-A-V-Y-D-K-F-A-E-A-F-K-E-F-L | |
| | D-W-F-K-A-F-Y-D-K-V-A-E-K-F-K-E-A-F-P-D- | 91 |
| | W-F-K-A-F-Y-D-K-V-A-E-K-F-K-E-A-F | |
| | D-W-L-K-A-F-V-Y-D-K-V-F-K-L-K-E-F-F-P-D- | 92 |
| | W-L-K-A-F-V-Y-D-K-V-F-K-L-K-E-F-F | |
| | D-W-L-K-A-F-Y-D-K-F-A-E-K-F-K-E-F-F-P-D- | 93 |
| | W-L-K-A-F-Y-D-K-F-A-E-K-F-K-E-F-F | |
| | Ac-E-W-F-K-A-F-Y-E-K-V-A-E-K-F-K-E-A-F- | 94 |
| | NH₂ | |
| | Ac-D-W-F-K-A-F-Y-D-K-V-A-E-K-F-NH₂ | 95 |
| | Ac-F-K-A-F-Y-D-K-V-A-E-K-F-K-E-NH₂ | 96 |
| | Ac-F-K-A-F-Y-E-K-V-A-E-K-F-K-E-NH₂ | 97 |
| | NMA-F-K-A-F-Y-D-K-V-A-E-K-F-K-E-NH₂ | 98 |
| | NMA-F-K-A-F-Y-E-K-V-A-E-K-F-K-E-NH₂ | 99 |
| | NMA-D-W-F-K-A-F-Y-D-K-V-A-E-K-F-K-E-A-F- | 100 |
| | NH₂ | |
| | NMA-E-W-F-K-A-F-Y-E-K-V-A-E-K-F-K-E-A-F- | 101 |
| | NH₂ | |
| | NMA-A-F-Y-D-K-V-A-E-K-F-K-E-A-F-NH2 | 102 |
| | NMA-D-W-F-K-A-F-Y-D-K-V-A-E-K-F-NH2 | 103 |
| | Ac-D-W-L-K-A-F-Y-D-K-V-F-E-K-F-K-E-F-F- | 104 |
| | NH₂ | |
| | NMA-D-W-L-K-A-F-Y-D-K-V-F-E-K-F-K-E-F-F- | |
| | NH₂ | |
| | Ac-E-W-L-K-A-F-Y-E-K-V-F-E-K-F-K-E-F-F- | 105 |
| | NH₂ | |
| | NMA-E-W-L-K-A-F-Y-E-K-V-F-E-K-F-K-E-F-F- | |
| | NH₂ | |
| | Ac-A-F-Y-D-K-V-F-E-K-F-K-E-F-F-NH₂ | 106 |
| | NMA-A-F-Y-D-K-V-F-E-K-F-K-E-F-F-NH₂ | |
| | Ac-A-F-Y-E-K-V-F-E-K-F-K-E-F-F-NH₂ | 107 |
| | NMA-A-F-Y-E-K-V-F-E-K-F-K-E-F-F-NH₂ | |
| | Ac-D-W-L-K-A-F-Y-D-K-V-F-E-K-F-NH₂ | 108 |
| | NMA-D-W-L-K-A-F-Y-D-K-V-F-E-K-F-NH₂ | |
| | Ac-E-W-L-K-A-F-Y-E-K-V-F-E-K-F-NH₂ | 109 |
| | NMA-E-W-L-K-A-F-Y-E-K-V-F-E-K-F-NH₂ | |
| | Ac-L-K-A-F-Y-D-K-V-F-E-K-F-K-E-NH₂ | 110 |
| | NMA-L-K-A-F-Y-D-K-V-F-E-K-F-K-E-NH₂ | |
| | Ac-L-K-A-F-Y-E-K-V-F-E-K-F-K-E-NH₂ | 111 |
| | NMA-L-K-A-F-Y-E-K-V-F-E-K-F-K-E-NH₂ | |

¹Linkers are underlined.
NMA is N-Methyl Anthranilyl.

In certain preferred embodiments, the peptides include variations of 4F (SEQ ID NO:13 in Table 1), also known as L-4F, where all residues are L form amino acids) or D-4F where one or more residues are D form amino acids). In any of the peptides described herein, the C-terminus, and/or N-terminus, and/or internal residues can be blocked with one or more blocking groups as described herein.

While various peptides of Table 1, are illustrated with an acetyl group or an N-methylanthranilyl group protecting the amino terminus and an amide group protecting the carboxyl terminus, any of these protecting groups may be eliminated and/or substituted with another protecting group as described herein. In particularly preferred embodiments, the peptides comprise one or more D-form amino acids as described herein. In certain embodiments, every amino acid (e.g., every enantiomeric amino acid) of the peptides of Table 1 is a D-form amino acid.

It is also noted that Table 1 is not fully inclusive. Using the teachings provided herein, other suitable class A amphipathic helical peptides can routinely be produced (e.g., by conservative or semi-conservative substitutions (e.g., D replaced by E), extensions, deletions, and the like). Thus, for example, one embodiment utilizes truncations of any one or more of peptides shown herein (e.g., peptides identified by SEQ ID Nos:10-28 and 47- in Table 1). Thus, for example, SEQ ID NO:29 illustrates a peptide comprising 14 amino acids from the C-terminus of 18A comprising one or more D amino acids, while SEQ ID NOS:30-46 illustrate other truncations.

Longer peptides are also suitable. Such longer peptides may entirely form a class A amphipathic helix, or the class A amphipathic helix (helices) can form one or more domains of the peptide. In addition, this invention contemplates multimeric versions of the peptides (e.g., concatamers). Thus, for example, the peptides illustrated herein can be coupled together (directly or through a linker (e.g., a carbon linker, or one or more amino acids) with one or more intervening amino acids). Illustrative polymeric peptides include 18A-Pro-18A and the peptides of SEQ ID NOs:86-93, in certain embodiments comprising one or more D amino acids, more preferably with every amino acid a D amino acid as described herein and/or having one or both termini protected.

B) Other Class A Amphipathic Helical Peptide Mimetics of apoA-I Having Aromatic or Aliphatic Residues in the Non-Polar Face.

In certain embodiments, this invention also provides modified class A amphipathic helix peptides. Certain preferred peptides incorporate one or more aromatic residues at the center of the nonpolar face, e.g., 3F^{cπ}, (as present in 4F), or with one or more aliphatic residues at the center of the nonpolar face, e.g., 3F^{Iπ}, see, e.g., Table 2. Without being bound to a particular theory, we believe the central aromatic residues on the nonpolar face of the peptide 3F^{cm}, due to the presence of TC electrons at the center of the nonpolar face, allow water molecules to penetrate near the hydrophobic lipid alkyl chains of the peptide-lipid complex, which in turn would enable the entry of reactive oxygen species (such as lipid hydroperoxides) shielding them from the cell surface. Similarly, we also believe the peptides with aliphatic residues at the center of the nonpolar face, e.g., 3F^{Iπ}, will act similarly but not quite as effectively as 3F^{Cπ}.

Preferred peptides will convert pro-inflammatory HDL to anti-inflammatory HDL or make anti-inflammatory HDL more anti-inflammatory, and/or decrease LDL-induced monocyte chemotactic activity generated by artery wall cells equal to or greater than D4F or other peptides shown in Table 1.

TABLE 2
Examples of certain preferred peptides.

| Name | Sequence | SEQ ID NO |
|---|---|---|
| (3F^{Cπ}) | Ac-DKWKAVYDKFAEAFKEFL-NH₂ | 112 |
| (3F^{Iπ}) | Ac-DKLKAFYDKVFEWAKEAF-NH₂ | 113 |

Other suitable class A peptides are characterized by having an improved hydrophobic face. Examples of such peptides are shown in Table 3.

TABLE 3
Illustrative peptides having an improved hydrophobic phase.

| | | SEQ |
|---|---|---|
| Name | Peptide | ID NO |
| V2W3A5F1017- | Ac-Asp-Val-Trp-Lys-Ala-Ala-Tyr-Asp-Lys-Phe- | 114 |
| D-4F | Ala-Glu-Lys-Phe-Lys-Glu-Phe-Phe-NH₂ | |
| V2W3F10-D-4F | Ac-Asp-Val-Trp-Lys-Ala-Phe-Tyr-Asp-Lys-Phe- | 115 |
| | Ala-Glu-Lys-Phe-Lys-Glu-Ala-Phe-NH₂ | |
| W3-D-4F | Ac-Asp-Phe-Trp-Lys-Ala-Phe-Tyr-Asp-Lys-Val- | 116 |
| | Ala-Glu-Lys-Phe-Lys-Glu-Ala-Phe-NH₂ | |
| | Ac-Phe-Phe-Glu-Lys-Phe-Lys-Glu-Ala-Phe-Lys- | 117 |
| | Asp-Tyr-Ala-Ala-Lys-Trp-Val-Asp-NH₂ | |
| | Ac-Phe-Als-Glu-Lys-Phe-Lys-Glu-Ala-Phe-Lys- | 118 |
| | Asp-Tyr-Phe-Ala-Lys-Trp-Val-Asp-NH₂ | |
| | Ac-Phe-Ala-Glu-Lys-Phe-Lys-Glu-Ala-Val-Lys- | 119 |
| | Asp-Tyr-Phe-Ala-Lys-Trp-Phe-Asp-NH₂ | |

The peptides described here (V2W3A5F10,17-D-4F; V2W3F10-D-4F; W3-D-4F) may be more potent than the original D-4F.

C) Smaller Peptides.

It was also a surprising discovery that certain small peptides consisting of a minimum of three amino acids preferentially (but not necessarily) with one or more of the amino acids being the D-stereoisomer of the amino acid, and possessing hydrophobic domains to permit lipid protein interactions, and hydrophilic domains to permit a degree of water solubility also possess significant anti-inflammatory properties and are useful in treating one ore more of the pathologies described herein. The “small peptides” typically range in length from 2 amino acids to about 15 amino acids, more preferably from about 3 amino acids to about 10 or 11 amino acids, and most preferably from about 4 to about 8 or 10 amino acids. In various embodiments the peptides are typically characterized by having hydrophobic terminal amino acids or terminal amino acids rendered hydrophobic by the attachment of one or more hydrophobic “protecting” groups. Various “small peptides” are described in copending applications U.S. Ser. No. 10/649,378, filed Aug. 26, 2003, and in U.S. Ser. No. 10/913,800, filed on Aug. 6, 2004, and in PCT Application PCT/US2004/026288.

In certain embodiments, the peptides can be characterized by Formula I, below:

| | | |
|---|---|---|
| X¹-X²-X³ₙ-X⁴ | I | (SEQ ID NO: 120) |

where, n is 0 or 1, X¹ is a hydrophobic amino acid and/or bears a hydrophobic protecting group, X⁴ is a hydrophobic amino acid and/or bears a hydrophobic protecting group; and when n is 0 X² is an acidic or a basic amino acid; when n is 1: X² and X³ are independently an acidic amino acid, a basic amino acid, an aliphatic amino acid, or an aromatic amino acid such that when X² is an acidic amino acid; X³ is a basic amino acid, an aliphatic amino acid, or an aromatic amino acid; when X² is a basic amino acid; X³ is an acidic amino acid, an aliphatic amino acid, or an aromatic amino acid; and when X² is an aliphatic or aromatic amino acid, X³ is an acidic amino acid, or a basic amino acid.

Longer peptides (e.g., up to 10, 11, or 15 amino acids) are also contemplated within the scope of this invention. Typically where the shorter peptides (e.g., peptides according to formula I) are characterized by an acidic, basic, aliphatic, or aromatic amino acid, the longer peptides are characterized by acidic, basic, aliphatic, or aromatic domains comprising two or more amino acids of that type.

1) Functional Properties of Active Small Peptides.

It was a surprising finding of this invention that a number of physical properties predict the ability of small peptides (e.g., less than 10 amino acids, preferably less than 8 amino acids, more preferably from about 3 to about 5 or 6 amino acids) of this invention to render HDL more anti-inflammatory and to mitigate atherosclerosis and/or other pathologies characterized by an inflammatory response in a mammal. The physical properties include high solubility in ethyl acetate (e.g., greater than about 4 mg/mL), and solubility in aqueous buffer at pH 7.0. Upon contacting phospholipids such as 1,2-Dimyristoyl-sn-glycero-3-phosphocholine (DMPC), in an aqueous environment, the particularly effective small peptides induce or participate in the formation of particles with a diameter of approximately 7.5 nm (±0.1 nm), and/or induce or participate in the formation of stacked bilayers with a bilayer dimension on the order of 3.4 to 4.1 nm with spacing between the bilayers in the stack of approximately 2 nm, and/or also induce or participate in the formation of vesicular structures of approximately 38 nm). In certain preferred embodiments, the small peptides have a molecular weight of less than about 900 Da.

Thus, in certain embodiments, this invention contemplates small peptides that ameliorate one or more symptoms of an indication/pathology described herein, e.g., an inflammatory condition, where the peptide(s): ranges in length from about 3 to about 8 amino acids, preferably from about 3 to about 6, or 7 amino acids, and more preferably from about 3 to about 5 amino acids; are soluble in ethyl acetate at a concentration greater than about 4 mg/mL; are soluble in aqueous buffer at pH 7.0; when contacted with a phospholipid in an aqueous environment, form particles with a diameter of approximately 7.5 nm and/or form stacked bilayers with a bilayer dimension on the order of 3.4 to 4.1 nm with spacing between the bilayers in the stack of approximately 2 nm; have a molecular weight less than about 900 daltons; convert pro-inflammatory HDL to anti-inflammatory HDL or make anti-inflammatory HDL more anti-inflammatory; and do not have the amino acid sequence Lys-Arg-Asp-Ser (SEQ ID NO:121), especially in which Lys-Arg-Asp and Ser are all L amino acids. In certain embodiments, these small peptides protect a phospholipid against oxidation by an oxidizing agent.

While these small peptides need not be so limited, in certain embodiments, these small peptides can include the small peptides described below.

2) Tripeptides.

It was discovered that certain tripeptides (3 amino acid peptides) can be synthesized that show desirable properties as described herein (e.g., the ability to convert pro-inflammatory HDL to anti-inflammatory HDL, the ability to decrease LDL-induced monocyte chemotactic activity generated by artery wall cells, the ability to increase pre-beta HDL, etc.). In certain embodiments, the peptides are characterized by formula I, wherein N is zero, shown below as Formula II:

X¹—X²—X⁴ II

where the end amino acids (X¹ and X⁴) are hydrophobic either because of a hydrophobic side chain or because the side chain or the C and/or N terminus is blocked with one or more hydrophobic protecting group(s) (e.g., the N-terminus is blocked with Boc-, Fmoc-, nicotinyl-, etc., and the C-terminus blocked with (tBu)-OtBu, etc.). In certain embodiments, the X² amino acid is either acidic (e.g., aspartic acid, glutamic acid, etc.) or basic (e.g., histidine, arginine, lysine, etc.). The peptide can be all L-amino acids or include one or more or all D-amino acids.

Certain preferred tripeptides of this invention include, but are not limited to the peptides shown in Table 4.

TABLE 4
Examples of certain preferred tripeptides bearing hydrophobic blocking
groups and acidic, basic, or histidine central amino acids.

| X¹ | X² X³ | X⁴ |
|---|---|---|
| Boc-Lys(εBoc) | Arg | Ser(tBu)-OtBu |
| Boc-Lys(εBoc) | Arg | Thr(tBu)-OtBu |
| Boc-Trp | Arg | Ile-OtBu |
| Boc-Trp | Arg | Leu-OtBu |
| Boc-Phe | Arg | Ile-OtBu |
| Boc-Phe | Arg | Leu-OtBu |
| Boc-Lys(εBoc) | Glu | Ser(tBu)-OtBu |
| Boc-Lys(εBoc) | Glu | Thr(tBu)-OtBu |
| Boc-Lys(εBoc) | Asp | Ser(tBu)-OtBu |
| Boc-Lys(εBoc) | Asp | Thr(tBu)-OtBu |
| Boc-Lys(εBoc) | Arg | Ser(tBu)-OtBu |
| Boc-Lys(εBoc) | Arg | Thr(tBu)-OtBu |
| Boc-Leu | Glu | Ser(tBu)-OtBu |
| Boc-Leu | Glu | Thr(tBu)-OtBu |
| Fmoc-Trp | Arg | Ser(tBu)-OtBu |
| Fmoc-Trp | Asp | Ser(tBu)-OtBu |
| Fmoc-Trp | Glu | Ser(tBu)-OtBu |
| Fmoc-Trp | Arg | Ser(tBu)-OtBu |
| Boc-Lys(εBoc) | Glu | Leu-OtBu |
| Fmoc-Leu | Arg | Ser(tBu)-OtBu |
| Fmoc-Leu | Asp | Ser(tBu)-OtBu |
| Fmoc-Leu | Glu | Ser(tBu)-OtBu |
| Fmoc-Leu | Arg | Ser(tBu)-OtBu |
| Fmoc-Leu | Arg | Thr(tBu)-OtBu |
| Boc-Glu | Asp | Tyr(tBu)-OtBu |
| Fmoc-Lys(εFmoc) | Arg | Ser(tBu)-OtBu |
| Fmoc-Trp | Arg | Ile-OtBu |
| Fmoc-Trp | Arg | Leu-OtBu |
| Fmoc-Phe | Arg | Ile-OtBu |
| Fmoc-Phe | Arg | Leu-OtBu |
| Boc-Trp | Arg | Phe-OtBu |
| Boc-Trp | Arg | Tyr-OtBu |
| Fmoc-Trp | Arg | Phe-OtBu |
| Fmoc-Trp | Arg | Tyr-OtBu |
| Boc-Orn(δBoc) | Arg | Ser(tBu)-OtBu |
| Nicotinyl Lys(εBoc) | Arg | Ser(tBu)-OtBu |
| Nicotinyl Lys(εBoc) | Arg | Thr(tBu)-OtBu |
| Fmoc-Leu | Asp | Thr(tBu)-OtBu |
| Fmoc-Leu | Glu | Thr(tBu)-OtBu |
| Fmoc-Leu | Arg | Thr(tBu)-OtBu |
| Fmoc-norLeu | Arg | Ser(tBu)-OtBu |
| Fmoc-norLeu | Asp | Ser(tBu)-OtBu |
| Fmoc-norLeu | Glu | Ser(tBu)-OtBu |
| Fmoc-Lys(εBoc) | Arg | Ser(tBu)-OtBu |
| Fmoc-Lys(εBoc) | Arg | Thr(tBu)-OtBu |
| Fmoc-Lys(εBoc) | Glu | Ser(tBu)-OtBu |
| Fmoc-Lys(εBoc) | Glu | Thr(tBu)-OtBu |
| Fmoc-Lys(εBoc) | Asp | Ser(tBu)-OtBu |
| Fmoc-Lys(εBoc) | Asp | Thr(tBu)-OtBu |
| Fmoc-Lys(εBoc) | Glu | Leu-OtBu |
| Fmoc-Lys(εBoc) | Arg | Leu-OtBu |
| Fmoc-Lys(εFmoc) | Arg | Thr(tBu)-OtBu |
| Fmoc- Lys(εFmoc) | Glu | Ser(tBu)-OtBu |
| Fmoc- Lys(εFmoc) | Glu | Thr(tBu)-OtBu |
| Fmoc- Lys(εFmoc) | Asp | Ser(tBu)-OtBu |
| Fmoc- Lys(εFmoc) | Asp | Thr(tBu)-OtBu |
| Fmoc- Lys(εFmoc) | Arg | Ser(tBu)-OtBu |
| Fmoc- Lys(εFmoc)) | Glu | Leu-OtBu |
| Boc-Lys(εFmoc) | Asp | Ser(tBu)-OtBu |
| Boc-Lys(εFmoc) | Asp | Thr(tBu)-OtBu |
| Boc-Lys(εFmoc) | Arg | Thr(tBu)-OtBu |
| Boc-Lys(εFmoc) | Glu | Leu-OtBu |
| Boc-Orn(δFmoc) | Glu | Ser(tBu)-OtBu |
| Boc-Orn(δFmoc) | Asp | Ser(tBu)-OtBu |
| Boc-Orn(δFmoc) | Asp | Thr(tBu)-OtBu |
| Boc-Orn(δFmoc) | Arg | Thr(tBu)-OtBu |
| Boc-Orn(δFmoc) | Glu | Thr(tBu)-OtBu |
| Fmoc-Trp | Asp | Ile-OtBu |
| Fmoc-Trp | Arg | Ile-OtBu |
| Fmoc-Trp | Glu | Ile-OtBu |
| Fmoc-Trp | Asp | Leu-OtBu |
| Fmoc-Trp | Glu | Leu-OtBu |
| Fmoc-Phe | Asp | Ile-OtBu |
| Fmoc-Phe | Asp | Leu-OtBu |
| Fmoc-Phe | Glu | Leu-OtBu |
| Fmoc-Trp | Arg | Phe-OtBu |
| Fmoc-Trp | Glu | Phe-OtBu |
| Fmoc-Trp | Asp | Phe-OtBu |
| Fmoc-Trp | Asp | Tyr-OtBu |
| Fmoc-Trp | Arg | Tyr-OtBu |
| Fmoc-Trp | Glu | Tyr-OtBu |
| Fmoc-Trp | Arg | Thr(tBu)-OtBu |
| Fmoc-Trp | Asp | Thr(tBu)-OtBu |
| Fmoc-Trp | Glu | Thr(tBu)-OtBu |
| Boc-Phe | Arg | norLeu-OtBu |
| Boc-Phe | Glu | norLeu-OtBu |
| Fmoc-Phe | Asp | norLeu-OtBu |
| Boc-Glu | His | Tyr(tBu)-OtBu |
| Boc-Leu | His | Ser(tBu)-OtBu |
| Boc-Leu | His | Thr(tBu)-OtBu |
| Boc-Lys(εBoc) | His | Ser(tBu)-OtBu |
| Boc-Lys(εBoc) | His | Thr(tBu)-OtBu |
| Boc-Lys(εBoc) | His | Leu-OtBu |
| Boc-Lys(εFmoc) | His | Ser(tBu)-OtBu |
| Boc-Lys(εFmoc) | His | Thr(tBu)-OtBu |
| Boc-Lys(εFmoc) | His | Leu-OtBu |
| Boc-Orn(δBoc) | His | Ser(tBu)-OtBu |
| Boc-Orn(δFmoc) | His | Thr(tBu)-OtBu |
| Boc-Phe | His | Ile-OtBu |
| Boc-Phe | His | Leu-OtBu |
| Boc-Phe | His | norLeu-OtBu |
| Boc-Phe | Lys | Leu-OtBu |
| Boc-Trp | His | Ile-OtBu |
| Boc-Trp | His | Leu-OtBu |
| Boc-Trp | His | Phe-OtBu |
| Boc-Trp | His | Tyr-OtBu |
| Boc-Phe | Lys | Leu-OtBu |
| Fmoc- Lys(εFmoc) | His | Ser(tBu)-OtBu |
| Fmoc- Lys(εFmoc) | His | Thr(tBu)-OtBu |
| Fmoc- Lys(εFmoc) | His | Leu-OtBu |
| Fmoc-Leu | His | Ser(tBu)-OtBu |
| Fmoc-Leu | His | Thr(tBu)-OtBu |
| Fmoc-Lys(εBoc) | His | Ser(tBu)-OtBu |
| Fmoc-Lys(εBoc) | His | Thr(tBu)-OtBu |
| Fmoc-Lys(εBoc) | His | Leu-OtBu |
| Fmoc-Lys(εFmoc) | His | Ser(tBu)-OtBu |
| Fmoc-Lys(εFmoc) | His | Thr(tBu)-OtBu |
| Fmoc-norLeu | His | Ser(tBu)-OtBu |
| Fmoc-Phe | His | Ile-OtBu |
| Fmoc-Phe | His | Leu-OtBu |
| Fmoc-Phe | His | norLeu-OtBu |
| Fmoc-Trp | His | Ser(tBu)-OtBu |
| Fmoc-Trp | His | Ile-OtBu |
| Fmoc-Trp | His | Leu-OtBu |
| Fmoc-Trp | His | Phe-OtBu |
| Fmoc-Trp | His | Tyr-OtBu |
| Fmoc-Trp | His | Thr(tBu)-OtBu |
| Nicotinyl Lys(εBoc) | His | Ser(tBu)-OtBu |
| Nicotinyl Lys(εBoc) | His | Thr(tBu)-OtBu |

While the peptides of Table 4 are illustrated with particular protecting groups, it is noted that these groups may be substituted with other protecting groups as described herein and/or one or more of the shown protecting group can be eliminated.

3) Small Peptides with Central Acidic and Basic Amino Acids.

In certain embodiments, the peptides of this invention range from four amino acids to about ten amino acids. The terminal amino acids are typically hydrophobic either because of a hydrophobic side chain or because the terminal amino acids bear one or more hydrophobic protecting groups end amino acids (X¹ and X⁴) are hydrophobic either because of a hydrophobic side chain or because the side chain or the C and/or N terminus is blocked with one or more hydrophobic protecting group(s) (e.g., the N-terminus is blocked with Boc-, Fmoc-, Nicotinyl-, etc., and the C-terminus blocked with (tBu)-OtBu, etc.). Typically, the central portion of the peptide comprises a basic amino acid and an acidic amino acid (e.g., in a 4 mer) or a basic domain and/or an acidic domain in a longer molecule.

These four-mers can be represented by Formula I in which X¹ and X⁴ are hydrophobic and/or bear hydrophobic protecting group(s) as described herein and X² is acidic while X³ is basic or X² is basic while X³ is acidic. The peptide can be all L-amino acids or include one or more or all D-amino acids.

Certain preferred of this invention include, but are not limited to the peptides shown in Table 5.

TABLE 5
Illustrative examples of small peptides with
central acidic and basic amino acids.

| | | | | SEQ ID |
|---|---|---|---|---|
| X¹ | X² | X³ | X⁴ | NO |
| Boc-Lys(εBoc) | Arg | Asp | Ser(tBu)-OtBu | 121 |
| Boc-Lys(εBoc) | Arg | Asp | Thr(tBu)-OtBu | 122 |
| Boc-Trp | Arg | Asp | Ile-OtBu | 123 |
| Boc-Trp | Arg | Asp | Leu-OtBu | 124 |
| Boc-Phe | Arg | Asp | Leu-OtBu | 125 |
| Boc-Phe | Arg | Asp | Ile-OtBu | 126 |
| Boc-Phe | Arg | Asp | norLeu-OtBu | 127 |
| Boc-Phe | Arg | Glu | norLeu-OtBu | 128 |
| Boc-Phe | Arg | Glu | Ile-OtBu | 129 |
| Boc-Phe | Asp | Arg | Ile-OtBu | 130 |
| Boc-Phe | Glu | Arg | Ile-OtBu | 131 |
| Boc-Phe | Asp | Arg | Leu-OtBu | 132 |
| Boc-Phe | Arg | Glu | Leu-OtBu | 133 |
| Boc-Phe | Glu | Arg | Leu-OtBu | 134 |
| Boc-Phe | Asp | Arg | norLeu-OtBu | 135 |
| Boc-Phe | Glu | Arg | norLeu-OtBu | 136 |
| Boc-Lys(εBoc) | Glu | Arg | Ser(tBu)-OtBu | 137 |
| Boc-Lys(εBoc) | Glu | Arg | Thr(tBu)-OtBu | 138 |
| Boc-Lys(εBoc) | Asp | Arg | Ser(tBu)-OtBu | 139 |
| Boc-Lys(εBoc) | Asp | Arg | Thr(tBu)-OtBu | 140 |
| Boc-Lys(εBoc) | Arg | Glu | Ser(tBu)-OtBu | 141 |
| Boc-Lys(εBoc) | Arg | Glu | Thr(tBu)-OtBu | 142 |
| Boc-Leu | Glu | Arg | Ser(tBu)-OtBu | 143 |
| Boc-Leu | Glu | Arg | Thr(tBu)-OtBu | 144 |
| Fmoc-Trp | Arg | Asp | Ser(tBu)-OtBu | 145 |
| Fmoc-Trp | Asp | Arg | Ser(tBu)-OtBu | 146 |
| Fmoc-Trp | Glu | Arg | Ser(tBu)-OtBu | 147 |
| Fmoc-Trp | Arg | Glu | Ser(tBu)-OtBu | 148 |
| Boc-Lys(εBoc) | Glu | Arg | Leu-OtBu | 149 |
| Fmoc-Leu | Arg | Asp | Ser(tBu)-OtBu | 150 |
| Fmoc-Leu | Asp | Arg | Ser(tBu)-OtBu | 151 |
| Fmoc-Leu | Glu | Arg | Ser(tBu)-OtBu | 152 |
| Fmoc-Leu | Arg | Glu | Ser(tBu)-OtBu | 153 |
| Fmoc-Leu | Arg | Asp | Thr(tBu)-OtBu | 154 |
| Boc-Glu | Asp | Arg | Tyr(tBu)-OtBu | 155 |
| Fmoc-Lys(εFmoc) | Arg | Asp | Ser(tBu)-OtBu | 156 |
| Fmoc-Trp | Arg | Asp | Ile-OtBu | 157 |
| Fmoc-Trp | Arg | Asp | Leu-OtBu | 158 |
| Fmoc-Phe | Arg | Asp | Ile-OtBu | 159 |
| Fmoc-Phe | Arg | Asp | Leu-OtBu | 160 |
| Boc-Trp | Arg | Asp | Phe-OtBu | 161 |
| Boc-Trp | Arg | Asp | Tyr-OtBu | 162 |
| Fmoc-Trp | Arg | Asp | Phe-OtBu | 163 |
| Fmoc-Trp | Arg | Asp | Tyr-OtBu | 164 |
| Boc-Orn(δBoc) | Arg | Glu | Ser(tBu)-OtBu | 165 |
| Nicotinyl Lys(εBoc) | Arg | Asp | Ser(tBu)-OtBu | 166 |
| Nicotinyl Lys(εBoc) | Arg | Asp | Thr(tBu)-OtBu | 167 |
| Fmoc-Leu | Asp | Arg | Thr(tBu)-OtBu | 168 |
| Fmoc-Leu | Glu | Arg | Thr(tBu)-OtBu | 169 |
| Fmoc-Leu | Arg | Glu | Thr(tBu)-OtBu | 170 |
| Fmoc-norLeu | Arg | Asp | Ser(tBu)-OtBu | 171 |
| Fmoc-norLeu | Asp | Arg | Ser(tBu)-OtBu | 172 |
| Fmoc-norLeu | Glu | Arg | Ser(tBu)-OtBu | 173 |
| Fmoc-norLeu | Arg | Glu | Ser(tBu)-OtBu | 174 |
| Fmoc-Lys(εBoc) | Arg | Asp | Ser(tBu)-OtBu | 175 |
| Fmoc-Lys(εBoc) | Arg | Asp | Thr(tBu)-OtBu | 176 |
| Fmoc-Lys(εBoc) | Glu | Arg | Ser(tBu)-OtBu | 177 |
| Fmoc-Lys(εBoc) | Glu | Arg | Thr(tBu)-OtBu | 178 |
| Fmoc-Lys(εBoc) | Asp | Arg | Ser(tBu)-OtBu | 179 |
| Fmoc-Lys(εBoc) | Asp | Arg | Thr(tBu)-OtBu | 180 |
| Fmoc-Lys(εBoc) | Arg | Glu | Ser(tBu)-OtBu | 181 |
| Fmoc-Lys(εBoc) | Arg | Glu | Thr(tBu)-OtBu | 182 |
| Fmoc-Lys(εBoc) | Glu | Arg | Leu-OtBu | 183 |
| Fmoc-Lys(εBoc) | Arg | Glu | Leu-OtBu | 184 |
| Fmoc-Lys(εFmoc) | Arg | Asp | Thr(tBu)-OtBu | 185 |
| Fmoc- Lys(εFmoc) | Glu | Arg | Ser(tBu)-OtBu | 186 |
| Fmoc- Lys(εFmoc) | Glu | Arg | Thr(tBu)-OtBu | 187 |
| Fmoc- Lys(εFmoc) | Asp | Arg | Ser(tBu)-OtBu | 188 |
| Fmoc- Lys(εFmoc) | Asp | Arg | Thr(tBu)-OtBu | 189 |
| Fmoc- Lys(εFmoc) | Arg | Glu | Ser(tBu)-OtBu | 190 |
| Fmoc- Lys(εFmoc) | Arg | Glu | Thr(tBu)-OtBu | 191 |
| Fmoc- Lys(εFmoc)) | Glu | Arg | Leu-OtBu | 192 |
| Boc-Lys(εFmoc) | Arg | Asp | Ser(tBu)-OtBu | 193 |
| Boc-Lys(εFmoc) | Arg | Asp | Thr(tBu)-OtBu | 194 |
| Boc-Lys(εFmoc) | Glu | Arg | Ser(tBu)-OtBu | 195 |
| Boc-Lys(εFmoc) | Glu | Arg | Thr(tBu)-OtBu | 196 |
| Boc-Lys(εFmoc) | Asp | Arg | Ser(tBu)-OtBu | 197 |
| Boc-Lys(εFmoc) | Asp | Arg | Thr(tBu)-OtBu | 198 |
| Boc-Lys(εFmoc) | Arg | Glu | Ser(tBu)-OtBu | 199 |
| Boc-Lys(εFmoc) | Arg | Glu | Thr(tBu)-OtBu | 200 |
| Boc-Lys(εFmoc) | Glu | Arg | Leu-OtBu | 201 |
| Boc-Orn(δFmoc) | Arg | Glu | Ser(tBu)-OtBu | 202 |
| Boc-Orn(δFmoc) | Glu | Arg | Ser(tBu)-OtBu | 203 |
| Boc-Orn(δFmoc) | Arg | Asp | Ser(tBu)-OtBu | 204 |
| Boc-Orn(δFmoc) | Asp | Arg | Ser(tBu)-OtBu | 205 |
| Boc-Orn(δFmoc) | Asp | Arg | Thr(tBu)-OtBu | 206 |
| Boc-Orn(δFmoc) | Arg | Asp | Thr(tBu)-OtBu | 207 |
| Boc-Orn(δFmoc) | Glu | Arg | Thr(tBu)-OtBu | 208 |
| Boc-Orn(δFmoc) | Arg | Glu | Thr(tBu)-OtBu | 209 |
| Fmoc-Trp | Asp | Arg | Ile-OtBu | 210 |
| Fmoc-Trp | Arg | Glu | Ile-OtBu | 211 |
| Fmoc-Trp | Glu | Arg | Ile-OtBu | 212 |
| Fmoc-Trp | Asp | Arg | Leu-OtBu | 213 |
| Fmoc-Trp | Arg | Glu | Leu-OtBu | 214 |
| Fmoc-Trp | Glu | Arg | Leu-OtBu | 215 |
| Fmoc-Phe | Asp | Arg | Ile-OtBu | 216 |
| Fmoc-Phe | Arg | Glu | Ile-OtBu | 217 |
| Fmoc-Phe | Glu | Arg | Ile-OtBu | 218 |
| Fmoc-Phe | Asp | Arg | Leu-OtBu | 219 |
| Fmoc-Phe | Arg | Glu | Leu-OtBu | 220 |
| Fmoc-Phe | Glu | Arg | Leu-OtBu | 221 |
| Fmoc-Trp | Arg | Asp | Phe-OtBu | 222 |
| Fmoc-Trp | Arg | Glu | Phe-OtBu | 223 |
| Fmoc-Trp | Glu | Arg | Phe-OtBu | 224 |
| Fmoc-Trp | Asp | Arg | Tyr-OtBu | 225 |
| Fmoc-Trp | Arg | Glu | Tyr-OtBu | 226 |
| Fmoc-Trp | Glu | Arg | Tyr-OtBu | 227 |
| Fmoc-Trp | Arg | Asp | Thr(tBu)-OtBu | 228 |
| Fmoc-Trp | Asp | Arg | Thr(tBu)-OtBu | 229 |
| Fmoc-Trp | Arg | Glu | Thr(tBu)-OtBu | 230 |
| Fmoc-Trp | Glu | Arg | Thr(tBu)-OtBu | 231 |
| Fmoc-Phe | Arg | Asp | norLeu-OtBu | 232 |
| Fmoc-Phe | Arg | Glu | norLeu-OtBu | 233 |
| Boc-Phe | Lys | Asp | Leu-OtBu | 234 |
| Boc-Phe | Asp | Lys | Leu-OtBu | 235 |
| Boc-Phe | Lys | Glu | Leu-OtBu | 236 |
| Boc-Phe | Glu | Lys | Leu-OtBu | 237 |
| Boc-Phe | Lys | Asp | Ile-OtBu | 238 |
| Boc-Phe | Asp | Lys | Ile-OtBu | 239 |
| Boc-Phe | Lys | Glu | Ile-OtBu | 240 |
| Boc-Phe | Glu | Lys | Ile-OtBu | 241 |
| Boc-Phe | Lys | Asp | norLeu-OtBu | 242 |
| Boc-Phe | Asp | Lys | norLeu-OtBu | 243 |
| Boc-Phe | Lys | Glu | norLeu-OtBu | 244 |
| Boc-Phe | Glu | Lys | norLeu-OtBu | 245 |
| Boc-Phe | His | Asp | Leu-OtBu | 246 |
| Boc-Phe | Asp | His | Leu-OtBu | 247 |
| Boc-Phe | His | Glu | Leu-OtBu | 248 |
| Boc-Phe | Glu | His | Leu-OtBu | 249 |
| Boc-Phe | His | Asp | Ile-OtBu | 250 |
| Boc-Phe | Asp | His | Ile-OtBu | 251 |
| Boc-Phe | His | Glu | Ile-OtBu | 252 |
| Boc-Phe | Glu | His | Ile-OtBu | 253 |
| Boc-Phe | His | Asp | norLeu-OtBu | 254 |
| Boc-Phe | Asp | His | norLeu-OtBu | 255 |
| Boc-Phe | His | Glu | norLeu-OtBu | 256 |
| Boc-Phe | Glu | His | norLeu-OtBu | 257 |
| Boc-Lys(εBoc) | Lys | Asp | Ser(tBu)-OtBu | 258 |
| Boc-Lys(εBoc) | Asp | Lys | Ser(tBu)-OtBu | 259 |
| Boc-Lys(εBoc) | Lys | Glu | Ser(tBu)-OtBu | 260 |
| Boc-Lys(εBoc) | Glu | Lys | Ser(tBu)-OtBu | 261 |
| Boc-Lys(εBoc) | His | Asp | Ser(tBu)-OtBu | 262 |
| Boc-Lys(εBoc) | Asp | His | Ser(tBu)-OtBu | 263 |
| Boc-Lys(εBoc) | His | Glu | Ser(tBu)-OtBu | 264 |
| Boc-Lys(εBoc) | Glu | His | Ser(tBu)-OtBu | 265 |

While the pepides of Table 5 are illustrated with particular protecting groups, it is noted that these groups may be substituted with other protecting groups as described herein and/or one or more of the shown protecting group can be eliminated.

4) Small Peptides Having Either an Acidic or Basic Amino Acid in the Center Together with a Central Aliphatic Amino Acid.

In certain embodiments, the peptides of this invention range from four amino acids to about ten amino acids. The terminal amino acids are typically hydrophobic either because of a hydrophobic side chain or because the terminal amino acids bear one or more hydrophobic protecting groups. End amino acids (X¹ and X⁴) are hydrophobic either because of a hydrophobic side chain or because the side chain or the C and/or N terminus is blocked with one or more hydrophobic protecting group(s) (e.g., the N-terminus is blocked with Boc-, Fmoc-, Nicotinyl-, etc., and the C-terminus blocked with (tBu)-OtBu, etc.). Typically, the central portion of the peptide comprises a basic or acidic amino acid and an aliphatic amino acid (e.g., in a 4 mer) or a basic domain or an acidic domain and an aliphatic domain in a longer molecule.

These four-mers can be represented by Formula I in which X¹ and X⁴ are hydrophobic and/or bear hydrophobic protecting group(s) as described herein and X² is acidic or basic while X³ is aliphatic or X² is aliphatic while X³ is acidic or basic. The peptide can be all L-amino acids or include one, or more, or all D-amino acids.

Certain preferred peptides of this invention include, but are not limited to the peptides shown in Table 6.

TABLE 6
Examples of certain preferred peptides having either
an acidic or basic amino acid in the center together
with a central aliphatic amino acid.

| | | | | SEQ ID |
|---|---|---|---|---|
| X¹ | X² | X³ | X⁴ | NO |
| Fmoc-Lys(εBoc) | Leu | Arg | Ser(tBu)-OtBu | 266 |
| Fmoc-Lys(εBoc) | Arg | Leu | Ser(tBu)-OtBu | 267 |
| Fmoc-Lys(εBoc) | Leu | Arg | Thr(tBu)-OtBu | 268 |
| Fmoc-Lys(εBoc) | Arg | Leu | Thr(tBu)-OtBu | 269 |
| Fmoc-Lys(εBoc) | Glu | Leu | Ser(tBu)-OtBu | 270 |
| Fmoc-Lys(εBoc) | Leu | Glu | Ser(tBu)-OtBu | 271 |
| Fmoc-Lys(εBoc) | Glu | Leu | Thr(tBu)-OtBu | 272 |
| Fmoc-Lys(εBoc) | Leu | Glu | Thr(tBu)-OtBu | 273 |
| Fmoc- Lys(εFmoc) | Leu | Arg | Ser(tBu)-OtBu | 274 |
| Fmoc- Lys(εFmoc) | Leu | Arg | Thr(tBu)-OtBu | 275 |
| Fmoc- Lys(εFmoc) | Glu | Leu | Ser(tBu)-OtBu | 276 |
| Fmoc- Lys(εFmoc) | Glu | Leu | Thr(tBu)-OtBu | 277 |
| Boc-Lys(Fmoc) | Glu | Ile | Thr(tBu)-OtBu | 278 |
| Boc-Lys(εFmoc) | Leu | Arg | Ser(tBu)-OtBu | 279 |
| Boc-Lys(εFmoc) | Leu | Arg | Thr(tBu)-OtBu | 280 |
| Boc-Lys(εFmoc) | Glu | Leu | Ser(tBu)-OtBu | 281 |
| Boc-Lys(εFmoc) | Glu | Leu | Thr(tBu)-OtBu | 282 |
| Boc-Lys(εBoc) | Leu | Arg | Ser(tBu)-OtBu | 283 |
| Boc-Lys(εBoc) | Arg | Phe | Thr(tBu)-OtBu | 284 |
| Boc-Lys(εBoc) | Leu | Arg | Thr(tBu)-OtBu | 285 |
| Boc-Lys(εBoc) | Glu | Ile | Thr(tBu) | 286 |
| Boc-Lys(εBoc) | Glu | Val | Thr(tBu) | 287 |
| Boc-Lys(εBoc) | Glu | Ala | Thr(tBu) | 288 |
| Boc-Lys(εBoc) | Glu | Gly | Thr(tBu) | 289 |
| Boc--Lys(εBoc) | Glu | Leu | Ser(tBu)-OtBu | 290 |
| Boc-Lys(εBoc) | Glu | Leu | Thr(tBu)-OtBu | 291 |

While the pepides of Table 6 are illustrated with particular protecting groups, it is noted that these groups may be substituted with other protecting groups as described herein and/or one or more of the shown protecting group can be eliminated.

5) Small Peptides Having Either an Acidic or Basic Amino Acid in the Center Together with a Central Aromatic Amino Acid.

In certain embodiments, the “small” peptides of this invention range from four amino acids to about ten amino acids. The terminal amino acids are typically hydrophobic either because of a hydrophobic side chain or because the terminal amino acids bear one or more hydrophobic protecting groups end amino acids (X¹ and X⁴) are hydrophobic either because of a hydrophobic side chain or because the side chain or the C and/or N terminus is blocked with one or more hydrophobic protecting group(s) (e.g., the N-terminus is blocked with Boc-, Fmoc-, Nicotinyl-, etc., and the C-terminus blocked with (tBu)-OtBu, etc.). Typically, the central portion of the peptide comprises a basic or acidic amino acid and an aromatic amino acid (e.g., in a 4 mer) or a basic domain or an acidic domain and an aromatic domain in a longer molecule.

These four-mers can be represented by Formula I in which X¹ and X⁴ are hydrophobic and/or bear hydrophobic protecting group(s) as described herein and X² is acidic or basic while X³ is aromatic or X² is aromatic while X³ is acidic or basic. The peptide can be all L-amino acids or include one, or more, or all D-amino acids. Five-mers can be represented by a minor modification of Formula I in which X⁵ is inserted as shown in Table 7 and in which X⁵ is typically an aromatic amino acid.

Certain preferred peptides of this invention include, but are not limited to the peptides shown in Table 7.

TABLE 7
Examples of certain preferred peptides having either
an acidic or basic amino acid in the center together
with a central aromatic amino acid.

| | | | | | SEQ |
|---|---|---|---|---|---|
| | | | | | ID |
| X¹ | X² | X³ | X⁵ | X⁴ | NO |
| Fmoc-Lys(εBoc) | Arg | Trp | | Tyr(tBu)-OtBu | 292 |
| Fmoc-Lys(εBoc) | Trp | Arg | | Tyr(tBu)-OtBu | 293 |
| Fmoc-Lys(εBoc) | Arg | Tyr | | Trp-OtBu | 294 |
| Fmoc-Lys(εBoc) | Tyr | Arg | | Trp-OtBu | 295 |
| Fmoc-Lys(εBoc) | Arg | Tyr | Trp | Thr(tBu)-OtBu | 296 |
| Fmoc-Lys(εBoc) | Arg | Tyr | | Thr(tBu)-OtBu | 297 |
| Fmoc-Lys(εBoc) | Arg | Trp | | Thr(tBu)-OtBu | 298 |
| Fmoc- Lys(εFmoc) | Arg | Trp | | Tyr(tBu)-OtBu | 299 |
| Fmoc- Lys(εFmoc) | Arg | Tyr | | Trp-OtBu | 300 |
| Fmoc- Lys(εFmoc) | Arg | Tyr | Trp | Thr(tBu)-OtBu | 301 |
| Fmoc- Lys(εFmoc) | Arg | Tyr | | Thr(tBu)-OtBu | 302 |
| Fmoc- Lys(εFmoc) | Arg | Trp | | Thr(tBu)-OtBu | 303 |
| Boc-Lys(εFmoc) | Arg | Trp | | Tyr(tBu)-OtBu | 304 |
| Boc-Lys(εFmoc) | Arg | Tyr | | Trp-OtBu | 305 |
| Boc-Lys(εFmoc) | Arg | Tyr | Trp | Thr(tBu)-OtBu | 306 |
| Boc-Lys(εFmoc) | Arg | Tyr | | Thr(tBu)-OtBu | 307 |
| Boc-Lys(εFmoc) | Arg | Trp | | Thr(tBu)-OtBu | 308 |
| Boc-Glu | Lys(εFmoc) | Arg | | Tyr(tBu)-OtBu | 309 |
| Boc-Lys(εBoc) | Arg | Trp | | Tyr(tBu)-OtBu | 310 |
| Boc-Lys(εBoc) | Arg | Tyr | | Trp-OtBu | 311 |
| Boc-Lys(εBoc) | Arg | Tyr | Trp | Thr(tBu)-OtBu | 312 |
| Boc-Lys(εBoc) | Arg | Tyr | | Thr(tBu)-OtBu | 313 |
| Boc-Lys(εBoc) | Arg | Phe | | Thr(tBu)-OtBu | 314 |
| Boc-Lys(εBoc) | Arg | Trp | | Thr(tBu)-OtBu | 315 |

While the peptides of Table 7 are illustrated with particular protecting groups, it is noted that these groups may be substituted with other protecting groups as described herein and/or one or more of the shown protecting group can be eliminated.

6) Small Peptides Having Aromatic Amino Acids or Aromatic Amino Acids Separated by Histidine(s) at the Center.

In certain embodiments, the peptides of this invention are characterized by π electrons that are exposed in the center of the molecule which allow hydration of the particle and that allow the peptide particles to trap pro-inflammatory oxidized lipids such as fatty acid hydroperoxides and phospholipids that contain an oxidation product of arachidonic acid at the sn-2 position.

In certain embodiments, these peptides consist of a minimum of 4 amino acids and a maximum of about 10 amino acids, preferentially (but not necessarily) with one or more of the amino acids being the D-sterioisomer of the amino acid, with the end amino acids being hydrophobic either because of a hydrophobic side chain or because the terminal amino acid(s) bear one or more hydrophobic blocking group(s), (e.g., an N-terminus blocked with Boc-, Fmoc-, Nicotinyl-, and the like, and a C-terminus blocked with (tBu)-OtBu groups and the like). Instead of having an acidic or basic amino acid in the center, these peptides generally have an aromatic amino acid at the center or have aromatic amino acids separated by histidine in the center of the peptide.

Certain preferred peptides of this invention include, but are not limited to the peptides shown in Table 8.

TABLE 8
Examples of peptides having aromatic amino acids
in the center or aromatic amino acids or aromatic
domains separated by one or more histidines.

| | | | | | SEQ ID |
|---|---|---|---|---|---|
| X¹ | X² | X³ | X⁴ | X⁵ | NO |
| Boc-Lys(εBoc) | Phe | Trp | Phe | Ser(tBu)-OtBu | 316 |
| Boc-Lys(εBoc) | Phe | Trp | Phe | Thr(tBu)-OtBu | 317 |
| Boc-Lys(εBoc) | Phe | Tyr | Phe | Ser(tBu)-OtBu | 318 |
| Boc-Lys(εBoc) | Phe | Tyr | Phe | Thr(tBu)-OtBu | 319 |
| Boc-Lys(εBoc) | Phe | His | Phe | Ser(tBu)-OtBu | 320 |
| Boc-Lys(εBoc) | Phe | His | Phe | Thr(tBu)-OtBu | 321 |
| Boc-Lys(εBoc) | Val | Phe | Phe-Tyr | Ser(tBu)-OtBu | 322 |
| Nicotinyl-Lys(εBoc) | Phe | Trp | Phe | Ser(tBu)-OtBu | 323 |
| Nicotinyl-Lys(εBoc) | Phe | Trp | Phe | Thr(tBu)-OtBu | 324 |
| Nicotinyl-Lys(εBoc) | Phe | Tyr | Phe | Ser(tBu)-OtBu | 325 |
| Nicotinyl-Lys(εBoc) | Phe | Tyr | Phe | Thr(tBu)-OtBu | 326 |
| Nicotinyl-Lys(εBoc) | Phe | His | Phe | Ser(tBu)-OtBu | 327 |
| Nicotinyl-Lys(εBoc) | Phe | His | Phe | Thr(tBu)-OtBu | 328 |
| Boc-Leu | Phe | Trp | Phe | Thr(tBu)-OtBu | 329 |
| Boc-Leu | Phe | Trp | Phe | Ser(tBu)-OtBu | 330 |

While the peptides of Table 8 are illustrated with particular protecting groups, it is noted that these groups may be substituted with other protecting groups as described herein and/or one or more of the shown protecting group can be eliminated.

7) Summary of Tripeptides and Tetrapeptides.

For the sake of clarity, a number of tripeptides and tetrapeptides of this invention are generally summarized below in Table 9.

TABLE 9
General structure of certain peptides of this invention.

| X¹ | X² | X³ | X⁴ |
|---|---|---|---|
| hydrophobic side chain | Acidic | — | hydrophobic side |
| or hydrophobic | or Basic | | chain or |
| protecting group(s) | | | hydrophobic |
| | | | protecting group(s) |
| hydrophobic side chain | Basic | Acidic | hydrophobic side |
| or hydrophobic | | | chain or |
| protecting group(s) | | | hydrophobic |
| | | | protecting group(s) |
| hydrophobic side chain | Acidic | Basic | hydrophobic side |
| or hydrophobic | | | chain or |
| protecting group(s) | | | hydrophobic |
| | | | protecting group(s) |
| hydrophobic side chain | Acidic | Aliphatic | hydrophobic side |
| or hydrophobic | or Basic | | chain or |
| protecting group(s) | | | hydrophobic |
| | | | protecting group(s) |
| hydrophobic side chain | Aliphatic | Acidic | hydrophobic side |
| or hydrophobic | | or Basic | chain or |
| protecting group(s) | | | hydrophobic |
| | | | protecting group(s) |
| hydrophobic side chain | Acidic | Aromatic | hydrophobic side |
| or hydrophobic | or Basic | | chain or |
| protecting group(s) | | | hydrophobic |
| | | | protecting group(s) |
| hydrophobic side chain | Aromatic | Acidic | hydrophobic side |
| or hydrophobic | | or Basic | chain or |
| protecting group(s) | | | hydrophobic |
| | | | protecting group(s) |
| hydrophobic side chain | Aromatic | His | hydrophobic side |
| or hydrophobic | | Aromatic | chain or |
| protecting group(s) | | | hydrophobic |
| | | | protecting group(s) |

Where longer peptides are desired, X² and X³ can represent domains (e.g., regions of two or more amino acids of the specified type) rather than individual amino acids. Table 9 is intended to be illustrative and not limiting. Using the teaching provided herein, other suitable peptides can readily be identified.

8) Paired Amino Acids and Dipeptides.

In certain embodiments, this invention pertains to the discovery that certain pairs of amino acids, administered in conjunction with each other or linked to form a dipeptide have one or more of the properties described herein. Thus, without being bound to a particular theory, it is believed that when the pairs of amino acids are administered in conjunction with each other, as described herein, they are capable participating in or inducing the formation of micelles in vivo.

Similar to the other small peptides described herein, it is believed that the pairs of peptides will associate in vivo, and demonstrate physical properties including high solubility in ethyl acetate (e.g., greater than about 4 mg/mL), solubility in aqueous buffer at pH 7.0. Upon contacting phospholipids such as 1,2-Dimyristoyl-sn-glycero-3-phosphocholine (DMPC), in an aqueous environment, it is believed the pairs of amino acids induce or participate in the formation of particles with a diameter of approximately 7.5 nm (±0.1 nm), and/or induce or participate in the formation of stacked bilayers with a bilayer dimension on the order of 3.4 to 4.1 nm with spacing between the bilayers in the stack of approximately 2 nm, and/or also induce or participate in the formation of vesicular structures of approximately 38 nm).

Moreover, it is further believed that the pairs of amino acids can display one or more of the following physiologically relevant properties:
1. They convert pro-inflammatory HDL to anti-inflammatory HDL or make anti-inflammatory HDL more anti-inflammatory;
2. They decrease LDL-induced monocyte chemotactic activity generated by artery wall cells;
3. They stimulate the formation and cycling of pre-β HDL;
4. They raise HDL cholesterol; and/or
5. They increase HDL paraoxonase activity.

The pairs of amino acids can be administered as separate amino acids (administered sequentially or simultaneously, e.g. in a combined formulation) or they can be covalently coupled directly or through a linker (e.g. a PEG linker, a carbon linker, a branched linker, a straight chain linker, a heterocyclic linker, a linker formed of derivatized lipid, etc.). In certain embodiments, the pairs of amino acids are covalently linked through a peptide bond to form a dipeptide. In various embodiments while the dipeptides will typically comprise two amino acids each bearing an attached protecting group, this invention also contemplates dipeptides wherein only one of the amino acids bears one or more protecting groups.

The pairs of amino acids typically comprise amino acids where each amino acid is attached to at least one protecting group (e.g., a hydrophobic protecting group as described herein). The amino acids can be in the D or the L form. In certain embodiments, where the amino acids comprising the pairs are not attached to each other, each amino acid bears two protecting groups (e.g., such as molecules 1 and 2 in Table 10).

TABLE 10
Illustrative amino acid pairs of this invention.

| | Amino Acid Pair/Dipeptide |
|---|---|
| 1. | Boc-Arg-OtBu* |
| 2. | Boc-Glu-OtBu* |
| 3. | Boc-Phe-Arg-OtBu** |
| 4. | Boc-Glu-Leu-OtBu** |
| 5. | Boc-Arg-Glu-OtBu*** |

*This would typically be administered in
conjunciton with a second amino acid.
**In certain embodiments, these dipeptides would
be administered in conjunction with each other.
***In certain embodiments, this peptide would be
administered either alone or in combination with
one of the other peptides described herein..

Suitable pairs of amino acids can readily be identified by providing the pair of protected amino acids and/or a dipeptide and then screening the pair of amino acids/dipeptide for one or more of the physical and/or physiological properties described above. In certain embodiments, this invention excludes pairs of amino acids and/or dipeptides comprising aspartic acid and phenylalanine In certain embodiments, this invention excludes pairs of amino acids and/or dipeptides in which one amino acid is (−)-N-[trans-4-isopropylcyclohexane)carbonyl]-D-phenylalanine (nateglinide).

In certain embodiments, the amino acids comprising the pair are independently selected from the group consisting of an acidic amino acid (e.g., aspartic acid, glutamic acid, etc.), a basic amino acid (e.g., lysine, arginine, histidine, etc.), and a non-polar amino acid (e.g., alanine, valine, leucine, isoleucine, proline, phenylalanine, tryptophan, methionine, etc.). In certain embodiments, where the first amino acid is acidic or basic, the second amino acid is non-polar and where the second amino acid is acidic or basic, the first amino acid is non-polar. In certain embodiments, where the first amino acid is acidic, the second amino acid is basic, and vice versa. (see, e.g., Table 11).

Similar combinations can be obtained by administering pairs of dipeptides. Thus, for example in certain embodiments, molecules 3 and 4 in Table 10 would be administered in conjunction with each other.

TABLE 11
Certain generalized amino acid pairs/dipeptides.

| | First Amino acid | Second Amino acid |
|---|---|---|
| 1. | Acidic | Basic |
| 2. | Basic | Acidic |
| 3. | Acidic | Non-polar |
| 4. | Non-polar | Acidic |
| 5. | Basic | Non-polar |
| 6. | Non-polar | Basic |

It is noted that these amino acid pairs/dipeptides are intended to be illustrative and not limiting. Using the teaching provided herein other suitable amino acid pairs/dipeptides can readily be determined.

D) Apo-J (G* Peptides).

In certain It was a discovery of this invention that peptides that mimicking the amphipathic helical domains of apo J (e.g., various apo-M derivatives) are particularly effective in protecting LDL against oxidation by arterial wall cells and in reducing LDL-induced monocyte chemotactic activity that results from the oxidation of LDL by human artery wall cells, and are capable of mitigating one or more symptoms of atherosclerosis and/or other pathologies described herein.

Apolipoprotein J possesses a wide nonpolar face termed globular protein-like, or G* amphipathic helical domains. The class G amphipathic helix is found in globular proteins, and thus, the name class G. This class of amphipathic helix is characterized by a random distribution of positively charged and negatively charged residues on the polar face with a narrow nonpolar face. Because of the narrow nonpolar face this class does not readily associate with phospholipid (see Segrest et al. (1990) Proteins: Structure, Function, and Genetics. 8: 103-117; also see Erratum (1991) Proteins: Structure, Function and Genetics, 9: 79). Several exchangeable apolipoproteins possess similar but not identical characteristics to the G amphipathic helix. Similar to the class G amphipathic helix, this other class possesses a random distribution of positively and negatively charged residues on the polar face. However, in contrast to the class G amphipathic helix which has a narrow nonpolar face, this class has a wide nonpolar face that allows this class to readily bind phospholipid and the class is termed G* to differentiate it from the G class of amphipathic helix (see Segrest et al. (1992) J. Lipid Res., 33: 141-166; also see Anantharamaiah et al. (1993) Pp. 109-142 In The Amphipathic Helix, Epand, R. M. Ed., CRC Press, Boca Raton, Fla.).

A number of suitable G* amphipathic peptides are described in copending applications U.S. Ser. No. 10/120,508, filed Apr. 5, 2002, U.S. Ser. No. 10/520,207, filed Apr. 1, 2003, and PCT Application PCT/US03/09988, filed Apr. 1, 2003. In addition, a variety of suitable peptides of this invention that are related to G* amphipathic helical domains of apo J are illustrated in Table 12.

TABLE 12
Preferred peptides for use in this
invention related to G* amphipathic
helical domains of apo J.

| | SEQ |
|---|---|
| | ID |
| Amino Acid Sequence | NO |
| LLEQLNEQFNWVSRLANLTQGE | 331 |
| LLEQLNEQFNWVSRLANL | 332 |
| NELQEMSNQGSKYVNKEIQNAVNGV | 333 |
| IQNAVNGVKQIKTLIEKTNEE | 334 |
| RKTLLSNLEEAKKKKEDALNETRESETKLKEL | 335 |
| PGVCNETMMALWEECK | 336 |
| PCLKQTCMKFYARVCR | 337 |
| ECKPCLKQTCMKFYARVCR | 338 |
| LVGRQLEEFL | 339 |
| MNGDRIDSLLEN | 340 |
| QQTHMLDVMQD | 341 |
| FSRASSIIDELFQD | 342 |
| PFLEMIHEAQQAMDI | 343 |
| PTEFIREGDDD | 344 |
| RMKDQCDKCREILSV | 345 |
| PSQAKLRRELDESLQVAERLTRKYNELLKSYQ | 346 |
| LLEQLNEQFNWVSRLANLTEGE | 347 |
| DQYYLRVTTVA | 348 |
| PSGVTEVVVKLFDS | 349 |
| PKFMETVAEKALQEYRKKHRE | 350 |

The peptides of this invention, however, are not limited to G* variants of apo J. Generally speaking G* domains from essentially any other protein preferably apo proteins are also suitable. The particular suitability of such proteins can readily be determined using assays for protective activity (e.g., protecting LDL from oxidation, and the like), e.g. as illustrated herein in the Examples. Some particularly preferred proteins include G* amphipathic helical domains or variants thereof (e.g., conservative substitutions, and the like) of proteins including, but not limited to apo AI, apo AIV, apo E, apo CII, apo CIII, and the like.

Certain preferred peptides for related to G* amphipathic helical domains related to apoproteins other than apo J are illustrated in Table 13.

TABLE 13
Peptides for use in this invention related to
G* amphipathic helical domains related to
apoproteins other than apo J.

| | SEQ |
|---|---|
| Amino Acid Sequence | ID NO |
| WDRVKDLATVYVDVLKDSGRDYVSQF | 351 |
| (Related to the 8 to 33 region of apo AI) | |
| VATVMWDYFSQLSNNAKEAVEHLQK | 352 |
| (Related to the 7 to 31 region of apo AIV) | |
| RWELALGRFWDYLRWVQTLSEQVQEEL | 353 |
| (Related to the 25 to 51 region of apo E) | |
| LSSQVTQELRALMDETMKELKELKAYKSELEEQLT | 354 |
| (Related to the 52 to 83 region of apo E) | |
| ARLSKELQAAQARLGADMEDVCGRLV | 355 |
| (Related to the 91 to 116 region of apo E) | |
| VRLASHLRKLRKRLLRDADDLQKRLA | 356 |
| (Related to the 135 to 160 region of apo E) | |
| PLVEDMQRQWAGLVEKVQA | 357 |
| (267 to 285 of apo E.27) | |
| MSTYTGIFTDQVLSVLK | 358 |
| (Related to the 60 to 76 region of apo CII) | |
| LLSFMQGYMKHATKTAKDALSS | 359 |
| (Related to the 8 to 29 region of apo CIII) | |

E) G* Peptides Derived From apo-M.

Other G* peptides that have been found to be effective in the methods of this invention include, but are not limited to G* peptides derived from apo-M.

TABLE 14
Illustrative G* peptides.

| Peptide | SEQ ID NO |
|---|---|
| Ac-Lys-Trp-Ile-Tyr-His-Leu-Thr-Glu-Gly-Ser-Thr-Asp-Leu- | 360 |
| Arg-Thr-Glu-Gly-NH₂ | |
| Ac-Lys-Trp-Phe-Tyr-His-Leu-Thr-Glu-Gly-Ser-Thr-Asp-Leu- | 361 |
| Arg-Thr-Glu-Gly-NH₂ | |
| Ac-Lys-Trp-Leu-Tyr-His-Leu-Thr-Glu-Gly-Ser-Thr-Asp-Leu- | 362 |
| Arg-Thr-Glu-Gly-NH₂ | |
| Ac-Lys-Trp-Val-Tyr-His-Leu-Thr-Glu-Gly-Ser-Thr-Asp-Leu- | 363 |
| Arg-Thr-Glu-Gly-NH₂ | |
| Ac-Lys-Tyr-Ile-Trp-His-Leu-Thr-Glu-Gly-Ser-Thr-Asp-Leu- | 364 |
| Arg-Thr-Glu-Gly-NH₂ | |
| Ac-Lys-Trp-Ile-Tyr-His-Phe-Thr-Glu-Gly-Ser-Thr-Asp-Leu- | 365 |
| Arg-Thr-Glu-Gly-NH₂ | |
| Ac-Lys-Trp-Phe-Tyr-His-Ile-Thr-Glu-Gly-Ser-Thr-Asp-Leu- | 366 |
| Arg-Thr-Glu-Gly-NH₂ | |
| Ac-Lys-Trp-Leu-Tyr-His-Val-Thr-Glu-Gly-Ser-Thr-Asp-Leu- | 367 |
| Arg-Thr-Glu-Gly-NH₂ | |
| Ac-Lys-Trp-Val-Tyr-His-Tyr-Thr-Glu-Gly-Ser-Thr-Asp-Leu- | 368 |
| Arg-Thr-Glu-Gly-NH₂ | |
| Ac-Lys-Tyr-Ile-Trp-His-Phe-Thr-Glu-Gly-Ser-Thr-Asp-Leu- | 369 |
| Arg-Thr-Glu-Gly-NH₂ | |
| Ac-Lys-Tyr-Ile-Trp-His-Ile-Thr-Glu-Gly-Ser-Thr-Asp-Leu- | 370 |
| Arg-Thr-Glu-Gly-NH₂ | |
| Ac-Lys-Tyr-Ile-Trp-His-Val-Thr-Glu-Gly-Ser-Thr-Asp-Leu- | 371 |
| Arg-Thr-Glu-Gly-NH₂ | |
| Ac-Lys-Tyr-Ile-Trp-His-Tyr-Thr-Glu-Gly-Ser-Thr-Asp-Leu- | 372 |
| Arg-Thr-Glu-Gly-NH₂ | |
| Ac-Lys-Phe-Ile-Trp-His-Leu-Thr-Glu-Gly-Ser-Thr-Asp-Leu- | 373 |
| Arg-Thr-Glu-Gly-NH₂ | |
| Ac-Lys-Leu-Ile-Trp-His-Leu-Thr-Glu-Gly-Ser-Thr-Asp-Leu- | 374 |
| Arg-Thr-Glu-Gly-NH₂ | |
| Ac-Lys-Ile-Ile-Trp-His-Leu-Thr-Glu-Gly-Ser-Thr-Asp-Leu- | 375 |
| Arg-Thr-Glu-Gly-NH₂ | |
| Ac-Lys-Tyr-Ile-Trp-Phe-Leu-Thr-Glu-Gly-Ser-Thr-Asp-Leu- | 376 |
| Arg-Thr-Glu-Gly-NH₂ | |
| Ac-Lys-Trp-Ile-Tyr-Phe-Leu-Thr-Glu-Gly-Ser-Thr-Asp-Leu- | 377 |
| Arg-Thr-Glu-Gly-NH₂ | |
| Ac-Lys-Trp-Ile-Tyr-Leu-Leu-Thr-Glu-Gly-Ser-Thr-Asp-Leu- | 378 |
| Arg-Thr-Glu-Gly-NH₂ | |
| Ac-Lys-Trp-Ile-Tyr-His-Phe-Thr-Glu-Gly-Ser-Thr-Asp-Leu- | 379 |
| Arg-Thr-Glu-Gly-NH₂ | |
| Ac-Lys-Trp-Ile-Tyr-His-Tyr-Thr-Glu-Gly-Ser-Thr-Asp-Leu- | 380 |
| Arg-Thr-Glu-Gly-NH₂ | |
| Ac-Lys-Trp-Ile-Tyr-His-Ile-Thr-Glu-Gly-Ser-Thr-Asp-Leu- | 381 |
| Arg-Thr-Glu-Gly-NH₂ | |
| Ac-Lys-Trp-Ile-Tyr-His-Leu-Ser-Glu-Gly-Ser-Thr-Asp-Leu- | 382 |
| Arg-Thr-Glu-Gly-NH₂ | |
| Ac-Lys-Trp-Ile-Tyr-His-Leu-Thr-Asp-Gly-Ser-Thr-Asp-Leu- | 383 |
| Arg-Thr-Glu-Gly-NH₂ | |
| Ac-Lys-Trp-Ile-Tyr-His-Leu-Thr-Glu-Gly-Thr-Ser-Asp-Leu- | 384 |
| Arg-Thr-Glu-Gly-NH₂ | |
| Ac-Lys-Trp-Ile-Tyr-His-Leu-Thr-Glu-Gly-Ser-Thr-Glu-Leu- | 385 |
| Arg-Thr-Glu-Gly-NH₂ | |
| Ac-Lys-Trp-Ile-Tyr-His-Leu-Thr-Glu-Gly-Ser-Thr-Asp-Phe- | 386 |
| Arg-Thr-Glu-Gly-NH₂ | |
| Ac-Lys-Trp-Ile-Tyr-His-Leu-Thr-Glu-Gly-Ser-Thr-Asp-Tyr- | 387 |
| Arg-Thr-Glu-Gly-NH₂ | |
| Ac-Lys-Trp-Ile-Tyr-His-Leu-Thr-Glu-Gly-Ser-Thr-Asp-Ile- | 388 |
| Arg-Thr-Glu-Gly-NH₂ | |
| Ac-Lys-Trp-Ile-Tyr-His-Leu-Thr-Glu-Gly-Ser-Thr-Asp-Val- | 389 |
| Arg-Thr-Glu-Gly-NH₂ | |
| Ac-Lys-Trp-Ile-Tyr-His-Leu-Thr-Glu-Gly-Ser-Thr-Asp-Leu- | 390 |
| Lys-Thr-Glu-Gly-NH₂ | |
| Ac-Lys-Trp-Ile-Tyr-His-Leu-Thr-Glu-Gly-Ser-Thr-Asp-Leu- | 391 |
| Arg-Ser-Glu-Gly-NH₂ | |
| Ac-Lys-Trp-Ile-Tyr-His-Leu-Thr-Glu-Gly-Ser-Thr-Asp-Leu- | 392 |
| Arg-Thr-Asp-Gly-NH₂ | |
| Ac-Lys-Trp-Ile-Tyr-His-Leu-Thr-Glu-Gly-Ser-Thr-Asp-Ile- | 393 |
| Lys-Thr-Glu-Gly-NH₂ | |
| Ac-Lys-Trp-Ile-Tyr-His-Leu-Thr-Glu-Gly-Ser-Thr-Asp-Ile- | 394 |
| Arg-Ser-Glu-Gly-NH₂ | |
| Ac-Lys-Trp-Ile-Tyr-His-Leu-Thr-Glu-Gly-Ser-Thr-Asp-Ile- | 395 |
| Lys-Ser-Glu-Gly-NH₂ | |
| Ac-Lys-Trp-Ile-Tyr-His-Leu-Thr-Glu-Gly-Ser-Thr-Asp-Ile- | 396 |
| Lys-Ser-Asp-Gly-NH₂ | |
| Ac-Arg-Trp-Ile-Tyr-His-Leu-Thr-Glu-Gly-Ser-Thr-Asp-Leu- | 397 |
| Arg-Thr-Glu-Gly-NH₂ | |
| Ac-Arg-Tyr-Ile-Trp-His-Leu-Thr-Glu-Gly-Ser-Thr-Asp-Ile- | 398 |
| Arg-Thr-Glu-Gly-NH₂ | |
| Ac-Arg-Trp-Ile-Tyr-His-Leu-Thr-Glu-Gly-Ser-Thr-Asp-Ile- | 399 |
| Arg-Thr-Asp-Gly-NH₂ | |
| Ac-Arg-Trp-Ile-Phe-His-Leu-Thr-Glu-Gly-Ser-Thr-Asp-Ile- | 400 |
| Arg-Thr-Glu-Gly-NH₂ | |
| Ac-Arg-Trp-Ile-Tyr-His-Leu-Thr-Glu-Gly-Ser-Thr-Asp-Leu- | 401 |
| Lys-Thr-Glu-Gly-NH₂ | |
| Ac-Arg-Trp-Ile-Tyr-His-Leu-Thr-Asp-Gly-Ser-Thr-Asp-Ile- | 402 |
| Arg-Thr-Glu-Gly-NH₂ | |
| Ac-Arg-Trp-Ile-Tyr-His-Leu-Thr-Asp-Gly-Ser-Thr-Asp-Leu- | 403 |
| Arg-Thr-Glu-Gly-NH₂ | |
| Ac-Arg-Trp-Ile-Tyr-Phe-Leu-Thr-Glu-Gly-Ser-Thr-Asp-Ile- | 404 |
| Arg-Thr-Glu-Gly-NH₂ | |
| Ac-Arg-Trp-Ile-Tyr-Phe-Leu-Thr-Glu-Gly-Ser-Thr-Asp-Leu- | 405 |
| Arg-Thr-Glu-Gly-NH₂ | |
| Ac-Lys-Trp-Phe-Tyr-His-Leu-Thr-Glu-Gly-Ser-Thr-Asp-Phe- | 406 |
| Arg-Thr-Glu-Gly-NH₂ | |
| Ac-Arg-Trp-Phe-Tyr-His-Leu-Thr-Glu-Gly-Ser-Thr-Asp-Leu- | 407 |
| Arg-Thr-Glu-Gly-NH₂ | |
| Ac-Lys-Trp-Ile-Phe-His-Leu-Thr-Glu-Gly-Ser-Thr-Asp-Ile- | 408 |
| Arg-Thr-Asp-Gly-NH₂ | |
| Ac-Arg-Trp-Ile-Tyr-His-Leu-Thr-Glu-Gly-Ser-Thr-Asp-Ile- | 409 |
| Arg-Thr-Asp-Gly-NH₂ | |
| Ac-Arg-Trp-Ile-Tyr-His-Leu-Thr-Glu-Gly-Ser-Thr-Asp-Leu- | 410 |
| Arg-Thr-Asp-Gly-NH₂ | |
| Ac-Lys-Trp-Ile-Tyr-His-Leu-Thr-Glu-Gly-Ser-Thr-Asp-Ile- | 411 |
| Lys-Thr-Glu-Gly-NH₂ | |
| Ac-Lys-Trp-Ile-Tyr-His-Leu-Thr-Glu-Gly-Ser-Thr-Asp-Ile- | 412 |
| Lys-Thr-Asp-Gly-NH₂ | |
| Ac-Lys-Trp-Ile-Tyr-His-Leu-Thr-Glu-Gly-Ser-Thr-Asp-Phe- | 413 |
| Lys-Thr-Glu-Gly-NH₂ | |
| Ac-Lys-Trp-Ile-Tyr-His-Leu-Thr-Glu-Gly-Ser-Thr-Asp-Tyr- | 414 |
| Lys-Thr-Glu-Gly-NH₂ | |
| Ac-Lys-Trp-Ile-Tyr-His-Leu-Thr-Glu-Gly-Ser-Thr-Asp-Ile- | 415 |
| Arg-Thr-Glu-Gly-NH₂ | |
| Ac-Lys-Trp-Phe-Tyr-His-Phe-Thr-Glu-Gly-Ser-Thr-Asp-Leu- | 416 |
| Arg-Thr-Glu-Gly-NH₂ | |
| Ac-Arg-Trp-Phe-Tyr-His-Phe-Thr-Glu-Gly-Ser-Thr-Asp-Leu- | 417 |
| Arg-Thr-Glu-Gly-NH₂ | |
| Ac-Lys-Trp-Phe-Tyr-His-Phe-Thr-Glu-Gly-Ser-Thr-Asp-Phe- | 418 |
| Arg-Thr-Glu-Gly-NH₂ | |
| Ac-Lys-Trp-Phe-Tyr-His-Phe-Thr-Asp-Gly-Ser-Thr-Asp-Ile- | 419 |
| Arg-Thr-Glu-Gly-NH₂ | |
| Ac-Arg-Trp-Phe-Tyr-His-Phe-Thr-Glu-Gly-Ser-Thr-Asp-Leu- | 420 |
| Arg-Thr-Glu-Gly-NH₂ | |
| Ac-Arg-Trp-Phe-Tyr-His-Phe-Thr-Glu-Gly-Ser-Thr-Asp-Phe- | 421 |
| Arg-Thr-Glu-Gly-NH₂ | |
| Ac-Arg-Trp-Phe-Tyr-His-Phe-Thr-Glu-Gly-Ser-Thr-Asp-Phe- | 422 |
| Arg-Thr-Asp-Gly-NH₂ | |
| Ac-Glu-Lys-Cys-Val-Glu-Glu-Phe-Lys-Ser-Leu-Thr-Ser-Cys- | 423 |
| Leu-Asp-Ser-Lys-Ala-Phe-NH₂ | |
| Ac-Asp-Lys-Cys-Val-Glu-Glu-Phe-Lys-Ser-Leu-Thr-Ser-Cys- | 424 |
| Leu-Asp-Ser-Lys-Ala-Phe-NH₂ | |
| Ac-Glu-Lys-Cys-Val-Asp-Glu-Phe-Lys-Ser-Leu-Thr-Ser-Cys- | 425 |
| Leu-Asp-Ser-Lys-Ala-Phe-NH₂ | |
| Ac-Glu-Lys-Cys-Val-Glu-Asp-Phe-Lys-Ser-Leu-Thr-Ser-Cys- | 426 |
| Leu-Asp-Ser-Lys-Ala-Phe-NH₂ | |
| Ac-Glu-Arg-Cys-Val-Glu-Glu-Phe-Lys-Ser-Leu-Thr-Ser-Cys- | 427 |
| Leu-Asp-Ser-Lys-Ala-Phe-NH₂ | |
| Ac-Asp-Lys-Cys-Val-Asp-Asp-Phe-Lys-Ser-Leu-Thr-Ser-Cys- | 428 |
| Leu-Asp-Ser-Lys-Ala-Phe-NH₂ | |
| Ac-Asp-Arg-Cys-Val-Glu-Glu-Phe-Lys-Ser-Leu-Thr-Ser-Cys- | 429 |
| Leu-Asp-Ser-Lys-Ala-Phe-NH₂ | |
| Ac-Glu-Arg-Cys-Val-Asp-Asp-Phe-Lys-Ser-Leu-Thr-Ser-Cys- | 430 |
| Leu-Asp-Ser-Lys-Ala-Phe-NH₂ | |
| Ac-Glu-Lys-Cys-Val-Glu-Glu-Phe-Lys-Ser-Phe-Thr-Ser-Cys- | 431 |
| Leu-Asp-Ser-Lys-Ala-Phe-NH₂ | |
| Ac-Glu-Lys-Cys-Val-Glu-Glu-Phe-Lys-Ser-Ile-Thr-Ser-Cys- | 432 |
| Leu-Asp-Ser-Lys-Ala-Phe-NH₂ | |
| Ac-Glu-Lys-Cys-Val-Glu-Glu-Phe-Lys-Ser-Val-Thr-Ser-Cys- | 433 |
| Leu-Asp-Ser-Lys-Ala-Phe-NH₂ | |
| Ac-Glu-Arg-Cys-Val-Glu-Glu-Phe-Lys-Ser-Tyr-Thr-Ser-Cys- | 434 |
| Leu-Asp-Ser-Lys-Ala-Phe-NH₂ | |
| Ac-Glu-Arg-Cys-Val-Glu-Glu-Phe-Lys-Ser-Phe-Thr-Ser-Cys- | 435 |
| Leu-Asp-Ser-Lys-Ala-Phe-NH₂ | |
| Ac-Glu-Arg-Cys-Val-Glu-Glu-Phe-Lys-Ser-Ile-Thr-Ser-Cys- | 436 |
| Leu-Asp-Ser-Lys-Ala-Phe-NH₂ | |
| Ac-Glu-Arg-Cys-Val-Glu-Glu-Phe-Lys-Ser-Val-Thr-Ser-Cys- | 437 |
| Leu-Asp-Ser-Lys-Ala-Phe-NH₂ | |
| Ac-Glu-Arg-Cys-Val-Glu-Glu-Phe-Lys-Ser-Tyr-Thr-Ser-Cys- | 438 |
| Leu-Asp-Ser-Lys-Ala-Phe-NH₂ | |
| Ac-Glu-Lys-Cys-Val-Glu-Glu-Phe-Lys-Ser-Phe-Thr-Thr-Cys- | 439 |
| Leu-Asp-Ser-Lys-Ala-Phe-NH₂ | |
| Ac-Glu-Lys-Cys-Val-Glu-Glu-Phe-Lys-Ser-Ile-Ser-Ser-Cys- | 440 |
| Leu-Asp-Ser-Lys-Ala-Phe-NH₂ | |
| Ac-Glu-Lys-Cys-Val-Glu-Glu-Phe-Lys-Ser-Val-Ser-Thr-Cys- | 441 |
| Leu-Asp-Ser-Lys-Ala-Phe-NH₂ | |
| Ac-Glu-Lys-Cys-Val-Glu-Glu-Phe-Lys-Ser-Tyr-Thr-Ser-Cys- | 442 |
| Leu-Asp-Ser-Lys-Ala-Phe-NH₂ | |
| Ac-Glu-Lys-Cys-Val-Glu-Glu-Phe-Lys-Ser-Phe-Thr-Thr-Cys- | 443 |
| Leu-Asp-Ser-Lys-Ala-Phe-NH₂ | |
| Ac-Glu-Lys-Cys-Val-Glu-Glu-Phe-Lys-Ser-Phe-Ser-Ser-Cys- | 444 |
| Leu-Asp-Ser-Lys-Ala-Phe-NH₂ | |
| Ac-Glu-Lys-Cys-Val-Glu-Glu-Phe-Lys-Ser-Phe-Thr-Ser-Cys- | 445 |
| Leu-Asp-Ser-Lys-Ala-Phe-NH₂ | |
| Ac-Glu-Lys-Cys-Val-Glu-Glu-Phe-Lys-Ser-Phe-Thr-Ser-Cys- | 446 |
| Leu-Asp-Ser-Lys-Ala-Phe-NH₂ | |
| Ac-Glu-Lys-Cys-Val-Glu-Glu-Phe-Lys-Ser-Phe-Thr-Ser-Cys- | 447 |
| Leu-Asp-Ser-Lys-Ala-Phe-NH₂ | |
| Ac-Glu-Lys-Cys-Val-Glu-Glu-Phe-Lys-Ser-Phe-Thr-Ser-Cys- | 448 |
| Phe-Asp-Ser-Lys-Ala-Phe-NH₂ | |
| Ac-Glu-Lys-Cys-Val-Glu-Glu-Phe-Lys-Ser-Phe-Thr-Ser-Cys- | 449 |
| Phe-Glu-Ser-Lys-Ala-Phe-NH₂ | |
| Ac-Glu-Lys-Cys-Val-Glu-Glu-Phe-Lys-Ser-Phe-Thr-Ser-Cys- | 450 |
| Leu-Glu-Ser-Lys-Ala-Phe-NH₂ | |
| Ac-Glu-Lys-Cys-Val-Glu-Glu-Phe-Lys-Ser-Phe-Thr-Ser-Cys- | 451 |
| Ile-Asp-Ser-Lys-Ala-Phe-NH₂ | |
| Ac-Glu-Lys-Cys-Val-Glu-Glu-Leu-Lys-Ser-Phe-Thr-Ser-Cys- | 452 |
| Phe-Asp-Ser-Lys-Ala-Phe-NH₂ | |
| Ac-Asp-Lys-Cys-Val-Glu-Glu-Phe-Lys-Ser-Phe-Thr-Ser-Cys- | 453 |
| Phe-Asp-Ser-Lys-Ala-Phe-NH₂ | |
| Ac-Asp-Lys-Cys-Val-Glu-Glu-Phe-Lys-Ser-Phe-Thr-Ser-Cys- | 454 |
| Phe-Glu-Ser-Lys-Ala-Phe-NH₂ | |
| Ac-Glu-Arg-Cys-Val-Glu-Glu-Phe-Lys-Ser-Phe-Thr-Ser-Cys- | 455 |
| Phe-Asp-Ser-Lys-Ala-Phe-NH₂ | |
| Ac-Glu-Lys-Cys-Phe-Glu-Glu-Phe-Lys-Ser-Phe-Thr-Ser-Cys- | 456 |
| Phe-Asp-Ser-Lys-Ala-Phe-NH₂ | |
| Ac-Glu-Lys-Cys-Phe-Glu-Glu-Phe-Lys-Ser-Phe-Thr-Ser-Cys- | 457 |
| Phe-Glu-Ser-Lys-Ala-Phe-NH₂ | |
| Ac-Glu-Lys-Cys-Val-Glu-Glu-Phe-Lys-Ser-Phe-Ser-Ser-Cys- | 458 |
| Phe-Glu-Ser-Lys-Ala-Phe-NH₂ | |
| Ac-Glu-Lys-Cys-Val-Glu-Glu-Phe-Lys-Ser-Phe-Gln-Ser-Cys- | 459 |
| Phe-Asp-Ser-Lys-Ala-Phe-NH₂ | |
| Ac-Glu-Lys-Cys-Phe-Glu-Glu-Phe-Lys-Ser-Phe-Gln-Ser-Cys- | 460 |
| Phe-Asp-Ser-Lys-Ala-Phe-NH₂ | |
| Ac-Glu-Lys-Cys-Val-Glu-Glu-Phe-Lys-Gln-Phe-Thr-Ser-Cys- | 461 |
| Phe-Asp-Ser-Lys-Ala-Phe-NH₂ | |
| Ac-Glu-Lys-Cys-Val-Glu-Glu-Phe-Lys-Gln-Leu-Thr-Ser-Cys- | 462 |
| Leu-Asp-Ser-Lys-Ala-Phe-NH₂ | |
| Ac-Glu-Lys-Cys-Phe-Glu-Glu-Phe-Lys-Ser-Phe-Gln-Ser-Cys- | 463 |
| Leu-Asp-Ser-Lys-Ala-Phe-NH₂ | |
| Ac-Glu-Lys-Cys-Val-Glu-Glu-Phe-Lys-Gln-Phe-Thr-Ser-Cys- | 464 |
| Phe-Asp-Ser-Lys-Ala-Phe-NH₂ | |
| Ac-Glu-Lys-Cys-Val-Glu-Glu-Phe-Lys-Ser-Phe-Thr-Ser-Cys- | 465 |
| Phe-Glu-Ser-Lys-Ala-Phe-NH₂ | |
| Ac-Glu-Arg-Cys-Phe-Glu-Glu-Phe-Lys-Ser-Phe-Thr-Ser-Cys- | 466 |
| Phe-Asp-Ser-Lys-Ala-Phe-NH₂ | |
| Ac-Asp-Lys-Cys-Phe-Glu-Glu-Phe-Lys-Ser-Phe-Thr-Ser-Cys- | 467 |
| Phe-Asp-Ser-Lys-Ala-Phe-NH₂ | |
| Ac-Glu-Arg-Cys-Val-Glu-Glu-Phe-Lys-Ser-Leu-Thr-Ser-Cys- | 468 |
| Leu-Glu-Ser-Lys-Ala-Phe-NH₂ | |
| Ac-Glu-Lys-Cys-Val-Glu-Glu-Phe-Lys-Ser-Leu-Thr-Ser-Cys- | 469 |
| Leu-Asp-Ser-Lys-Phe-Phe-NH₂ | |
| Ac-Glu-Lys-Cys-Phe-Glu-Glu-Phe-Lys-Ser-Phe-Thr-Ser-Cys- | 470 |
| Phe-Asp-Ser-Lys-Phe-Phe-NH₂ | |
| Ac-Asp-Lys-Cys-Phe-Glu-Glu-Phe-Lys-Ser-Phe-Thr-Ser-Cys- | 471 |
| Leu-Asp-Ser-Lys-Phe-Phe-NH₂ | |
| Ac-Asp-Lys-Cys-Phe-Glu-Glu-Phe-Lys-Ser-Phe-Thr-Ser-Cys- | 472 |
| Leu-Glu-Ser-Lys-Phe-Phe-NH₂ | |
| Ac-Asp-Lys-Cys-Phe-Glu-Glu-Leu-Lys-Ser-Phe-Thr-Ser-Cys- | 473 |
| Leu-Asp-Ser-Lys-Phe-Phe-NH₂ | |
| Ac-Glu-Arg-Cys-Phe-Glu-Glu-Phe-Lys-Ser-Phe-Thr-Ser-Cys- | 474 |
| Leu-Asp-Ser-Lys-Phe-Phe-NH₂ | |
| Ac-Glu-Lys-Ala-Val-Glu-Glu-Phe-Lys-Ser-Phe-Thr-Ser-Cys- | 475 |
| Leu-Asp-Ser-Lys-Ala-Phe-NH₂ | |
| Ac-Asp-Lys-Ala-Val-Glu-Glu-Phe-Lys-Ser-Phe-Thr-Ser-Cys- | 476 |
| Leu-Asp-Ser-Lys-Phe-Phe-NH₂ | |
| Ac-Glu-Lys-Ala-Val-Glu-Glu-Phe-Lys-Ser-Phe-Thr-Ser-Ala- | 477 |
| Leu-Asp-Ser-Lys-Ala-Phe-NH₂ | |
| Ac-Asp-Lys-Ala-Val-Glu-Glu-Phe-Lys-Ser-Phe-Thr-Ser-Ala- | 478 |
| Leu-Asp-Ser-Lys-Ala-Phe-NH₂ | |
| Ac-Asp-Arg-Ala-Phe-Glu-Glu-Phe-Lys-Ser-Phe-Thr-Ser-Cys- | 479 |
| Leu-Asp-Ser-Lys-Phe-Phe-NH₂ | |
| Ac-Asp-Arg-Ala-Phe-Glu-Glu-Phe-Lys-Ser-Phe-Thr-Ser-Ala- | 480 |
| Leu-Asp-Ser-Lys-Phe-Phe-NH₂ | |
| Ac-Asp-Lys-Cys-Phe-Glu-Glu-Phe-Lys-Ser-Phe-Thr-Ser-Cys- | 481 |
| Phe-Glu-Ser-Lys-Phe-Phe-NH₂ | |
| Ac-Glu-Lys-Cys-Tyr-Glu-Glu-Phe-Lys-Ser-Phe-Thr-Ser-Cys- | 482 |
| Leu-Asp-Ser-Lys-Phe-Phe-NH₂ | |
| Ac-Asp-Lys-Cys-Trp-Glu-Glu-Phe-Lys-Ser-Phe-Thr-Ser-Cys- | 483 |
| Leu-Asp-Ser-Lys-Phe-Phe-NH₂ | |
| Ac-Glu-Lys-Cys-Phe-Glu-Glu-Phe-Lys-Ser-Tyr-Thr-Ser-Cys- | 484 |
| Leu-Asp-Ser-Lys-Phe-Phe-NH₂ | |
| Ac-Glu-Lys-Cys-Phe-Glu-Glu-Phe-Lys-Ser-Trp-Thr-Ser-Cys- | 485 |
| Leu-Asp-Ser-Lys-Phe-Phe-NH₂ | |
| Ac-Glu-Lys-Cys-Val-Glu-Glu-Phe-Lys-Ser-Trp-Thr-Ser-Cys- | 486 |
| Leu-Asp-Ser-Lys-Ala-Phe-NH₂ | |
| Ac-Asp-Lys-Cys-Phe-Glu-Glu-Phe-Lys-Ser-Trp-Thr-Ser-Cys- | 487 |
| Leu-Asp-Ser-Lys-Ala-Phe-NH₂ | |

Other suitable peptides include, but are not limited to the peptides of Table 15.

TABLE 15
Illustrative peptides having an improved hydrophobic phase.

| Name | Peptide | SEQ ID NO |
|---|---|---|
| V2W3A5F1017- | Ac-Asp-Val-Trp-Lys-Ala-Ala-Tyr-Asp-Lys-Phe- | 488 |
| D-4F | Ala-Glu-Lys-Phe-Lys-Glu-Phe-Phe-NH₂ | |
| V2W3F10-D-4F | Ac-Asp-Val-Trp-Lys-Ala-Phe-Tyr-Asp-Lys-Phe- | 489 |
| | Ala-Glu-Lys-Phe-Lys-Glu-Ala-Phe-NH₂ | |
| W3-D-4F | Ac-Asp-Phe-Trp-Lys-Ala-Phe-Tyr-Asp-Lys-Val- | 490 |
| | Ala-Glu-Lys-Phe-Lys-Glu-Ala-Phe-NH₂ | |
| | Ac-Phe-Phe-Glu-Lys-Phe-Lys-Glu-Ala-Phe-Lys- | 491 |
| | Asp-Tyr-Ala-Ala-Lys-Trp-Val-Asp-NH₂ | |
| | Ac-Phe-Als-Glu-Lys-Phe-Lys-Glu-Ala-Phe-Lys- | 492 |
| | Asp-Tyr-Phe-Ala-Lys-Trp-Val-Asp-NH₂ | |
| | Ac-Phe-Ala-Glu-Lys-Phe-Lys-Glu-Ala-Val-Lys- | 493 |
| | Asp-Tyr-Phe-Ala-Lys-Trp-Phe-Asp-NH₂ | |

The peptides described here (V2W3A5F10,17-D-4F; V2W3F10-D-4F; W3-D-4F) may be more potent than the original D-4F.

Still other suitable peptides include, but are not limited to: P¹-Dimethyltyrosine-D-Arg-Phe-Lys-P² (SEQ ID NO:1) and P¹-Dimethyltyrosine-Arg-Glu-Leu-P² (SEQ ID NO:2), where P1 and P2 are protecting groups as described herein. In certain embodiments, these peptides include, but are not limited to BocDimethyltyrosine-D-Arg-Phe-Lys(OtBu) (SEQ ID NO:5) and BocDimethyltyrosine-Arg-Glu-Leu(OtBu) (SEQ ID NO:6).

In certain embodiments, the peptides of this invention include 8 peptides comprising or consisting of the amino acid sequence LAEYHAK (SEQ ID NO: 8) comprising at least one D amino acid and/or at least one or two terminal protecting groups. In certain embodiments, this invention includes a A peptide that ameliorates one or more symptoms of an inflammatory condition, wherein the peptide: ranges in length from about 3 to about 10 amino acids; comprises an amino acid sequence where the sequence comprises acidic or basic amino acids alternating with aromatic or hydrophobic amino acids; comprises hydrophobic terminal amino acids or terminal amino acids bearing a hydrophobic protecting group; is not the sequence LAEYHAK (SEQ ID NO: 8) comprising all L amino acids; where the peptide converts pro-inflammatory HDL to anti-inflammatory HDL and/or makes anti-inflammatory HDL more anti-inflammatory.

It is also noted that the peptides listed in the Tables herein are not fully inclusive. Using the teaching provided herein, other suitable peptides can routinely be produced (e.g. by conservative or semi-conservative substitutions (e.g. D replaced by E), extensions, deletions, and the like). Thus, for example, one embodiment utilizes truncations of any one or more of peptides identified by SEQ ID Nos:331-359.

Longer peptides are also suitable. Such longer peptides may entirely form a class G or G* amphipathic helix, or the G amphipathic helix (helices) can form one or more domains of the peptide. In addition, this invention contemplates multimeric versions of the peptides. Thus, for example, the peptides illustrated in the tables herein can be coupled together (directly or through a linker (e.g. a carbon linker, or one or more amino acids) with one or more intervening amino acids). Suitable linkers include, but are not limited to Proline (-Pro-), Gly₄Ser₃ (SEQ ID NO: 494), and the like. Thus, one illustrative multimeric peptide according to this invention is (D-J336)-P-(D-J336) (i.e. Ac-L-L-E-Q-L-N-E-Q-F-N-W-V-S-R-L-A-N-L-T- Q-G-E-P-L-L-E-Q-L-N-E-Q-F-N-W-V-S-R-L-A-N-L-T-Q-G-E-NH₂, SEQ ID NO: 495).

This invention also contemplates the use of “hybrid” peptides comprising a one or more G or G* amphipathic helical domains and one or more class A amphipathic helices. Suitable class A amphipathic helical peptides are described in PCT publication WO 02/15923. Thus, by way of illustration, one such “hybrid” peptide is (D-J336)-Pro-(4F) (i.e. Ac-L-L-E-Q-L-N-E-Q-F-N-W-V-S-R-L-A-N-L-T-Q-G-E-P-D-W-F-K-A-F-Y-D-K-V-A-E-K-F-K-E-A-F-NH₂, SEQ ID NO: 496), and the like.

Using the teaching provided herein, one of skill can routinely modify the illustrated amphipathic helical peptides to produce other suitable apo J variants and/or amphipathic G and/or A helical peptides of this invention. For example, routine conservative or semi-conservative substitutions (e.g., E for D) can be made of the existing amino acids. The effect of various substitutions on lipid affinity of the resulting peptide can be predicted using the computational method described by Palgunachari et al. (1996) Arteriosclerosis, Thrombosis, & Vascular Biology 16: 328-338. The peptides can be lengthened or shortened as long as the class helix structure(s) are preserved. In addition, substitutions can be made to render the resulting peptide more similar to peptide(s) endogenously produced by the subject species.

While, in preferred embodiments, the peptides of this invention utilize naturally-occurring amino acids or D forms of naturally occurring amino acids, substitutions with non-naturally occurring amino acids (e.g., methionine sulfoxide, methionine methylsulfonium, norleucine, episilon-aminocaproic acid, 4-aminobutanoic acid, tetrahydroisoquinoline-3-carboxylic acid, 8-aminocaprylic acid, 4-aminobutyric acid, Lys(N(epsilon)-trifluoroacetyl), α-aminoisobutyric acid, and the like) are also contemplated.

New peptides can be designed and/or evaluated using computational methods. Computer programs to identify and classify amphipathic helical domains are well known to those of skill in the art and many have been described by Jones et al.(1992) J. Lipid Res. 33: 287-296). Such programs include, but are not limited to the helical wheel program (WHEEL or WHEEL/SNORKEL), helical net program (HELNET, HELNET/SNORKEL, HELNET/Angle), program for addition of helical wheels (COMBO or COMBO/SNORKEL), program for addition of helical nets (COMNET, COMNET/SNORKEL, COMBO/SELECT, COMBO/NET), consensus wheel program (CONSENSUS, CONSENSUS/SNORKEL), and the like.

E) Blocking Groups and D Residues.

While the various peptides and/or amino acid pairs described herein may be be shown with no protecting groups, in certain embodiments (e.g. particularly for oral administration), they can bear one, two, three, four, or more protecting groups. The protecting groups can be coupled to the C- and/or N-terminus of the peptide(s) and/or to one or more internal residues comprising the peptide(s) (e.g., one or more R-groups on the constituent amino acids can be blocked). Thus, for example, in certain embodiments, any of the peptides described herein can bear, e.g. an acetyl group protecting the amino terminus and/or an amide group protecting the carboxyl terminus. One example of such a “dual protected peptide is Ac-L-L-E-Q-L-N-E-Q-F-N-W-V-S-R-L-A-N-L-T-Q-G-E-NH₂ (SEQ ID NO:331 with blocking groups), either or both of these protecting groups can be eliminated and/or substituted with another protecting group as described herein.

Without being bound by a particular theory, it was a discovery of this invention that blockage, particularly of the amino and/or carboxyl termini of the subject peptides of this invention greatly improves oral delivery and significantly increases serum half-life.

A wide number of protecting groups are suitable for this purpose. Such groups include, but are not limited to acetyl, amide, and alkyl groups with acetyl and alkyl groups being particularly preferred for N-terminal protection and amide groups being preferred for carboxyl terminal protection. In certain particularly preferred embodiments, the protecting groups include, but are not limited to alkyl chains as in fatty acids, propeonyl, formyl, and others. Particularly preferred carboxyl protecting groups include amides, esters, and ether-forming protecting groups. In one preferred embodiment, an acetyl group is used to protect the amino terminus and an amide group is used to protect the carboxyl terminus. These blocking groups enhance the helix-forming tendencies of the peptides. Certain particularly preferred blocking groups include alkyl groups of various lengths, e.g. groups having the formula: CH₃—(CH₂)ₙ—CO— where n ranges from about 1 to about 20, preferably from about 1 to about 16 or 18, more preferably from about 3 to about 13, and most preferably from about 3 to about 10.

In certain particularly preferred embodiments, the protecting groups include, but are not limited to alkyl chains as in fatty acids, propeonyl, formyl, and others. Particularly preferred carboxyl protecting groups include amides, esters, and ether-forming protecting groups. In one preferred embodiment, an acetyl group is used to protect the amino terminus and an amide group is used to protect the carboxyl terminus. These blocking groups enhance the helix-forming tendencies of the peptides. Certain particularly preferred blocking groups include alkyl groups of various lengths, e.g. groups having the formula: CH₃—(CH₂)ₙ—CO— where n ranges from about 3 to about 20, preferably from about 3 to about 16, more preferably from about 3 to about 13, and most preferably from about 3 to about 10.

Other protecting groups include, but are not limited to Fmoc, t-butoxycarbonyl (t-BOC), 9-fluoreneacetyl group, 1-fluorenecarboxylic group, 9-florenecarboxylic group, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts), 4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), Benzyloxy (Bz10), Benzyl (Bzl), Benzoyl (Bz), 3-nitro-2-pyridinesulphenyl (Npys), 1-(4,4-dimentyl-2,6-diaxocyclohexylidene)ethyl (Dde), 2,6-dichlorobenzyl (2,6-DiCl-Bzl), 2-chlorobenzyloxycarbonyl (2-Cl—Z), 2-bromobenzyloxycarbonyl (2-Br—Z), Benzyloxymethyl (Bom), cyclohexyloxy (cHxO),t-butoxymethyl (Bum), t-butoxy (tBuO), t-Butyl (tBu), Acetyl (Ac), and Trifluoroacetyl (TFA).

Protecting/blocking groups are well known to those of skill as are methods of coupling such groups to the appropriate residue(s) comprising the peptides of this invention (see, e.g., Greene et al., (1991) Protective Groups in Organic Synthesis, 2nd ed., John Wiley & Sons, Inc. Somerset, N.J.). In one preferred embodiment, for example, acetylation is accomplished during the synthesis when the peptide is on the resin using acetic anhydride. Amide protection can be achieved by the selection of a proper resin for the synthesis. During the synthesis of the peptides described herein in the examples, rink amide resin was used. After the completion of the synthesis, the semipermanent protecting groups on acidic bifunctional amino acids such as Asp and Glu and basic amino acid Lys, hydroxyl of Tyr are all simultaneously removed. The peptides released from such a resin using acidic treatment comes out with the n-terminal protected as acetyl and the carboxyl protected as NH₂ and with the simultaneous removal of all of the other protecting groups.

In certain particularly preferred embodiments, the peptides comprise one or more D-form (dextro rather than levo) amino acids as described herein. In certain embodiments at least two enantiomeric amino acids, more preferably at least 4 enantiomeric amino acids and most preferably at least 8 or 10 enantiomeric amino acids are “D” form amino acids. In certain embodiments every other, ore even every amino acid (e.g. every enantiomeric amino acid) of the peptides described herein is a D-form amino acid.

In certain embodiments at least 50% of the enantiomeric amino acids are “D” form, more preferably at least 80% of the enantiomeric amino acids are “D” form, and most preferably at least 90% or even all of the enantiomeric amino acids are “D” form amino acids.

F) Peptide Mimetics.

In addition to the peptides described herein, peptidomimetics are also contemplated. Peptide analogs are commonly used in the pharmaceutical industry as non-peptide drugs with properties analogous to those of the template peptide. These types of non-peptide compound are termed “peptide mimetics” or “peptidomimetics” (Fauchere (1986) Adv. Drug Res. 15: 29; Veber and Freidinger (1985) TINS p.392; and Evans et al. (1987) J. Med. Chem. 30: 1229) and are usually developed with the aid of computerized molecular modeling. Peptide mimetics that are structurally similar to therapeutically useful peptides may be used to produce an equivalent therapeutic or prophylactic effect.

Generally, peptidomimetics are structurally similar to a paradigm polypeptide (e.g. SEQ ID NO:5 shown in Table 1), but have one or more peptide linkages optionally replaced by a linkage selected from the group consisting of: —CH₂NH—, —CH₂S—, —CH₂—CH₂—, —CH═CH— (cis and trans), —COCH₂—, —CH(OH)CH₂—, —CH₂SO—, etc. by methods known in the art and further described in the following references: Spatola (1983) p. 267 in Chemistry and Biochemistry of Amino Acids, Peptides, and Proteins, B. Weinstein, eds., Marcel Dekker, New York,; Spatola (1983) Vega Data 1(3) Peptide Backbone Modifications. (general review); Morley (1980) Trends Pharm Sci pp. 463-468 (general review); Hudson et al. (1979) Int J Pept Prot Res 14:177-185 (—CH₂NH—, CH₂CH₂—); Spatola et al. (1986) Life Sci 38:1243-1249 (—CH₂—S); Hann, (1982) J Chem Soc Perkin Trans I 307-314 (—CH—CH—, cis and trans); Almquist et al. (1980) J Med Chem. 23:1392-1398 (—COCH₂—); Jennings-White et al.(1982) Tetrahedron Lett. 23:2533 (—COCH₂—); Szelke et al., European Appln. EP 45665 (1982) CA: 97:39405 (1982) (—CH(OH)CH2—); Holladay et al. (1983) Tetrahedron Lett 24:4401-4404 (—C(OH)CH₂—); and Hruby (1982) Life Sci., 31:189-199 (—CH₂—S—)).

One particularly preferred non-peptide linkage is —CH₂NH—. Such peptide mimetics may have significant advantages over polypeptide embodiments, including, for example: more economical production, greater chemical stability, enhanced pharmacological properties (half-life, absorption, potency, efficacy, etc.), reduced antigenicity, and others.

In addition, circularly permutations of the peptides described herein or constrained peptides (including cyclized peptides) comprising a consensus sequence or a substantially identical consensus sequence variation may be generated by methods known in the art (Rizo and Gierasch (1992) Ann. Rev. Biochem. 61: 387); for example, by adding internal cysteine residues capable of forming intramolecular disulfide bridges which cyclize the peptide.

G) Small Organic Molecules.

In certain embodiments, the active agents of this invention include small organic molecules, e.g. as described in copending application U.S. Ser. No. 60/600,925, filed Aug. 11, 2004. In various embodiments the small organic molecules are similar to, and in certain cases, mimetics of the tetra- and penta-peptides described in copending application U.S. Ser. No. 10/649,378, filed on Aug. 26, 2003 and U.S. Ser. No. 60/494,449, filed on August 11.

The small organic molecules of this invention typically have molecular weights less than about 900 Daltons. Typically the molecules are are highly soluble in ethyl acetate (e.g., at concentrations equal to or greater than 4 mg/mL), and also are soluble in aqueous buffer at pH 7.0.

Contacting phospholipids such as 1,2-dimyristoyl-sn-glycero-3-phosphocholine (DMPC), with the small organic molecules of this invention in an aqueous environment typically results in the formation of particles with a diameter of approximately 7.5 nm (±0.1 nm). In addition, stacked bilayers are often formed with a bilayer dimension on the order of 3.4 to 4.1 nm with spacing between the bilayers in the stack of approximately 2 nm. Vesicular structures of approximately 38 nm are also often formed. Moreover, when the molecules of this invention are administered to a mammal they render HDL more anti-inflammatory and mitigate one or more symptoms of atherosclerosis and/or other conditions characterized by an inflammatory response.

Thus, in certain embodiments, the small organic molecule is one that ameliorates one or more symptoms of a pathology characterized by an inflammatory response in a mammal (e.g. atherosclerosis), where the small molecule is soluble in in ethyl acetate at a concentration greater than 4 mg/mL, is soluble in aqueous buffer at pH 7.0, and, when contacted with a phospholipid in an aqueous environment, forms particles with a diameter of approximately 7.5 nm and forms stacked bilayers with a bilayer dimension on the order of 3.4 to 4.1 nm with spacing between the bilayers in the stack of approximately 2 nm, and has a molecular weight les than 900 daltons.

In certain embodiment, the molecule has the formula:

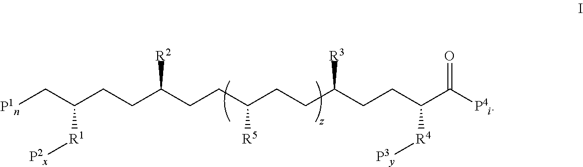

where P¹, P², P³, and P⁴ are independently selected hydrophobic protecting groups; R¹ and R⁴are independently selected amino acid R groups; n, i, x, y, and z are independently zero or 1 such that when n and x are both zero, R¹ is a hydrophobic group and when y and i are both zero, R⁴ is a hydrophobic group; R² and R³ are acidic or basic groups at pH 7.0 such that when R² is acidic, R³ is basic and when R² is basic, R³ is acidic; and R⁵, when present is selected from the group consisting of an aromatic group, an aliphatic group, a postively charged group, or a negatively charged group. In certain embodiments, R² or R³ is —(CH₂)j-COOH where j=1, 2, 3, or 4 and/or —(CH₂)j-NH₂ where j =1, 2, 3, 4, or 5, or —(CH₂)j-NH—C(═NH)—NH₂where n=1, 2, 3 or 4. In certain embodiments, R², R³, and R⁵, when present, are amino acid R groups. Thus, for example, In various embodiments R² and R³ are independently an aspartic acid R group, a glutamic acid R group, a lysine R group, a histidine R group, or an arginine R group (e.g., as illustrated in Table 1).

In certain embodiments, R¹ is selected from the group consisting of a Lys R group, a Trp R group, a Phe R group, a Leu R group, an Orn R group, pr a norLeu R group. In certain embodiments, R⁴ is selected from the group consisting of a Ser R group, a Thr R group, an Ile R group, a Leu R group, a norLeu R group, a Phe R group, or a Tyr R group.

In various embodiments x is 1, and R⁵ is an aromatic group (e.g., a Trp R group).

In various embodiments at least one of n, x, y, and i is 1 and P¹, P², P³, and P⁴ when present, are independently selected from the group consisting of polyethylene glycol (PEG), an acetyl, amide, a 3 to 20 carbon alkyl group, fmoc, 9-fluoreneacetyl group, 1-fluorenecarboxylic group, 9-fluorenecarboxylic, 9-fluorenone-1-carboxylic group, benzyloxycarbonyl, xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts),-4,4-dimethoxybenzhydryl (Mbh), Tosyl (Tos), 2,2,5,7,8-pentamethyl chroman-6-sulphonyl (Pmc), 4-methylbenzyl (MeBzl), 4-methoxybenzyl (MeOBzl), benzyloxy (BzlO), benzyl (Bzl), benzoyl (Bz), 3-nitro-2-pyridinesulphenyl (Npys), 1-(4,4-dimethyl-2,6-dioxocyclohexylidene)ethyl (Dde), 2,6-dichlorobenzyl (2,6-DiCl-Bzl), 2-chlorobenzyloxycarbonyl (2-Cl—Z), 2-bromobenzyloxycarbonyl (2-Br—Z), benzyloxymethyl (Bom), t-butoxycarbonyl (Boc), cyclohexyloxy (cHxO), t-butoxymethyl (Bum), t-butoxy (tBuO), t-Butyl (tBu), a propyl group, a butyl group, a pentyl group, a hexyl group, and trifluoroacetyl (TFA). In certain embodiments, P¹ when present and/or P² when present are independently selected from the group consisting of Boc-, Fmoc-, and Nicotinyl- and/or P³ when present and/or P⁴ when present are independently selected from the group consisting of tBu, and OtBu.

While a number of protecting groups (P¹, P², P³, P⁴) are illustrated above, this list is intended to be illustrative and not limiting. In view of the teachings provided herein, a number of other protecting/blocking groups will also be known to one of skill in the art. Such blocking groups can be selected to minimize digestion (e.g., for oral pharmaceutical delivery), and/or to increase uptake/bioavailability (e.g., through mucosal surfaces in nasal delivery, inhalation therapy, rectal administration), and/or to increase serum/plasma half-life. In certain embodiments, the protecting groups can be provided as an excipient or as a component of an excipient.

In certain embodiments, z is zero and the molecule has the formula:

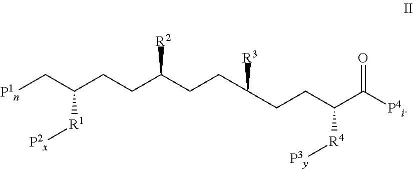

where P¹, P², P³, P⁴, R¹, R², R³, R⁴, n, x, y, and i are as described above.

In certain embodiments, z is zero and the molecule has the formula:

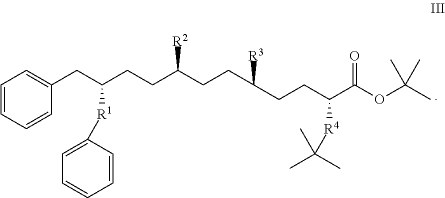

where R¹, R², R³, and R⁴ are as described above.

In one embodiment, the molecule has the formula:

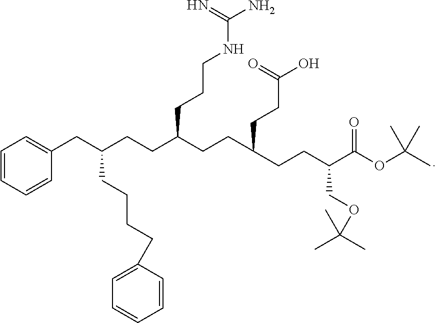

In certain embodiments, this invention contemplates small molecules having one or more of the physical and/or functional properties described herein and having the formula:

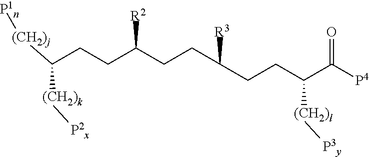

where P¹, P², P³, and P⁴ are independently selected hydrophobic protecting groups as described above, n, x, and y are independently zero or 1; j, k, and 1 are independently zero, 1, 2, 3, 4, or 5; and R² and R³ are acidic or basic groups at pH 7.0 such that when R² is acidic, R³ is basic and when R² is basic, R³ is acidic. In certain preferred embodiments, the small molecule is soluble in water; and the small molecule has a molecular weight less than about 900 Daltons. In certain embodiments, n, x, y, j, and l are 1; and k is 4.

In certain embodiments, P¹ and/or P² are aromatic protecting groups. In certain embodiments, R² and R³ are amino acid R groups, e.g., as described above. In various embodiments least one of n, x, and y, is 1 and P¹, P², P³ and P⁴ when present, are independently protecting groups, e.g. as described above selected from the group consisting of polyethylene glycol (PEG), an acetyl, amide, 3 to 20 carbon alkyl groups, Fmoc, 9-fluoreneacetyl group, 1-fluorenecarboxylic group, 9-fluorenecarboxylic, 9-fluorenone-1-carboxylic group, benzyloxycarbonyl, Xanthyl (Xan), Trityl (Trt), 4-methyltrityl (Mtt), 4-methoxytrityl (Mmt), 4-methoxy-2,3,6-trimethyl-benzenesulphonyl (Mtr), Mesitylene-2-sulphonyl (Mts),-4,4-dimethoxybenzhydryl (Mbh), Tosyl (Tos), 2,2,5,7,8-penta

### III. Functional Assays of Active Agents.

Certain active agents for use in the methods of this invention are described herein by various formulas (e.g., Formula I, above) and/or by particular sequences. In certain embodiments, preferred active agents of this invention are characterized by one or more of the following functional properties:
1. They convert pro-inflammatory HDL to anti-inflammatory HDL or make anti-inflammatory HDL more anti-inflammatory;
2. They decrease LDL-induced monocyte chemotactic activity generated by artery wall cells;
3. They stimulate the formation and cycling of pre-β HDL;
4. They raise HDL cholesterol; and/or
5. They increase HDL paraoxonase activity.

The specific agents disclosed herein, and/or agents corresponding to the various formulas described herein can readily be tested for one or more of these activities as desired.

Methods of screening for each of these functional properties are well known to those of skill in the art. In particular, it is noted that assays for monocyte chemotactic activity, HDL cholesterol, and HDL HDL paraoxonase activity are illustrated in PCT/US01/26497 (WO 2002/15923).

### IV. Peptide Preparation.

The peptides used in this invention can be chemically synthesized using standard chemical peptide synthesis techniques or, particularly where the peptide does not comprise “D” amino acid residues, can be recombinantly expressed. In certain embodiments, even peptides comprising “D” amino acid residues are recombinantly expressed. Where the polypeptides are recombinantly expressed, a host organism (e.g. bacteria, plant, fungal cells, etc.) in cultured in an environment where one or more of the amino acids is provided to the organism exclusively in a D form. Recombinantly expressed peptides in such a system then incorporate those D amino acids.

In certain preferred embodiments the peptides are chemically synthesized by any of a number of fluid or solid phase peptide synthesis techniques known to those of skill in the art. Solid phase synthesis in which the C-terminal amino acid of the sequence is attached to an insoluble support followed by sequential addition of the remaining amino acids in the sequence is a preferred method for the chemical synthesis of the polypeptides of this invention. Techniques for solid phase synthesis are well known to those of skill in the art and are described, for example, by Barany and Merrifield (1963) Solid-Phase Peptide Synthesis; pp. 3-284 in The Peptides: Analysis, Synthesis, Biology. Vol. 2: Special Methods in PeptideSynthesis, Part A.; Merrifield et al. (1963) J. Am. Chem. Soc., 85: 2149-2156, and Stewart et al. (1984) Solid Phase Peptide Synthesis, 2nd ed. Pierce Chem. Co., Rockford, Ill.

In certain embodiments, the peptides are synthesized by the solid phase peptide synthesis procedure using a benzhyderylamine resin (Beckman Bioproducts, 0.59 mmol of NH₂/g of resin) as the solid support. The COOH terminal amino acid (e.g., t-butylcarbonyl-Phe) is attached to the solid support through a 4-(oxymethyl)phenacetyl group. This is a more stable linkage than the conventional benzyl ester linkage, yet the finished peptide can still be cleaved by hydrogenation. Transfer hydrogenation using formic acid as the hydrogen donor is used for this purpose. Detailed protocols used for peptide synthesis and analysis of synthesized peptides are described in a miniprint supplement accompanying Anantharamaiah et al. (1985) J. Biol. Chem., 260(16): 10248-10255.

It is noted that in the chemical synthesis of peptides, particularly peptides comprising D amino acids, the synthesis usually produces a number of truncated peptides in addition to the desired full-length product. The purification process (e.g. HPLC) typically results in the loss of a significant amount of the full-length product.

It was a discovery of this invention that, in the synthesis of a D peptide (e.g. D-4), in order to prevent loss in purifying the longest form one can dialyze and use the mixture and thereby eliminate the last HPLC purification. Such a mixture loses about 50% of the potency of the highly purified product (e.g. per wt of protein product), but the mixture contains about 6 times more peptide and thus greater total activity.

In certain embodiments, peptided synthesis is performed utilizing a solution phase chemistry alone or in combination of with solid phase chemistries. In one approach, the final peptide is prepared by synthesizing two or more subsequences (e.g. using solid or solution phase chemistries) and then joining the subsequences in a solution phase synthesis. The solution of the 4F sequence (SEQ ID NO:13) is illustrated in the examples. To make this 18 amino acid peptide, three 6 amino acid peptides (subsequences) are first prepared. The subsequences are then coupled in solution to form the complete 4F peptide.

### V. Pharmaceutical Formulations and Devices.

A) Pharmaceutical Formulations.

In order to carry out the methods of the invention, one or more active agents of this invention are administered, e.g. to an individual diagnosed as having one or more symptoms of atherosclerosis, or as being at risk for atherosclerosis and or the various other pathologies described hereien. The active agent(s) can be administered in the “native” form or, if desired, in the form of salts, esters, amides, prodrugs, derivatives, and the like, provided the salt, ester, amide, prodrug or derivative is suitable pharmacologically, i.e., effective in the present method. Salts, esters, amides, prodrugs and other derivatives of the active agents can be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

For example, acid addition salts are prepared from the free base using conventional methodology that typically involves reaction with a suitable acid. Generally, the base form of the drug is dissolved in a polar organic solvent such as methanol or ethanol and the acid is added thereto. The resulting salt either precipitates or can be brought out of solution by addition of a less polar solvent. Suitable acids for preparing acid addition salts include both organic acids, e.g., acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluenesulfonic acid, salicylic acid, and the like, as well as inorganic acids, e.g., hydrochloric acid, hydrobromic acid, sulfuric acid, nitric acid, phosphoric acid, and the like. An acid addition salt may be reconverted to the free base by treatment with a suitable base. Particularly preferred acid addition salts of the active agents herein are halide salts, such as may be prepared using hydrochloric or hydrobromic acids. Conversely, preparation of basic salts of the active agents of this inventioni are prepared in a similar manner using a pharmaceutically acceptable base such as sodium hydroxide, potassium hydroxide, ammonium hydroxide, calcium hydroxide, trimethylamine, or the like. Particularly preferred basic salts include alkali metal salts, e.g., the sodium salt, and copper salts.

Preparation of esters typically involves functionalization of hydroxyl and/or carboxyl groups which may be present within the molecular structure of the drug. The esters are typically acyl-substituted derivatives of free alcohol groups, i.e., moieties that are derived from carboxylic acids of the formula RCOOH where R is alky, and preferably is lower alkyl. Esters can be reconverted to the free acids, if desired, by using conventional hydrogenolysis or hydrolysis procedures.

Amides and prodrugs can also be prepared using techniques known to those skilled in the art or described in the pertinent literature. For example, amides may be prepared from esters, using suitable amine reactants, or they may be prepared from an anhydride or an acid chloride by reaction with ammonia or a lower alkyl amine. Prodrugs are typically prepared by covalent attachment of a moiety that results in a compound that is therapeutically inactive until modified by an individual's metabolic system.

The active agents identified herein are useful for parenteral, topical, oral, nasal (or otherwise inhaled), rectal, or local administration, such as by aerosol or transdermally, for prophylactic and/or therapeutic treatment of one or more of the pathologies/indications described herein (e.g., atherosclerosis and/or symptoms thereof). The pharmaceutical compositions can be administered in a variety of unit dosage forms depending upon the method of administration. Suitable unit dosage forms, include, but are not limited to powders, tablets, pills, capsules, lozenges, suppositories, patches, nasal sprays, injectibles, implantable sustained-release formulations, lipid complexes, etc.

The active agents of this invention are typically combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, protection and uptake enhancers such as lipids, compositions that reduce the clearance or hydrolysis of the active agents, or excipients or other stabilizers and/or buffers.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s).

The excipients are preferably sterile and generally free of undesirable matter. These compositions may be sterilized by conventional, well-known sterilization techniques.

In therapeutic applications, the compositions of this invention are administered to a patient suffering from one or more symptoms of the one or more pathologies described herein, or at risk for one or more of the pathologies described herein in an amount sufficient to prevent and/or cure and/or or at least partially prevent or arrest the disease and/or its complications. An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the active agents of the formulations of this invention to effectively treat (ameliorate one or more symptoms) the patient.

The concentration of active agent(s) can vary widely, and will be selected primarily based on fluid volumes, viscosities, body weight and the like in accordance with the particular mode of administration selected and the patient's needs. Concentrations, however, will typically be selected to provide dosages ranging from about 0.1 or 1 mg/kg/day to about 50 mg/kg/day and sometimes higher. Typical dosages range from about 3 mg/kg/day to about 3.5 mg/kg/day, preferably from about 3.5 mg/kg/day to about 7.2 mg/kg/day, more preferably from about 7.2 mg/kg/day to about 11.0 mg/kg/day, and most preferably from about 11.0 mg/kg/day to about 15.0 mg/kg/day. In certain preferred embodiments, dosages range from about 10 mg/kg/day to about 50 mg/kg/day. In certain embodiments, dosages range from about 20 mg to about 50 mg given orally twice daily. It will be appreciated that such dosages may be varied to optimize a therapeutic regimen in a particular subject or group of subjects.

In certain preferred embodiments, the active agents of this invention are administered orally (e.g. via a tablet) or as an injectable in accordance with standard methods well known to those of skill in the art. In other preferred embodiments, the peptides, may also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

Other preferred formulations for topical drug delivery include, but are not limited to, ointments and creams. Ointments are semisolid preparations which are typically based on petrolatum or other petroleum derivatives. Creams containing the selected active agent are typically viscous liquid or semisolid emulsions, often either oil-in-water or water-in-oil. Cream bases are typically water-washable, and contain an oil phase, an emulsifier and an aqueous phase. The oil phase, also sometimes called the “internal” phase, is generally comprised of petrolatum and a fatty alcohol such as cetyl or stearyl alcohol; the aqueous phase usually, although not necessarily, exceeds the oil phase in volume, and generally contains a humectant. The emulsifier in a cream formulation is generally a nonionic, anionic, cationic or amphoteric surfactant. The specific ointment or cream base to be used, as will be appreciated by those skilled in the art, is one that will provide for optimum drug delivery. As with other carriers or vehicles, an ointment base should be inert, stable, nonirritating and nonsensitizing.

Unlike typical peptide formulations, the peptides of this invention comprising D-form amino acids can be administered, even orally, without protection against proteolysis by stomach acid, etc. Nevertheless, in certain embodiments, peptide delivery can be enhanced by the use of protective excipients. This is typically accomplished either by complexing the polypeptide with a composition to render it resistant to acidic and enzymatic hydrolysis or by packaging the polypeptide in an appropriately resistant carrier such as a liposome. Means of protecting polypeptides for oral delivery are well known in the art (see, e.g., U.S. Pat. No. 5,391,377 describing lipid compositions for oral delivery of therapeutic agents).

Elevated serum half-life can be maintained by the use of sustained-release protein “packaging” systems. Such sustained release systems are well known to those of skill in the art. In one preferred embodiment, the ProLease biodegradable microsphere delivery system for proteins and peptides (Tracy (1998) Biotechnol. Prog. 14: 108; Johnson et al. (1996), Nature Med. 2: 795; Herbert et al. (1998), Pharmaceut. Res. 15, 357) a dry powder composed of biodegradable polymeric microspheres containing the active agent in a polymer matrix that can be compounded as a dry formulation with or without other agents.

The ProLease microsphere fabrication process was specifically designed to achieve a high encapsulation efficiency while maintaining integrity of the active agent. The process consists of (i) preparation of freeze-dried drug particles from bulk by spray freeze-drying the drug solution with stabilizing excipients, (ii) preparation of a drug-polymer suspension followed by sonication or homogenization to reduce the drug particle size, (iii) production of frozen drug-polymer microspheres by atomization into liquid nitrogen, (iv) extraction of the polymer solvent with ethanol, and (v) filtration and vacuum drying to produce the final dry-powder product. The resulting powder contains the solid form of the active agents, which is homogeneously and rigidly dispersed within porous polymer particles. The polymer most commonly used in the process, poly(lactide-co-glycolide) (PLG), is both biocompatible and biodegradable.

Encapsulation can be achieved at low temperatures (e.g., −40° C.). During encapsulation, the protein is maintained in the solid state in the absence of water, thus minimizing water-induced conformational mobility of the protein, preventing protein degradation reactions that include water as a reactant, and avoiding organic-aqueous interfaces where proteins may undergo denaturation. A preferred process uses solvents in which most proteins are insoluble, thus yielding high encapsulation efficiencies (e.g., greater than 95%).

In another embodiment, one or more components of the solution can be provided as a “concentrate”, e.g., in a storage container (e.g., in a premeasured volume) ready for dilution, or in a soluble capsule ready for addition to a volume of water.

The foregoing formulations and administration methods are intended to be illustrative and not limiting. It will be appreciated that, using the teaching provided herein, other suitable formulations and modes of administration can be readily devised.

B) Lipid-Based Formulations.

In certain embodiments, the active agents of this invention are administered in conjunction with one or more lipids. The lipids can be formulated as an excipient to protect and/or enhance transport/uptake of the active agents or they can be administered separately.

Without being bound by a particular theory, it was discovered of this invention that administration (e.g. oral administration) of certain phospholipids can significantly increase HDL/LDL ratios. In addition, it is believed that certain medium-length phospholipids are transported by a process different than that involved in general lipid transport. Thus, co-administration of certain medium-length phospholipids with the active agents of this invention confer a number of advantages: They protect the active agents from digestion or hydrolysis, they improve uptake, and they improve HDL/LDL ratios.

The lipids can be formed into liposomes that encapsulate the active agents of this invention and/or they can be complexed/admixed with the active agents and/or they can be covalently coupled to the active agents. Methods of making liposomes and encapsulating reagents are well known to those of skill in the art (see, e.g., Martin and Papahadjopoulos (1982) J. Biol. Chem., 257: 286-288; Papahadjopoulos et al. (1991) Proc. Natl. Acad. Sci. USA, 88: 11460-11464; Huang et al. (1992) Cancer Res., 52:6774-6781; Lasic et al. (1992) FEES Lett., 312: 255-258., and the like).

Preferred phospholipids for use in these methods have fatty acids ranging from about 4 carbons to about 24 carbons in the sn-1 and sn-2 positions. In certain preferred embodiments, the fatty acids are saturated. In other preferred embodiments, the fatty acids can be unsaturated. Various preferred fatty acids are illustrated in Table 16.

TABLE 16
Preferred fatty acids in the sn-1 and/or sn-2 position of the preferred
phospholipids for administration of active agents described herein.

| Carbon No. | Common Name | IUPAC Name |
|---|---|---|
| 3:0 | Propionoyl | Trianoic |
| 4:0 | Butanoyl | Tetranoic |
| 5:0 | Pentanoyl | Pentanoic |
| 6:0 | Caproyl | Hexanoic |
| 7:0 | Heptanoyl | Heptanoic |
| 8:0 | Capryloyl | Octanoic |
| 9:0 | Nonanoyl | Nonanoic |
| 10:0 | Capryl | Decanoic |
| 11:0 | Undcanoyl | Undecanoic |
| 12:0 | Lauroyl | Dodecanoic |
| 13:0 | Tridecanoyl | Tridecanoic |
| 14:0 | Myristoyl | Tetradecanoic |
| 15:0 | Pentadecanoyl | Pentadecanoic |
| 16:0 | Palmitoyl | Hexadecanoic |
| 17:0 | Heptadecanoyl | Heptadecanoic |
| 18:0 | Stearoyl | Octadecanoic |
| 19:0 | Nonadecanoyl | Nonadecanoic |
| 20:0 | Arachidoyl | Eicosanoic |
| 21:0 | Heniecosanoyl | Heniecosanoic |
| 22:0 | Behenoyl | Docosanoic |
| 23:0 | Trucisanoyl | Trocosanoic |
| 24:0 | Lignoceroyl | Tetracosanoic |
| 14:1 | Myristoleoyl (9-cis) | |
| 14:1 | Myristelaidoyl (9-trans) | |
| 16:1 | Palmitoleoyl (9-cis) | |
| 16:1 | Palmitelaidoyl (9-trans) | |

The fatty acids in these positions can be the same or different. Particularly preferred phospholipids have phosphorylcholine at the sn-3 position.

### VI. Administration.

Typically the active agent(s) will be administered to a mammal (e.g,. a human) in need thereof. Such a mammal will typically include a mammal (e.g. a human) having or at risk for one or more of the pathologies described herein.

The active agent(s) can be administered, as described herein, according to any of a number of standard methods including, but not limited to injection, suppository, nasal spray, time-release implant, transdermal patch, and the like. In one particularly preferred embodiment, the peptide(s) are administered orally (e.g. as a syrup, capsule, or tablet).

The methods involve the administration of a single active agent of this invention or the administration of two or more different active agents. The active agents can be provided as monomers (e.g., in separate or combined formulations), or in dimeric, oligomeric or polymeric forms. In certain embodiments, the multimeric forms may comprise associated monomers (e.g., ionically or hydrophobically linked) while certain other multimeric forms comprise covalently linked monomers (directly linked or through a linker).

While the invention is described with respect to use in humans, it is also suitable for animal, e.g. veterinary use. Thus certain preferred organisms include, but are not limited to humans, non-human primates, canines, equines, felines, porcines, ungulates, largomorphs, and the like.

The methods of this invention are not limited to humans or non-human animals showing one or more symptom(s) of the pathologies described herein, but are also useful in a prophylactic context. Thus, the active agents of this invention can be administered to organisms to prevent the onset/development of one or more symptoms of the pathologies described herein (e.g., atherosclerosis, stroke, etc.). Particularly preferred subjects in this context are subjects showing one or more risk factors for for the pathology. Thus, for example, in the case of atherosclerosis risk factors include family history, hypertension, obesity, high alcohol consumption, smoking, high blood cholesterol, high blood triglycerides, elevated blood LDL, VLDL, IDL, or low HDL, diabetes, or a family history of diabetes, high blood lipids, heart attack, angina or stroke, etc.

### VII. Drug-Eluting Stents.

Restenosis, the reclosure of a previously stenosed and subsequently dilated peripheral or coronary vessel occurs at a significant rate (e.g., 20-50% for these procedures) and is dependent on a number of clinical and morphological variables. Restenosis may begin shortly following an angioplasty procedure, but usually ceases at the end of approximately six (6) months.

A recent technology that has been developed to address the problem of restenosis is intravascular stents. Stents are typically devices that are permanently implanted (expanded) in coronary and peripheral vessels. The goal of these stents is to provide a long-term “scaffolding” or support for the diseased (stenosed) vessels. The theory being, if the vessel is supported from the inside, it will not close down or restenose.

Known stent designs include, but are not limited to monofilament wire coil stents (see, e.g., U.S. Pat. No. 4,969,458); welded metal cages (see, e.g., U.S. Pat. Nos. 4,733,665 and 4,776,337), thin-walled metal cylinders with axial slots formed around the circumference (see, e.g., U.S. Pat. Nos. 4,733,665, 4,739,762, 4,776,337, and the like). Known construction materials for use in stents include, but are not limited to polymers, organic fabrics and biocompatible metals, such as, stainless steel, gold, silver, tantalum, titanium, and shape memory alloys such as Nitinol.

To further prevent restenosis, stents can be covered and/or impregnated with one or more pharmaceutical, e.g., in controlled release formulations to inhibit cell proliferation associated with resttenosis. Most commonly such “drug-eluting” stents are designed to deliver various cancer drugs (cytotoxins).

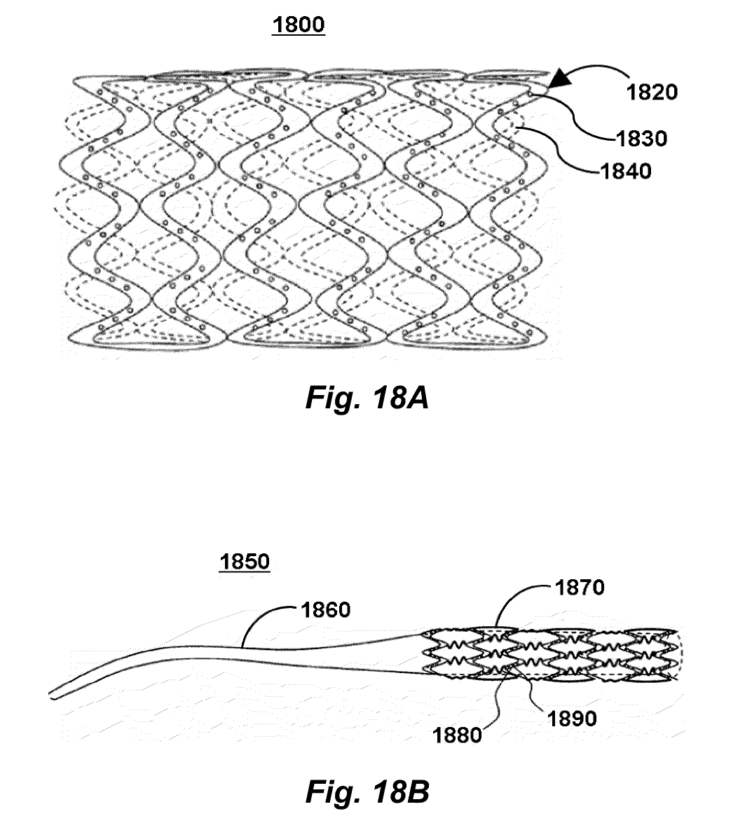

However, because of their activity in mitigating inflammatory responses, reducing and/or eliminated oxidized lipids and/or other oxidized species, inhibiting macrophage chemotactic activity and the like, the active agents described herein are well suited to prevent restenosis. Thus, in certain embodiments, this invention contemplates stents having one or more of the active agents described herein coated on the surface and/or retained within cavities or microcavities in the surface of the stent (see, e.g., FIGS. 18A and 18B).

In certain embodiments the active agents are contained within biocompatible matrices (e.g. biocompatible polymers such as urethane, silicone, and the like). Suitable biocompatible materials are described, for example, in U.S. Patent Publications 20050084515, 200500791991, 20050070996, and the like. In various embodiments the polymers include, but are not limited to silicone-urethane copolymer, a polyurethane, a phenoxy, ethylene vinyl acetate, polycaprolactone, poly(lactide-co-glycolide), polylactide, polysulfone, elastin, fibrin, collagen, chondroitin sulfate, a biocompatible polymer, a biostable polymer, a biodegradable polymer

Thus, in certain embodiments this invention provides a stent for delivering drugs to a vessel in a body. The stent typically comprises stent framework including a plurality of reservoirs formed therein. The reservoirs typically include an active agent and/or active agent-contaiing polymer positioned in the reservoir and/or coated on the surface of the stent. In various embodiments the stent is a metallic base or a polymeric base. Certain preferred stent materials include, but are not limited to stainless steel, nitinol, tantalum, MP35N alloy, platinum, titanium, a suitable biocompatible alloy, a suitable biocompatible polymer, and/or a combination thereof.

In various embodiments where the stent comprises pores (e.g. reservoirs), the pores can include micropores (e.g., having a diameter that ranges from about 10 to about 50 μm, preferably about 20 μm or less). In various embodiments the microporse have a depth in the range of about 10 μm to about 50 μm. In various embodiments the micropores extend through the stent framework having an opening on an interior surface of the stent and an opening on an exterior surface of the stent. In certain embodiments the stent can, optionally comprise a cap layer disposed on the interior surface of the stent framework, the cap layer covering at least a portion of the through-holes and providing a barrier characteristic to control an elution rate of the active agent(s) in the polymer from the interior surface of the stent framework. In various embodiments the reservoirs comprise channels along an exterior surface of the stent framework. The stent can optionally have multiple layers of polymer where different layers of polymer carry different active agent(s) and/or other drugs.

In certain embodiments the stent of optionally comprises: an adhesion layer positioned between the stent framework and the polymer. Suitable adhesion layers include, but are not limited to a polyurethane, a phenoxy, poly(lactide-co-glycolide)-, polylactide, polysulfone, polycaprolactone, an adhesion promoter, and/or a combination thereof.

In addition to stents, the active agents can be coated on or contained within essentially any implantable medical device configured for implantation in a extravascular and/or intravascular location.

Also provided are methods of manufacturing a drug-polymer stent, comprising. The methods involve providing a stent framework; cutting a plurality of reservoirs in the stent framework, e.g., using a high power laser; applying one or more of the active agents and/or a drug polymer to at least one reservoir; drying the drug polymer; applying a polymer layer to the dried drug polymer; and drying the polymer layer. The active agent(s) and/or polymer(s) can be applied by any convenient method including, but not limited to spraying, dipping, painting, brushing and dispensing.

Also provided are methods of treating a vascular condition and/or a condition characterized by an inflammatory response and/or a condition characterized by the formation of oxidized reactive species. The methods typically involve positioning a stent or other implantable device as described above within the body (e.g. within a vessel of a body) and eluting at least active agent from at least one surface of the implant.

### VIII. Enhancing Peptide Uptake.

It was also a surprising discovery of this invention that when an all L amino acid peptide (e.g. otherwise having the sequence of the peptides of this invention) is administered in conjunction with the D-form (i.e. a peptide of this invention) the uptake of the D-form peptide is increased. Thus, in certain embodiments, this invention contemplates the use of combinations of D-form and L-form peptides in the methods of this invention. The D-form peptide and the L-form peptide can have different amino acid sequences, however, in preferred embodiments, they both have amino acid sequences of peptides described herein, and in still more preferred embodiments, they have the same amino acid sequence.

It was also a discovery of this invention that concatamers of the amphipathic helix peptides of this invention are also effective in mitigating one or more symptoms of atherosclerosis. The monomers comprising the concatamers can be coupled directly together or joined by a linker. In certain embodiments, the linker is an amino acid linker (e.g. a proline), or a peptide linker (e.g. Gly₄Ser₃, SEQ ID NO:497). In certain embodiments, the concatamer is a 2 mer, more preferably a 3 mer, still more preferably a 4 mer, and most preferably 5 mer, 8 mer or 10 mer. As indicated above, the concatamer can comprise a G* related amphipathic helix as described herein combined with an apo A-I variant as described in PCT publication WO 2002/15923.

### IX. Additional Pharmacologically Active Agents.

Additional pharmacologically active agents may be delivered along with the primary active agents, e.g., the peptides of this invention. In one embodiment, such agents include, but are not limited to agents that reduce the risk of atherosclerotic events and/or complications thereof. Such agents include, but are not limited to beta blockers, beta blockers and thiazide diuretic combinations, statins, aspirin, ace inhibitors, ace receptor inhibitors (ARBs), and the like.

Suitable beta blockers include, but are not limited to cardioselective (selective beta 1 blockers), e.g., acebutolol (Sectral™), atenolol (Tenormin™), betaxolol (Kerlone™), bisoprolol (Zebeta™), metoprolol (Lopressor™), and the like. Suitable non-selective blockers (block beta 1 and beta 2 equally) include, but are not limited to carteolol (Cartrol™), nadolol (Corgard™), penbutolol (Levatol™), pindolol (Visken™), propranolol (Inderal™), timolol (Blockadren™), labetalol (Normodyne™, Trandate™), and the like.

Suitable beta blocker thiazide diuretic combinations include, but are not limited to Lopressor HCT, ZIAC, Tenoretic, Corzide, Timolide, Inderal LA 40/25, Inderide, Normozide, and the like.

Suitable statins include, but are not limited to pravastatin (Pravachol/Bristol-Myers Squibb), simvastatin (Zocor/Merck), lovastatin (Mevacor/Merck), and the like.

Suitable ace inhibitors include, but are not limited to captopril (e.g. Capoten™ by Squibb), benazepril (e.g., Lotensin™ by Novartis), enalapril (e.g., Vasotec™ by Merck), fosinopril (e.g., Monopril™ by Bristol-Myers), lisinopril (e.g. Prinivil™ by Merck or Zestril™ by Astra-Zeneca), quinapril (e.g. Accupril™ by Parke-Davis), ramipril (e.g., Altace™ by Hoechst Marion Roussel, King Pharmaceuticals), imidapril, perindopril erbumine (e.g., Aceon™ by Rhone-Polenc Rorer), trandolapril (e.g., Mavik™ by Knoll Pharmaceutical), and the like. Suitable ARBS (Ace Receptor Blockers) include but are not limited to losartan (e.g. Cozaar™ by Merck), irbesartan (e.g., Avapro™ by Sanofi), candesartan (e.g., Atacand™ by Astra Merck), valsartan (e.g., Diovan™ by Novartis), and the like.

### X. Kits for the Amelioration of One or More Symptoms of Atherosclerosis.

In another embodiment this invention provides kits for amelioration of one or more symptoms of atherosclerosis or for the prophylactic treatment of a subject (human or animal) at risk for atherosclerosis or for the treatment or prophylaxis of one or more of the other conditions described herein. The kits preferably comprise a container containing one or more of the active agents of this invention. The active agent(s) can be provided in a unit dosage formulation (e.g. suppository, tablet, caplet, patch, etc.) and/or may be optionally combined with one or more pharmaceutically acceptable excipients.

The kit can, optionally, further comprise one or more other agents used in the treatment of heart disease and/or atherosclerosis. Such agents include, but are not limited to, beta blockers, vasodilators, aspirin, statins, ace inhibitors or ace receptor inhibitors (ARBs) and the like, e.g. as described above.

In addition, the kits optionally include labeling and/or instructional materials providing directions (i.e., protocols) for the practice of the methods or use of the “therapeutics” or “prophylactics” of this invention. Preferred instructional materials describe the use of one or more polypeptides of this invention to mitigate one or more symptoms of atherosclerosis and/or to prevent the onset or increase of one or more of such symptoms in an individual at risk for atherosclerosis and/or to mitigate one or more symptoms of a pathology characterized by an inflammatory response. The instructional materials may also, optionally, teach preferred dosages/therapeutic regiment, counter indications and the like.

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

### EXAMPLES

The following examples are offered to illustrate, but not to limit the claimed invention.

### Example 1

### Use of ApoJ-Related Peptides to Mediate Symptoms of Atherosclerosis

A) Prevention of LDL-induced monocyte chemotactic activity

FIG. 1 illustrates a comparison of the effect of D-4F (Circulation 2002;105:290-292) with the effect of an apoJ peptide made from D amino acids (D-J336, Ac-L-L-E-Q-L-N-E-Q-F-N-W-V-S-R-L-A-N-L-T-Q-G-E-NH₂, SEQ ID NO:13) on the prevention of LDL-induced monocyte chemotactic activity in vitro in a co-incubation. Human aortic endothelial cells were incubated with medium alone (no addition), with control human LDL (200 μg protein/ml) or control human LDL+control human HDL (350 μg HDL protein/ml). D-J336 or D-4F was added to other wells in a concentration range as indicated plus control human LDL (200 μg protein/ml). Following overnight incubation, the supernatants were assayed for monocyte chemotactic activity. As shown in FIG. 1, the in vitro concentration of the apoJ variant peptide that prevents LDL-induced monocyte chemotactic activity by human artery wall cells is 10 to 25 times less than the concentration required for the D-4F peptide.

B) Prevention of LDL-Induced Monocyte Chemotactic Activity by Pre-Treatment of Artery Wall Cells with D-J336

FIG. 2 illustrates a comparison of the effect of D-4F with the effect of D-J336 on the prevention of LDL induced monocyte chemotactic activity in a pre-incubation. Human aortic endothelial cells were pre-incubated with D-J336 or D-4F at 4, 2, and 1 μg/ml for DJ336 or 100, 50, 25, and 12.5 μg/ml for D-4F for 6 hrs. The cultures were then washed and were incubated with medium alone (no addition), or with control human LDL (200 μg protein/ml), or with control human LDL+control human HDL (350 μg HDL protein/ml) as assay controls. The wells that were pre-treated with peptides received the control human LDL at 200 μg protein/ml. Following overnight incubation, the supernatants were assayed for monocyte chemotactic activity.

As illustrated in FIG. 2, the ApoJ variant peptide was 10-25 times more potent in preventing LDL oxidation by artery wall cells in vitro.

### C) The Effect of apo J Peptide Mimetics on HDL Protective Capacity in LDL Receptor Null Mice.

D-4F designated as F, or the apoJ peptide made from D amino acids (D-J336, designated as J) was added to the drinking water of LDL receptor null mice (4 per group) at 0.25 or 0.5 mg per ml of drinking water. After 24- or 48-hrs blood was collected from the mice and their HDL was isolated and tested for its ability to protect against LDL-induced monocyte chemotactic activity. Assay controls included culture wells that received no lipoproteins (no addition), or control human LDL alone (designated as LDL, 200 μg cholesterol/ml), or control LDL+control human HDL (designated as +HDL, 350 μg HDL cholesterol). For testing the mouse HDL, the control LDL was added together with mouse HDL (+F HDL or +J HDL) to artery wall cell cultures. The mouse HDL was added at 100 μg cholesterol/ml respectively. After treatment with either D-4F or D-J336 the mouse HDL at 100 μg/ml was as active as 350 μg/ml of control human HDL in preventing the control LDL from inducing the artery wall cells to produce monocyte chemotactic activity. The reason for the discrepancy between the relative doses required for the D-J336 peptide relative to D-4F in vitro and in vivo may be related to the solubility of the peptides in water and we believe that when measures are taken to achieve equal solubility the D-J peptides will be much more active in vivo as they are in vitro.

### D) Protection Against LDL-Induced Monocyte Chemotactic Activity by HDL From apo E Null Mice Given Oral Peptides.

FIG. 4 illustrates the effect of oral apoA-1 peptide mimetic and apoJ peptide on HDL protective capacity. ApoE null mice (4 per group) were provided with D-4F (designated as F) at 50, 30, 20, 10, 5 μg per ml of drinking water or apoJ peptide (designated as J) at 50, 30 or 20 μg per ml of drinking water. After 24 hrs blood was collected, plasma fractionated by FPLC and fractions containing LDL (designated as mLDL for murine LDL) and fractions containing HDL (designated as mHDL) were separately pooled and HDL protective capacity against LDL oxidation as determined by LDL-induced monocyte chemotactic activity was determined. For the assay controls the culture wells received no lipoproteins (no additions), mLDL alone (at 200 μg cholesterol/ml), or mLDL+standard normal human HDL (designated as Cont. h HDL, at 350 μg HDL cholesterol/ml).

For testing the murine HDL, mLDL together with murine HDL (+F mHDL or +J mHDL) were added to artery wall cell cultures. The HDL from the mice that did not receive any peptide in their drinking water is designated as no peptide mHDL. The murine HDL was used at 100 μg cholesterol/ml. After receiving D-4F or D-J336 the murine HDL at 100 μg/ml was as active as 350 μg/ml of normal human HDL. As shown in FIG. 4, when added to the drinking water the D-J peptide was as potent as D-4F in enhancing HDL protective capacity in apo E null mice.

E) Ability of LDL Obtained from apoE Null Mice Given Oral Peptides to Induce Monocyte Chemotactic Activity.

FIG. 5 illustrates the effect of oral apo A-1 peptide mimetic and apoJ peptide on LDL susceptibility to oxidation. ApoE null mice (4 per group) were provided, in their drinking water, with D-4F (designated as F) at 50, 30, 20, 10, 5 μg per ml of drinking water or the apoJ peptide (D-J336 made from D amino acids and designated as J) at 50, 30 or 20 μg per ml of drinking water. After 24 hrs blood was collected from the mice shown in FIG. 4, plasma fractionated by FPLC and fractions containing LDL (designated as mLDL for murine LDL) were pooled and LDL susceptibility to oxidation as determined by induction of monocyte chemotactic activity was determined. For the assay controls the culture wells received no lipoproteins (no additions), mLDL alone (at 200 μg cholesterol/ml), or mLDL+standard normal human HDL (designated as Cont. h HDL, 350 μg HDL cholesterol).

Murine LDL, mLDL, from mice that received the D-4F (F mLDL) or those that received the apoJ peptide (J mLDL) were added to artery wall cell cultures. LDL from mice that did not receive any peptide in their drinking water is designated as No peptide LDL.

As shown in FIG. 5, when added to the drinking water, D-J336 was slightly more potent than D-4F in rendering the LDL from apo E null mice resistant to oxidation by human artery wall cells as determined by the induction of monocyte chemotactic activity.

F) Protection Against Phospholipid Oxidation and Induction of Monocyte Chemotactic Activity by HDL Obtained from apo E Null Mice Given Oral Peptides.

FIG. 6 illustrates the effect of oral apoA-1 peptide mimetic and apoJ peptide on HDL protective capacity. ApoE null mice (4 per group) were provided with D-4F (designated as F) at 50, 30, 20, 10, 5 μg per ml of drinking water or apoJ peptide (D-J336 made from D amino acids and designated as J) at 50, 30 or 20 μg per ml of drinking water. After 24 hrs blood was collected, plasma fractionated by FPLC and fractions containing HDL (designated as mHDL) were pooled and HDL protective capacity against PAPC oxidation as determined by the induction of monocyte chemotactic activity was determined. For the assay controls the culture wells received no lipoproteins (no additions), the phospholipid PAPC at 20 μg /ml+HPODE, at 1.0 μg/ml, or PAPC+HPODE plus standard normal human HDL (at 350 μg HDL cholesterol/ml and designated as +Cont. h HDL).

For testing the murine HDL, PAPC+HPODE together with murine HDL (+F mHDL or +J mHDL) were added to artery wall cell cultures. The HDL from mice that did not receive any peptide in their drinking water is designated as “no peptide mHDL”. The murine HDL was used at 100 μg cholesterol/ml.

The data show in FIG. 6 indicate that, when added to the drinking water, D-J336 was as potent as D-4F in causing HDL to inhibit the oxidation of a phospholipid PAPC by the oxidant HPODE in a human artery wall co-culture as measured by the generation of monocyte chemotactic activity

G) Effect of Oral apoA-1 Peptide Mimetic and apoJ Peptide on Plasma Paraoxonase Activity in Mice.

FIG. 7 shows the effect of oral apoA-1 peptide mimetic and apoJ peptide on plasma paraoxonase activity in mice. ApoE null mice (4 per group) were provided with D-4F designated as F at 50, 10, 5 or 0 μg per ml of drinking water or apoJ peptide (D-J336 made from D amino acids and designated as J) at 50, 10 or 5 μg per ml of drinking water. After 24 hrs blood was collected and plasma was assayed for PON1 activity. These data demonstrate that, when added to the drinking water, D-J336 was at least as potent as D-4F in increasing the paraoxonase activity of apo E null mice.

### Example 2

### Oral G* Peptides Increase HDL Protective Capacity In Apo E Deficient Mice

Female, 4 month old apoE deficient mice (n=4 per group) were treated with G* peptides having the following amino acid sequences. Peptide 113-122=Ac-L V G R Q L E E F L-NH₂ (SEQ ID NO:498), Peptide 336-357=Ac-L L E Q L N E Q F N W V S R L A N L T Q G E-NH2 (SEQ ID NO:499), and Peptide 377-390=Ac-P S G V T E V V V K L F D S-NH₂ (SEQ ID NO:500).

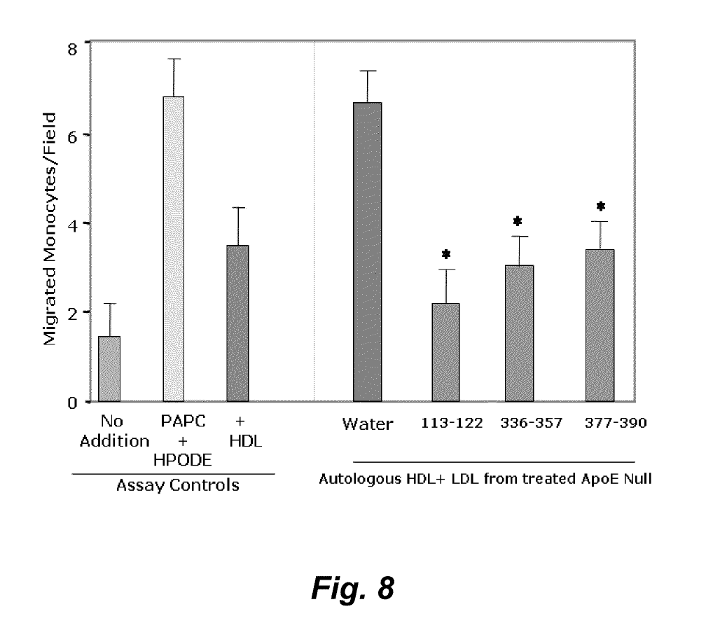

Each mouse received 200 μg of the peptide by stomach tube. Four hours later blood was obtained, plasma separated, lipoproteins fractionated and HDL (at 25 μg per ml) was assayed for protective capacity against the oxidation of LDL (at 100 μg per ml) in cultures of human artery wall cells. The data are shown in FIG. 8. The peptide afforded significant HDLprotective capacity in the mice.

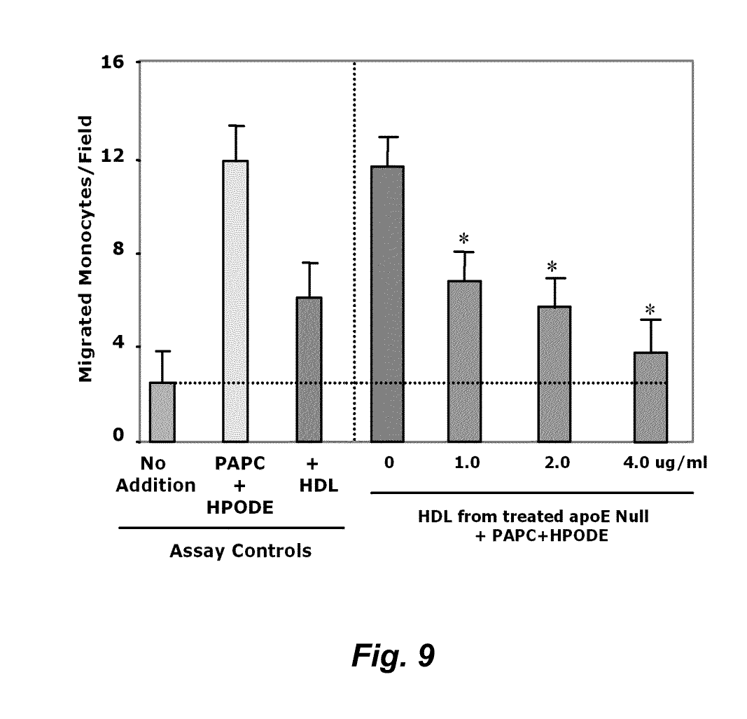

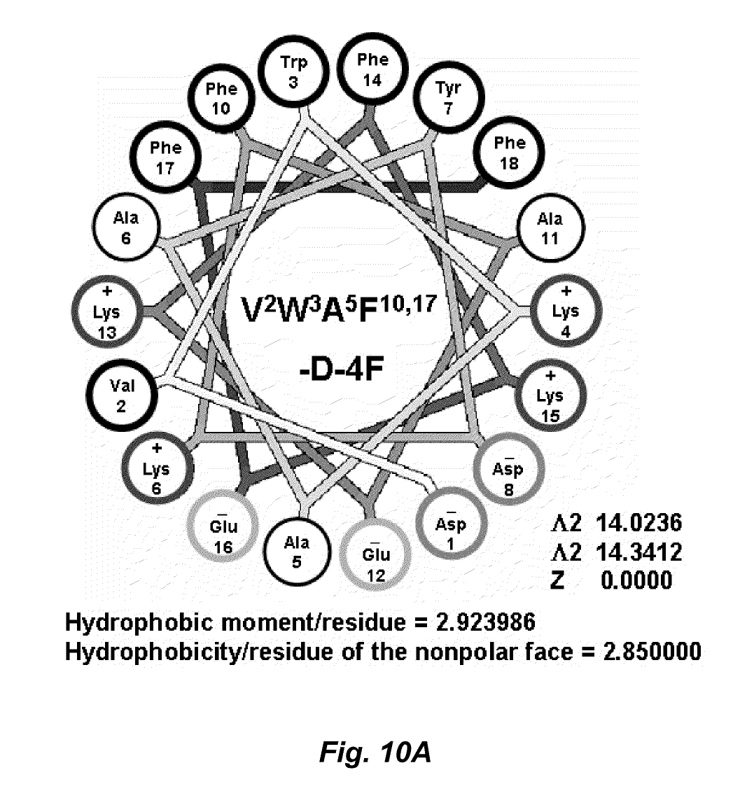

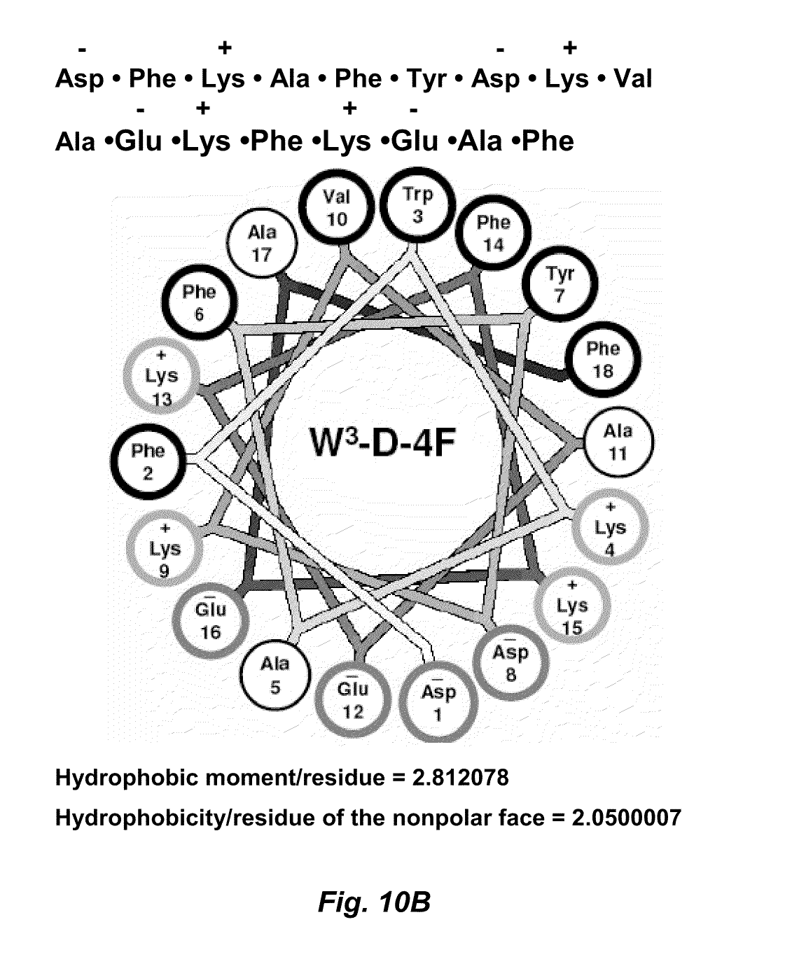

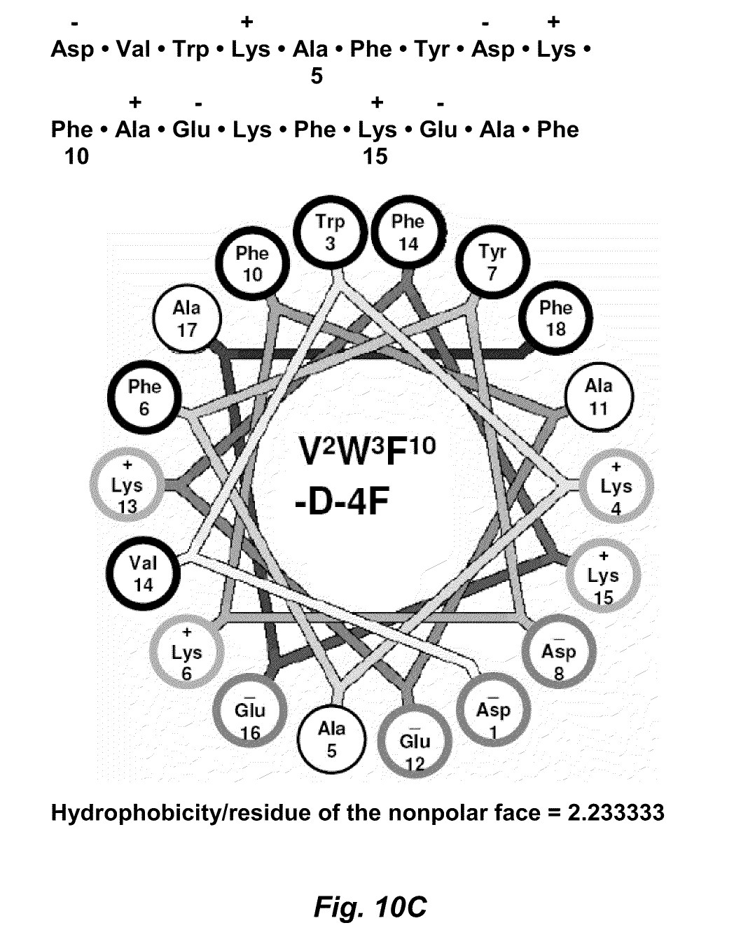

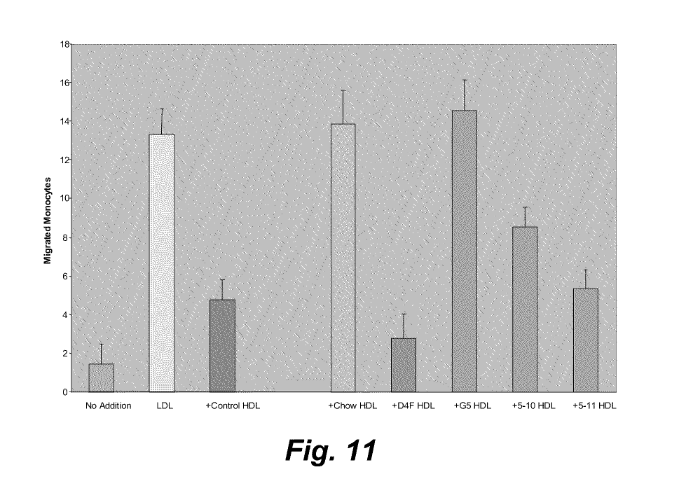

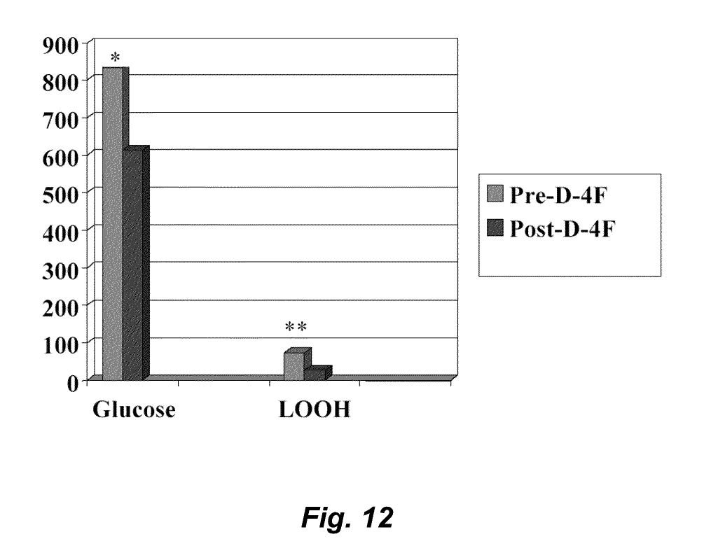

In another experiment, female, 4 month old apoE deficient mice (n=4 per group) were treated with the 11 amino acid G* peptide 146-156 with the sequence: Ac-Q Q T H M L D V M Q D-NH₂ (SEQ ID NO:501). The mice received the peptide in their drinking water at the indicated concentrations (see FIG. 9). Following eighteen hrs, blood was obtained, plasma separated, lipoproteins fractionated and HDL (at 50 μg cholesterol per ml) was assayed for protective capacity against the oxidation of PAPC (at 25 μg per ml)+HPODE (at 1.0 μg per ml) in cultures of human artery wall cells. Assay controls included No additions, PAPC+HPODE and PAPC+HPODE plus Control HDL (designated as +HDL). The data are mean+/−SD of the number of migrated monocytes in nine high power fields in triplicate cultures. Asterisks indicate significance at the level of p<0.05 vs. the water control (0 μg/ml).

### Example 3

### Solution Phase Chemistry for Peptide Synthesis

In certain embodiments, a solution-phase synthesis chemistry provides a more economical means of synthesizing peptides of this invention.

Prior to this invention synthesis was typically performed using an all-solid phase synthesis chemistry. The solid phase synthesis of peptides of less than 9 amino acids is much more economical than the solid phase synthesis of peptides of more than 9 amino acids. Synthesis of peptides of more than 9 amino acids results in a significant loss of material due to the physical dissociation of the elongating amino acid chain from the resin. The solid phase synthesis of peptides containing less than 9 amino acids is much more economical because the there is relatively little loss of the elongating chain from the resin.

In certain embodiments, the solution phase synthesis functions by converting the synthesis of the 18 amino acid apoA-I mimetic peptide, 4F (and other related peptides) from an all solid phase synthesis to either an all solution phase synthesis or to a combination of solid phase synthesis of three chains each containing, e.g., 6 amino acids followed by the assembly of the three chains in solution. This provides a much more economical overall synthesis. This procedure is readily modified where the peptides are not 18 amino acids in length. Thus, for example, a 15 mer can be synthesized by solid phase synthesis of three 5 mers followed by assembly of the three chains in solution. A 14 mer can be synthesized by the solid phase synthesis of two 5 mers and one 4 mer followed by assembly of these chains in solution, and so forth.

A Summary of Synthesis Protocol.

An illustrative scheme for the synthesis of the peptide D4F (Ac-D-W-F-K-A-F-Y-D-K-V-A-E-K-F-K-E-A-F-NH₂, (SEQ ID NO:13) is illustrated in Table 17. (The scheme and yields for the synthesis are shown in Table 17.

TABLE 17
Illustrative solution phase synthesis scheme.

| | | | | | | |
|---|---|---|---|---|---|---|
| | | | | Final | Wt. of Crude | Wt. of Pure |
| | | Fmoc | Coupling | Wt. of Resin | Peptide (gms) | Peptide (mg) |
| Synthesis | Resin | Amino Acid | Reagent | (gms) | Yield (%) | Yield ((%) |

Methods Used for D4F Synthesis

| | | | | | | |
|---|---|---|---|---|---|---|
| Stepwise | Rink Amide | 6 Equiv | HBTU/HOBT | 4 | 2.0 | 500 |
| Solid Phase | (1 mmole) | | | | 86 | 25 |
| | 1.8 gms | | | | | |
| Stepwise | Rink Amide | 2 Equiv | DIC/HOBT | 3.9 | 2.0 | 450 |
| Solid Phase | (1 mmole) | | | | 86 | 22.5 |
| | 1.8 gms | | | | | |
| Fragment | Rink Amide | | HBTU/HOBT | 3.3 | 1.0 | 100 |
| coupling | (1 mmole) | | | | 43 | 10 |
| (6 + 6 + 6) | 1.8 gms* | | | | | |

Synthesis of D4F Fragments

| | | | Fragment 1 (2HN-KFKEAF (SEQ ID NO: 502) on rink | | |
|---|---|---|---|---|---|
| | | | amide resin(K and E are properly protected) | | |
| Fragment 2 | Cl-TrT-Resin | 6 Equiv | HBTU/HOBT | 11 | 2.2 |
| 6 residues | (5 mmol) | | | | crude |
| stepwise | 6.5 gms | | | | protected |
| Solid Phase | | | | | |
| | | | | | 36 |
| | | | Fmoc-Y(But)-D(But)-K(Boc)-V-A-E(But)—COOH | | |
| | | | (SEQ ID NO: 503) | | |
| Fragment 2 | Cl-TrT-Resin | 6 Equiv | HBTU/HOBT | 10 | 1.8 |
| 6 residues | (5 mmol) | | | | crude |
| stepwise | 6.5 gms | | | | protected |
| Solid Phase | | | | | |
| | | | | | 32 |

Ac-D(But)-W-F-K(Boc)-A-F—COOH (SEQ ID NO: 504)

Synthesis by solution phase using fragments produced by the solid phase method.

| | |
|---|---|
| Fragment | Wang resin. C-terminal hexapeptide (subjected to ammonolysis). Yield quantitative. |
| 1. | NH2-K(Boc)-F-K(Boc)-E(But)-A-F-Wang resin (SEQ ID NO: 505) |
| | NH2-K(Boc)-F-K(Boc)-E(But)-A-F-CO—NH2 |
| | (SEQ ID NO: 506) |

Fragment 2 from above was coupled to
fragment 1 in DMF using DIC/HOBT.

Fmoc-Y(But)-D(But)-K(Bpc)-V-A-E(But)-K(Boc)-F-K(Boc)-E(But)-F-Co—NH2
(SEQ ID NO: 507) 12 residue peptide was characterized as free peptide after removing
protecting groups. Yield was 50%
| |

Fmoc from the above- 12 rtesidue was removed by piperidine in DMF (20%. After
drying the peptide was copied to Fragment 3 using DCl/HOBT in DMF.

Ac-D(But)-W-F-K(Boc)-A-F-Y(But)-D(but)-K(Boc)-V-A-E(But)-K(Boc)-F-K(Boc)-
E(But)-A-FCO-NH2 (SEQ ID NO: 508)
Protected peptide yield was quantitative.
Protecting groups removed using mixture of TFA (80%), phenol (5%), thioanisole (5%).
water)5%), triisopropylsilane (TIS, 5%), stirred for 90 min.
Precipitated by ether and purified by C-4 HPLC column. Yield 25%

B) Details of Synthesis Protocol.

1) Fragment Condensation Procedure to Synthesize D-4F

Fragments synthesized for fragment condensation on solid phase are:

Fragment 1:
(SEQ ID NO: 509)
Ac-D(OBut)-W-F-K(εBoc)-A-F-COOH;
Fragment 2:
(SEQ ID NO: 510)
Fmoc-Y(OBut)-D(OBut)-K(εBoc)-V-A-E(OBut)-COOH;
and
Fragment 3
(SEQ ID NO: 511)
Fmoc-K(εBoc)F-K(εBoc)-E(OBut)-A-F- Rink amide resin.

Fragment 1 was left on the resin to obtain final peptide amide after TFA treatment.

To synthesize fragment 1: Fmoc-Phe (1.2 equivalents) was added to chlorotrityl resin (Nova Biochem, 1.3 mMol/g substitution, 5 mMol or 6.5 g was used) in presence of six equivalents of DIEA in DMF: dichloromethane (1:1)) and stirred for 4 h. Excess of functionality on the resin was capped with methanol in presence of dichloromethane and DIEA. After the removal of Fmoc-Fmoc amino acid derivatives (2 equivalents) were added using HOBt/HBTU reagents as described above. Final Fmoc-D(OBut)-W-F-K(EBoc)-A-F Chlorotrityl resin was treated with Fmoc deblocking agent and acetylated with 6 equivalents of acetic anhydride in presence of diisoprolylethyl amine. The resulting Ac-D(OBut)-W-F-K(EBoc)-A-F-resin was treated with a mixture of triflouroethanol-acetic acid-dichloromethane (2:2:6, 10 ml/g of resin) for 4 h at room temperature. After removal of the resin by filtration, the solvent was removed by aziotropic distillation with n-hexane under vacuum. The residue (1.8 g) was determined by mass spectral analysis to be Ac-D(OBut)-W-F-K(εBoc)-A-F —COOH (SEQ ID NO:512).

Fragment 2, Fmoc-Y(OBut)-D(OBut)-K(EBoc)-V-A-E(OBut)-COOH (SEQ ID NO:513), was obtained using the procedure described for Fragment 1. Final yield was 2.2 g.

Fragment 3. 0.9 g (0.5 mmol) of Rink amide resin (Nova Biochem) was used to obtain fragment Rink amide resin was treated with 20% pipetidine in dichloromethane for 5 min once and 15 min the second time (Fmoc deblocking reagents).

1.2 equivalents of Fmoc-Phe was condensed using condensing agents HOBt/HBTU (2 equivalents in presence of few drops of diisopropylethyl amine) (amino acid condensation). Deblocking and condensation of the rest of the amino acids were continued to obtain the of Fmoc-K(EBoc)F-K(EBoc)-E(OBut)-A-F-rink amide resin (SEQ ID NO:514). Fmoc was cleaved and the peptide resin K(EBoc)F-K(EBoc)-E(OBut)-A-F-rink amide resin (SEQ ID NO:514) was used for fragment condensation as described below.

Fragment 2 in DMF was added to Fragment 3 (1.2 equivalents) using HOBt-HBTU procedure in presence of DIEA overnight. After washing the resin with DMF and deblocking Fmoc-Fragment 1 (1.2 equivalents) was added to the dodecapeptide resin using HOBt-HBTU procedure overnight.

The final peptide resin (3.3 g) was treated with a mixture of TFA-Phenol-triisopropylsilane-thioanisole-water (80:5:5:5) for 1.5 h (10 ml of the reagent/g of the resin). The resin was filtered off and the solution was diluted with 10 volumes of ether. Precipitated peptide was isolated by centrifugation and washed twice with ether. 1 g of the crude peptide was subjected to HPLC purification to obtain 100 mg of the peptide.

2) Characterization of Peptide.

The peptide was identified by mass spectral and analytical HPLC methods.

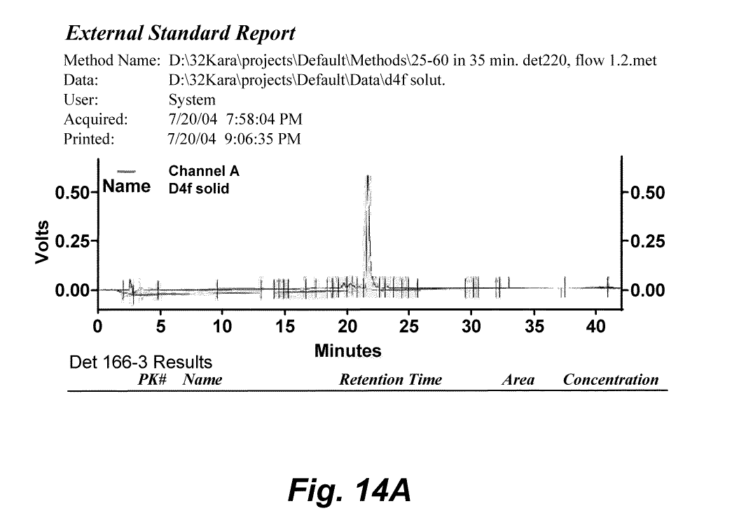

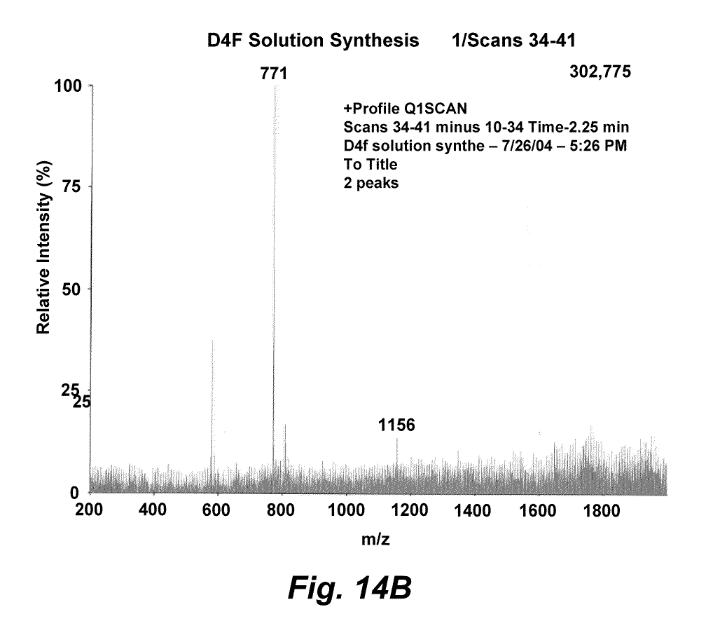

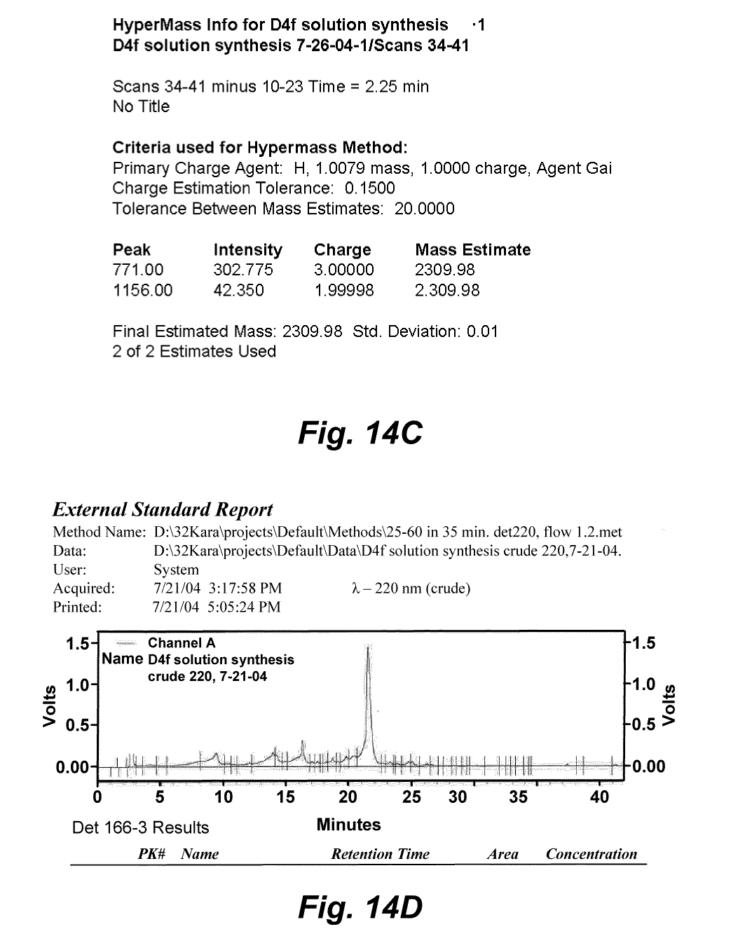

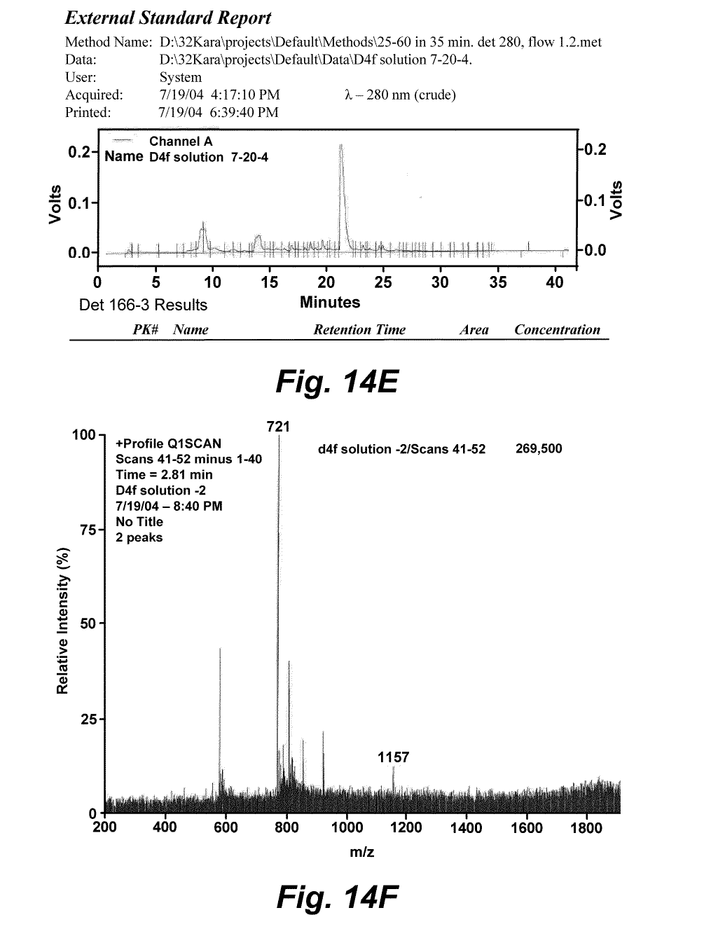

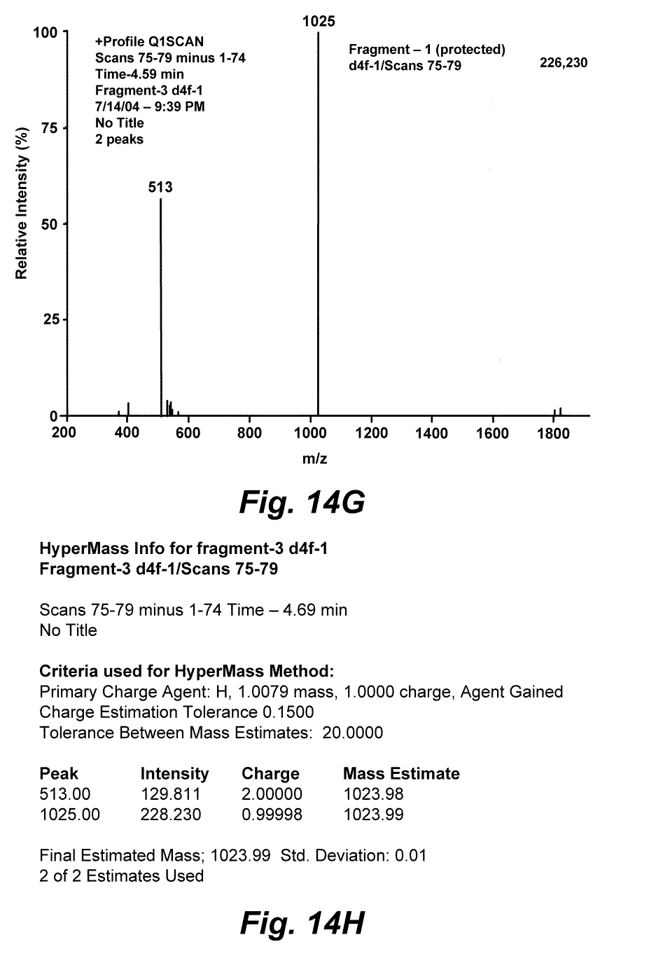

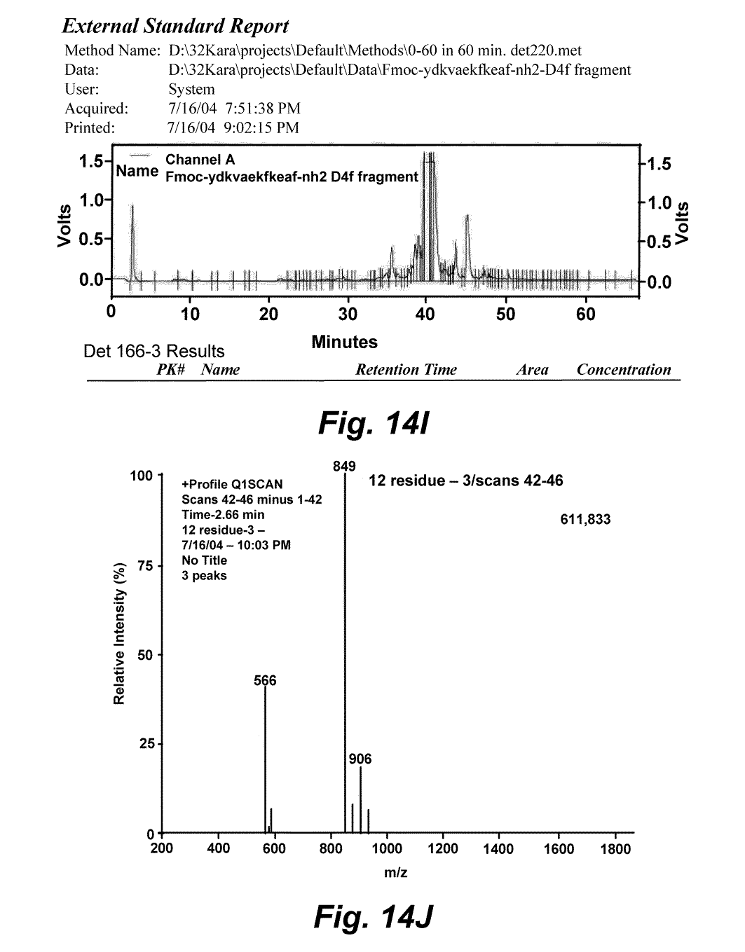

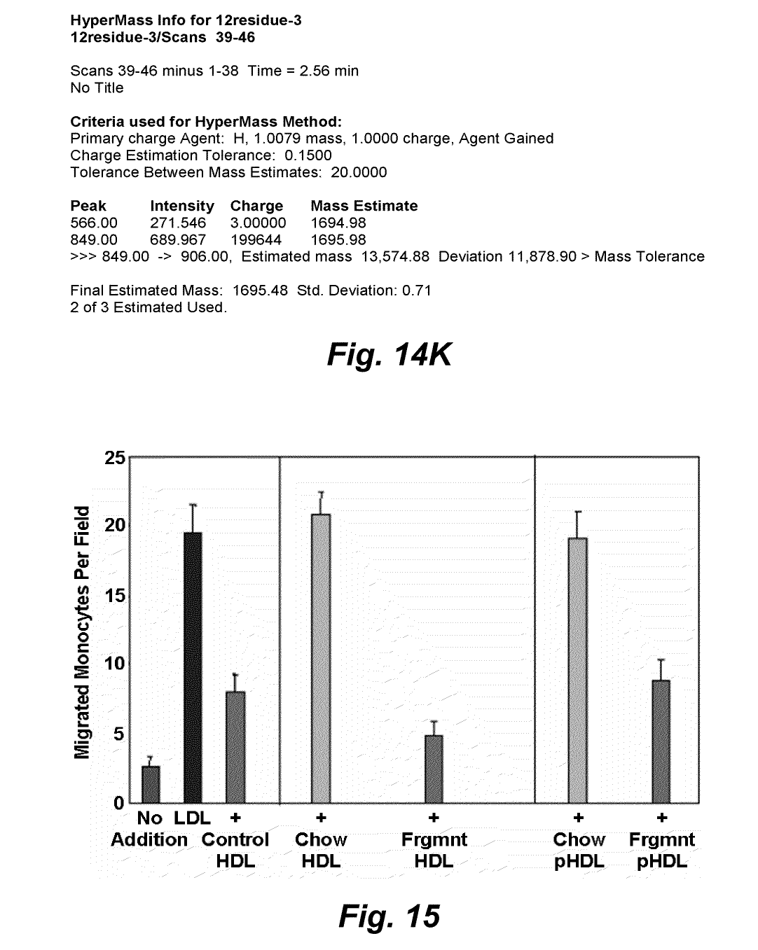

FIGS. 14A-14L demonstrate the purity of the resulting peptide. FIG. 15 demonstrates that the resulting peptide was biologically active in mice.

### Example 4

### G* Peptides Derived From Apo-M Increase Paroxynase Activity

Female apoE null mice 4 months of age (n=4 per group) were administered by intraperitoneal injection either scrambled D-4F (a non-active control peptide) or D-4F at 10 μg/mouse or the peptide Ac-KWIYHLTEGSTDLRTEG-NH₂ (SEQ ID NO:515) synthesized from L-amino acids (L-ApoM) at 50 μg/mouse. The mice were bled 2 or 6 hours later and their HDL isolated by FPLC and the paraoxonase activity in the HDL determined and plotted on the X-axis. Other 4-month-old female apoE null mice (n=4 per group) were administered by gastric gavage the peptide Ac-KWIYHLTEGSTDLRTEG-NH₂ (SEQ ID NO:515) synthesized from L-amino acids (L-ApoM) at 100 μg/mouse (L-ApoM by gavage). The mice were bled 6 hours later and their HDL isolated by FPLC and the paraoxonase activity in the HDL determined and plotted on the X-axis.

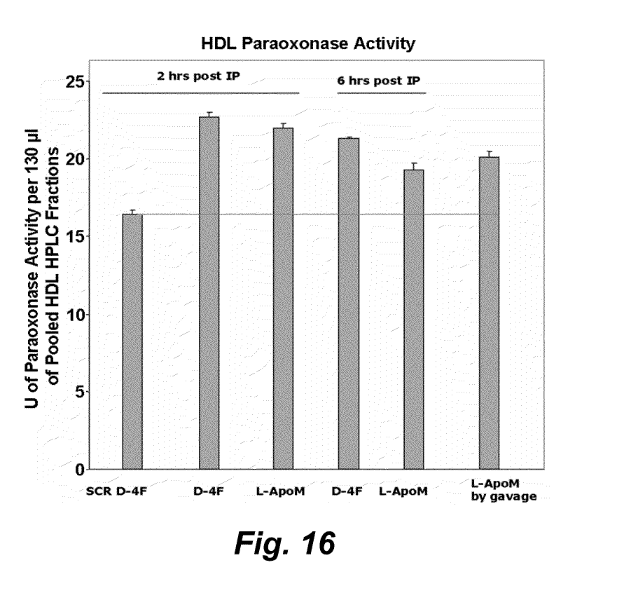

As shown in FIG. 16, administration of the sequence from apoM corresponding to residues 99-115 synthesized from L-amino acids and blocked at both the N and Carboxy terminals (SEQ ID NO: 515) and administered by intraperitoneal injection or gavage increased paraoxonase activity in apoE null mice.

### Example 5

### Activity of LAEYHAK (SEQ ID NO: 8) Peptide.

Five milligrams of the peptide LAEYHAK (SEQ ID NO: 8) synthesized from all D-amino acids was administered to each of four cynomologous monkeys in 2.0 mL of water by stomach tube and followed with 2.0 mL of water as a wash. Six hours later the monkeys were bled and their plasma fractionated by fast protein liquid chromatography (FPLC) and tested in human artery wall cell cultures.

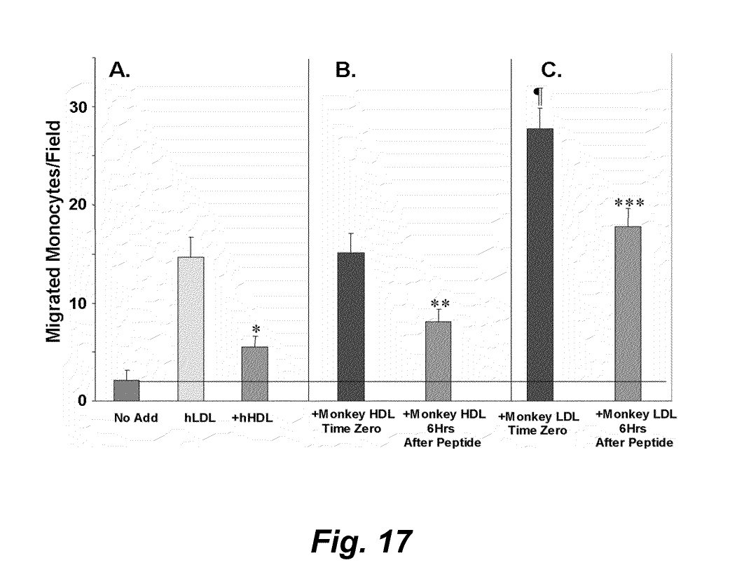

As shown in panel A of FIG. 17, addition to the cells of normal human LDL (hLDL) at a concentration of 100 μg/mL of LDL-cholesterol resulted in the production of monocyte chemotactic activity which is plotted on the y-axis of the Figure. Also as shown in panel A, addition to the cells of normal human HDL (hHDL) at a concentration of 50 μg/mL of HDL-cholesterol together with hLDL at a concentration of 100 μg/mL of LDL-cholesterol resulted in significantly less monocyte chemotactic activity.

As shown in panel B of FIG. 17, addition to the cells of hLDL at a concentration of 100 μg/mL of LDL-cholesterol together with monkey HDL at a concentration of 50 μg/mL of HDL-cholesterol taken at time zero (i.e. before administration of the peptide) did not reduce monocyte chemotactic activity. However, as also shown in panel B, addition of the monkey HDL at the same concentration but taken 6 hours after administration of the peptide significantly reduced monocyte chemotactic activity. As shown in panel C, addition to the cells of monkey LDL prior to the administration of peptide (Time Zero) at a concentration of 100 μg/mL of LDL-cholesterol resulted in significantly more monocyte chemotatic activity than addition of the same concentration of hLDL in panel A. As also shown in panel C, addition to the cells of the same concentration of monkey LDL taken 6 hours after administration of the peptide resulted in significantly less monocyte chemotactic activity.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

1. A peptide that ameliorates one or more symptoms of an inflammatory condition, wherein:
said peptide comprises the amino acid sequence LAEYHAK (SEQ ID NO: 8) or KAHYEAL (SEQ ID NO:516); and
said peptide comprises at least one D amino acid and/or at least one protecting group.

2. The peptide of claim 1, wherein said peptide comprises at least one D amino acid.

3. (canceled)

4. The peptide of claim 1, wherein said peptide comprises at least one protecting group.

5-8. (canceled)

9. A peptide that ameliorates one or more symptoms of an inflammatory condition, wherein said peptide:
ranges in length from about 3 to about 10 amino acids;
comprises an amino acid sequence wherein said sequence comprises acidic or basic amino acids alternating with one or two aromatic, hydrophobic, or uncharged polar amino acids;
comprises hydrophobic terminal amino acids or terminal amino acids bearing a hydrophobic protecting group;
is not the sequence LAEYHAK (SEQ ID NO: 8) comprising all L amino acids;
wherein said peptide converts pro-inflammatory HDL to anti-inflammatory HDL or makes anti-inflammatory HDL more anti-inflammatory.

10. A peptide that amelioriates one or more symptoms of an inflammatory condition, wherein said peptide comprises the amino acid sequence of a peptide found in Tables 3 or 14, or a concatamer thereof.

11. The peptide of claim 10, wherein said peptide comprises at least one D amino acid.

12. (canceled)

13. The peptide of claim 10, wherein said peptide comprises at least one protecting group.

14-17. (canceled)

18. A peptide that ameliorates one or more symptoms of an inflammatory condition, wherein:
said peptide comprises an amino acid sequence selected from the group consisting of DMT-Arg-Phe-Lys (SEQ ID NO:1), DMT-Arg-Glu-Leu (SEQ ID NO:2), Lys-Phe-Arg-DMT (SEQ ID NO:3), and Leu-Glu-Arg-DMT (SEQ ID NO:4), where DMT is dimethyltyrosine.

19-20. (canceled)

21. The peptide of claim 18, wherein said Arg is a D amino acid.

22-25. (canceled)

26. The peptide of claim 18, wherein said peptide is selected from the group consisting of BocDimethyltyrosine-D-Arg-Phe-Lys(OtBu) (SEQ ID NO:5), and BocDimethyltyrosine-Arg-Glu-Leu(OtBu) (SEQ ID NO:6).

27. (canceled)

28. A pharmaceutical formulation comprising the peptide of claim 10, and a pharmaceutically acceptable excipient.

29-30. (canceled)

31. The pharmaceutical formulation of claim 28, wherein the formulation is formulated for oral administration.

32. (canceled)

33. A method of ameliorating a symptom of atherosclerosis in a mammal, said method comprising administering to said mammal a peptide of claim 10 in an amount sufficient to ameliorate a symptom of atherosclerosis.

34-38. (canceled)

39. The method of claim 33, wherein said mammal is a mammal diagnosed as at risk for stroke or atherosclerosis.

40-41. (canceled)

42. A method of mitigating or preventing a coronary complication associated with an acute phase response to an inflammation in a mammal, wherein said coronary complication is a symptom of atherosclerosis, said method comprising administering to a mammal having said acute phase response, or at risk for said acute phase response, a peptide of claim 10.

43.-50. (canceled)

51. A method of ameliorating a symptom of diabetes in a mammal, said method comprising administering to said mammal one or more peptides of claim 10.

52-59. (canceled)

60. A method of inhibiting restenosis in a mammal, said method comprising administering to said mammal one or more peptides more active agents described in Tables 1-15 and/or a small organic molecule as described herein.

61-69. (canceled)

70. A stent for delivering drugs to a vessel in a body comprising: a stent framework including a plurality of reservoirs formed therein, and one or more active agents described in Tables 1-15 and/or a small organic molecule as described herein positioned in the reservoirs.

71-87. (canceled)

88. A method of manufacturing a drug-polymer stent, comprising: providing a stent framework; cutting a plurality of reservoirs in the stent framework; applying composition comprising one or more of the active agents described herein to at least one reservoir; and drying the composition.

89. (canceled)

90. A method of treating a vascular condition, comprising:
positioning a stent according to claim 70 within a vessel of a body;
expanding the stent; and
eluting at least one active agent from at least a surface of the stent.

91. A method of synthesizing a peptide, said method comprising:
providing at least 3 different peptide fragment subsequences of said peptide; and
coupling said peptide fragment subsequences in solution phase to form said peptide.

92-98. (canceled)
